(19) United States
(12) Patent Application Publication
(10) Pub. No.: US 2023/0116624 A1
(43) Pub. Date: Apr. 13, 2023

(54) METHODS AND SYSTEMS FOR ASSISTED FITNESS

(71) Applicant: CHENGDU FIT-FUTURE TECHNOLOGY CO., LTD., Sichuan (CN)

(72) Inventors: Tianguang TANG, Chengdu (CN); Qiang FU, Chengdu (CN); Lijun XUE, Chengdu (CN)

(73) Assignee: CHENGDU FIT-FUTURE TECHNOLOGY CO., LTD., Sichuan (CN)

(21) Appl. No.: 17/823,502

(22) Filed: Aug. 30, 2022

Related U.S. Application Data
(60) Provisional application No. 63/260,696, filed on Aug. 30, 2021.

(51) Int. Cl. A63B 24/00

(52) U.S. Cl. CPC A63B 24/0075; A63B 2024/0065; A63B 2214/00; A63B 2071/0638

(57) ABSTRACT

The embodiments of the present disclosure provide a apparatus and method for assisted fitness, the method may be performed based on the fitness apparatus. The method may include the following operations. At least one user using the fitness apparatus may be identified based on collected data, and an identification result may be determined, a control instruction may be obtained, and in response to the control instruction, and control content of the fitness apparatus corresponding to the control instruction may be executed based on the identification result.

### TECHNICAL FIELD

The present disclosure relates to fitness technology field, and in particular to a method for assisted fitness and a fitness apparatus.

### BACKGROUND

Along with the improvement of life standard, the acceleration of life rhythm has brought more pressure to people, thus people's pursuit of health has been paid more and more attention. There is no standardized standard for the current fitness management method. People usually choose to go to the gym to ask for a personal coach, or search for fitness guidance courses for learning. The price of personal coach in the gym is relatively high, and when people learn fitness guidance courses by themselves, there is a problem that they may not find suitable fitness guidance courses. At the same time, due to the lack of monitoring of fitness conditions, it is difficult for people to judge whether their fitness actions are standard in time and whether their own physical ability can support high-intensity training, which will lead to failure to achieve the target fitness effect or even damage to the body. Therefore, it is desirable to provide a method for assisted fitness and a fitness apparatus, which can help people to exercise scientifically and effectively with the help of suitable fitness apparatus.

### SUMMARY

One or more embodiments of the present disclosure provide a method for assisted fitness. The method may include the following operations. At least one user using the fitness apparatus may be identified based on collected data, and an identification result may be determined, a control instruction may be obtained, and in response to the control instruction, control content of the fitness apparatus corresponding to the control instruction may be executed based on the identification result.

One or more embodiments of the present disclosure provide a system for assisted fitness. The system may include an identification module configured to identify at least one user using the fitness apparatus based on collected data, and determine an identification result, a obtaining module configured to obtain a control instruction, and an execution module configured to execute control content of the fitness apparatus corresponding to the control instruction based on the identification result in response to the control instruction.

One or more embodiments of the present disclosure provide a fitness apparatus. The fitness apparatus may include a memory for storing instructions, at least one processor, the at least one processor in communication with the memory for executing the instructions, when executed by at least one processor, causing the fitness apparatus to perform operations. At least one user using the fitness apparatus may be identified based on collected data, and an identification result may be determined, a control instruction may be obtained, and in response to the control instruction, control content of the fitness apparatus corresponding to the control instruction may be executed based on the identification result.

One or more embodiments of the present disclosure provide a non-transitory computer-readable storage medium storing a set of instructions, when executed by at least one processor, causing the at least one processor to perform the method for assisted fitness.

### BRIEF DESCRIPTION OF THE DRAWINGS

The present disclosure will be further described by way of exemplary embodiments, which will be described in detail by means of the accompanying drawings. These examples are not limiting, and in these examples, the same numbers refer to the same structures, wherein:

FIG. 1 is a schematic diagram of an application scenario illustrating a system for assisted fitness according to some embodiments of the present disclosure;

FIG. 2 is a schematic diagram illustrating a user completing a control instruction through actions according to some embodiments of the present disclosure;

FIG. 3 is another schematic diagram illustrating a user completing a control instruction through actions according to some embodiments of the present disclosure;

FIG. 4 is a schematic diagram illustrating the system realizing the control content by recognizing user actions according to some embodiments of the present disclosure;

FIG. 5 is another schematic diagram illustrating the system realizing the control content by recognizing user actions according to some embodiments of the present disclosure;

FIG. 6 is an exemplary block diagram of a system for assisted fitness according to some embodiments of the present disclosure;

FIG. 7 is an exemplary flowchart of a method for assisted fitness according to some embodiments of the present disclosure;

FIG. 8 is a schematic diagram illustrating executing the control content of the fitness apparatus corresponding to the control instruction according to some embodiments of the present disclosure;

FIG. 9a is an exemplary flowchart of outputting interactive content corresponding to an interactive instruction according to some embodiments of the present disclosure;

FIG. 9b is a schematic diagram illustrating at least two users interacting using the same fitness apparatus according to some embodiments of the present disclosure;

FIG. 9c is a schematic diagram illustrating at least two users interacting using a plurality of fitness apparatus according to some embodiments of the present disclosure;

FIG. 10 is an exemplary flowchart of determining interactive content according to some embodiments of the present disclosure;

FIG. 11 is an exemplary flowchart of determining interactive content according to other embodiments of the present disclosure;

FIG. 12 is a schematic diagram of arranging a plurality of external cameras (or camera modules) in a fitness place according to some embodiments of the present disclosure;

FIG. 13 is another exemplary flowchart of arranging a plurality of external cameras (or camera modules) in a fitness place according to some embodiments of the present disclosure;

FIG. 14 is a schematic diagram illustrating an external camera (or a camera module) having a plurality of freedom degrees in space according to some embodiments of the present disclosure;

FIG. 15 is a schematic diagram illustrating the external camera (or a camera module) adjusting its own shooting angle based on the captured human body image and tracking the portrait within the field of view according to some embodiments of the present disclosure;

FIG. 16 is another schematic diagram illustrating the external camera (or a camera module) adjusting its own shooting angle based on the captured human body image and tracking the portrait within the field of view according to some embodiments of the present disclosure;

FIG. 17 is another schematic diagram illustrating the external camera (or a camera module) adjusting its own shooting angle based on the captured human body image and tracking the portrait within the field of view according to some embodiments of the present disclosure;

FIG. 18 is a schematic diagram of generating fitness guidance courses using the course generation model according to some embodiments of the present disclosure;

FIG. 19 is an exemplary flowchart illustrating a fusion display method based on the user's human skeleton information and the coach's human skeleton information according to some embodiments of the present disclosure;

FIG. 20 is an exemplary block diagram of an apparatus for assisted fitness according to some embodiments of the present disclosure.

### DETAILED DESCRIPTION

In order to illustrate technical solutions of the embodiments of the present disclosure more clearly, the following briefly illustrates drawings in the illustration of the embodiments. Drawings in the following illustration are merely some examples or embodiments of the present disclosure. For those skilled in the art, the present disclosure may be applied to other similar scenarios in accordance with the drawings without creative works. Unless obviously obtained from the context or the context illustrates otherwise, the same number in the drawings refers to the same structure or operation.

It should be understood that “system”, “apparatus”, “unit”, and/or “module” used herein are a method for distinguishing different components, elements, members, parts, or assemblies of different levels. However, if other words may achieve the same purpose, the words may be replaced by other expressions.

As used in the disclosure and the appended claims, the singular forms “a,” “an,” and “the” include plural referents unless the content clearly dictates otherwise. In general, the terms “comprising” and “including” only prompt steps and elements that are explicitly identified, and these steps and elements do not constitute an exclusive list. Methods or apparatus may also include other steps or elements.

Flowcharts are used in the present disclosure to illustrate the operations performed by the system according to some embodiments of the present disclosure. It should be understood that the front or rear operations may not be necessarily performed exactly in order. On the contrary, each step may be performed in reverse or simultaneously. At the same time, other operations may also be added to the procedures, or a certain step or several steps may be removed from the procedures.

A system for assisted fitness may include one or more fitness apparatus. A fitness apparatus may assist a user in fitness, and during the fitness process, the apparatus may have various interactions with the user. More descriptions of the users who use fitness apparatus, reference may be found in the related descriptions in the user section below.

In some embodiments, when the user performs fitness based on the system for assisted fitness, the system for assisted fitness may obtain various data, and affect the related process of the user's fitness based on the obtained data. More descriptions of the obtained data, reference may be found in the related descriptions in the obtained information section below.

In some embodiments, the affecting the related process of the user's fitness may include follow operation. When the user performs fitness based on the system for assisted fitness, the system for assisted fitness may adjust or correct the related process of fitness in real time. More descriptions of adjustments or corrections of the related processes of fitness, reference may be found in the related descriptions in the corrective information section below.

In some embodiments, the related process of fitness may include follow operation. When the user performs fitness based on the system for assisted fitness, fitness may be performed based on a fitness guidance course. In some embodiments, fitness guidance course may also be referred to as fitness course. More descriptions of the fitness guidance course, reference may be found in the related descriptions in the fitness guidance course section.

In some embodiments, when the user performs fitness based on the system for assisted fitness, the system for assisted fitness may obtain the feedback information input by the user and give a response. For example, a user inputs control instructions, and the system for assisted fitness may respond based on the received control instructions. It should be noted that this is not limited to systems in the field of fitness, but includes any system that may respond based on user feedback. More descriptions of the user feedback, reference may be found in the related descriptions in the user feedback section below.

In some embodiments, when the user performs fitness based on the system for assisted fitness, the system for assisted fitness may further include various assisted functions. More descriptions of the assisted functions, reference may be found in the related descriptions of other functions below.

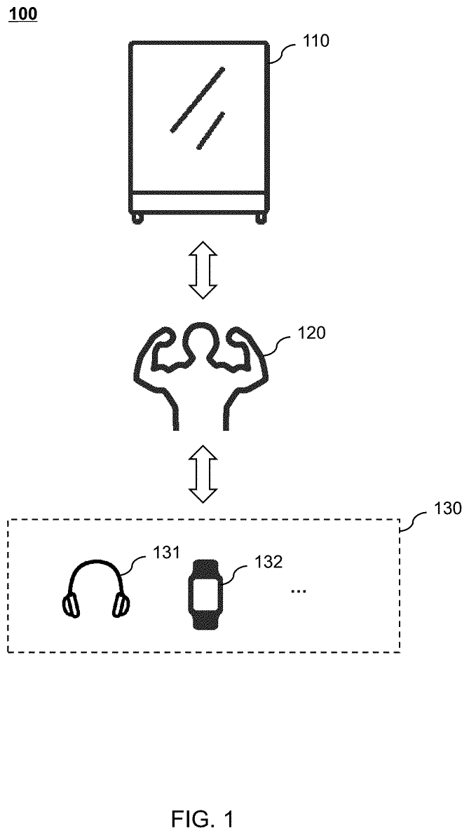

FIG. 1 is a schematic diagram of an application scenario illustrating a system for assisted fitness according to some embodiments of the present disclosure. In some embodiments, the application scenario 100 of the system for assisted fitness may include a fitness apparatus 110, a user 120 and a user terminal 130.

The fitness apparatus 110 may refer to an apparatus for assisting a user to perform fitness training. The fitness apparatus 110 may be of various types, such as a smart fitness mirror, or the like. In some embodiments, the fitness apparatus 110 may consist of one or more of a display apparatus, a processing apparatus, a voice apparatus, or the like. The display apparatus may include a display and/or mirror glass. For example, the display apparatus may be consisted of at least one display and at least one mirror glass, the mirror glass may reflect and transmit light in a certain ratio, and the mirror glass may enable the user 120 to not only see its own reflection image on the mirror, but also to see fitness guidance courses on the monitor. In some embodiments, fitness apparatus 110 may also be referred to as an apparatus for assisted fitness.

The fitness apparatus 110 may communicate information/data with the user 120. In some embodiments, while displaying preset content for the user 120, the display apparatus may also display real-time fitness information of the user 120 using the apparatus. For example, the display apparatus of the fitness apparatus 110 may display a preset fitness guidance course (e.g., video or image, etc.) for the user 120, and display fitness data when the user 120 performs fitness according to the fitness guidance course. The fitness data may include images and videos of the fitness actions of the user 120, physiological data of the user 120 during fitness, or the like. In some embodiments, while playing the preset content for the user 120, the voice apparatus may also play real-time fitness information of the user 120 using the apparatus. For example, the voice apparatus of the fitness apparatus 110 may play a preset fitness guidance course (e.g., voice guidance, etc.), and play fitness data when the user 120 performs fitness according to the fitness guidance course. In some embodiments, the fitness data may also include audio made by the user 120 while performing fitness. More descriptions of obtaining the fitness data of the user, reference may be found in the related descriptions of the user behavior information below. In some embodiments, the user 120 may interact with the fitness apparatus 110 through gesture operations, touch screen clicks, infrared remote control, or the like. For example, the user 120 may interactively select a fitness guidance course. In some embodiments, the fitness apparatus 110 may also include various other apparatus, including but not limited to positioning apparatus, image processing apparatus, or the like.

The user 120 may refer to a group of people who perform fitness training through the fitness apparatus 110. The same fitness apparatus 110 may correspond to one or more users 120. The users 120 may include various groups of people with different ages, genders, and physical qualities. In some embodiments, the fitness apparatus 110 may personalized display a fitness guidance course according to one or more users 120. For example, the fitness apparatus 110 may display interactive content according to the interactive actions of the plurality of users 120. In some embodiments, the user 120 may choose a fitness guidance course according to the requirements.

The user terminal 130 may refer to a terminal apparatus used by a user. In some embodiments, the user terminal 130 may include one or any combination of an earphone 130-1, a sports bracelet 130-2, and other various wearable apparatus. The user terminal 130 may be used to input and receive information and/or data related to the application scenario 100. For example, the user terminal 130 may detect the basic physiological information of the user 120 or the like, and transmit the basic physiological information to the fitness apparatus 110. For another example, the user terminal 130 may receive such as reminder information of a fitness action sent by the fitness apparatus 110.

In some embodiments, a method for fitness based on a system for assisted fitness may include: a system for assisted fitness (e.g., a fitness apparatus or a server) obtaining a fitness guidance course and displaying the fitness guidance course on a display apparatus of the fitness apparatus.

Triggering the obtaining of a fitness guidance course may be in a variety of ways. In some embodiments, the system for assisted fitness may obtain the fitness guidance course based on the course request instruction input by the user on the fitness apparatus. In some embodiments, the system for assisted fitness may automatically trigger the obtaining of a fitness guidance course. For example, the system for assisted fitness may recognize the fitness intention of a user and automatically trigger the obtaining of a fitness guidance course. The fitness intention may be the willingness to fitness directly or indirectly expressed through various behaviors of the user. The fitness intent may be determined by one or more of the user's appearances, the user action, the user's location, the user's expression, or the like. For example, if the infrared sensing apparatus of the fitness apparatus senses that the distance from the user to the fitness apparatus is less than a preset threshold, it may be determined that the user has fitness intentions. For another example, the system for assisted fitness may determine that the user has fitness intentions based on the appearance image of the user (e.g., the user is wearing sportswear, etc.) captured by the camera. In another example, the system for assisted fitness may also determine the fitness intention in other ways, for example, it may be determined that the user has the fitness intention through speech recognition of a dialogue related to fitness, and the method for determining the fitness intention is not limited herein.

The fitness guidance course may be in various forms, for example, the fitness guidance course may include one or more combinations of video, image, voice, text, or the like. The fitness guidance course may be derived from one or more of pre-recording, real-time generation, real-time adjustment based on pre-recording, or the like. For example, a pre-recorded fitness guidance course may be stored in a storage apparatus, and when the obtaining of the fitness guidance course is triggered, the system for assisted fitness directly obtains the fitness guidance course from the storage apparatus. The storage apparatus may be located in the fitness apparatus or in an external apparatus other than the fitness apparatus. More descriptions of the fitness guidance course, reference may be found in the related descriptions in the fitness guidance courses section below.

In some embodiments, the method for fitness based on the system for assisted fitness may include: the system for assisted fitness obtaining the fitness data of the user.

The fitness data may be the data generated by the user using fitness apparatus to fitness. For example, the fitness data may be data generated by the user performing fitness based on the guidance of the fitness guidance course.

The fitness data may be obtained in various ways. For example, a camera of a fitness apparatus may capture images or videos of user actions during fitness process. For example, the camera may be a depth-of-field lens.

In some embodiments, the method for fitness based on the system for assisted fitness may include: the system for assisted fitness determining the guidance information of the fitness process and displaying the guidance information on the display apparatus of the fitness apparatus based on the fitness data.

The guidance information of the fitness process may be a suggestion or reminder for the user in the fitness process, and may be used to guide the user to fitness. In some embodiments, the system for assisted fitness may guide the user's location. For example, the system for assisted fitness may determine whether the user is within the preset position range according to the image data of the user obtained by the camera. In response to a determination that the user is not within the preset position range, the system for assisted fitness may send reminder information to guide the user to adjust the position to be more convenient for shooting. In response to a determination that the user is within the preset position range, the system for assisted fitness may do not prompt or encourage likes. The camera may be a camera on the fitness apparatus, or may be other cameras connected to the fitness apparatus and located outside the fitness apparatus, for example, other cameras located in the fitness place.

The preset position range may be customized according to the situation. For example, the preset position range may be the position range where the camera may capture the entire body of the user.

The reminder information may include: information to remind the user that the position is deviated and/or to remind the user to move, etc., for example, reminding the user to move toward the middle of the camera's field of view.

In some embodiments, the system for assisted fitness may guide the user actions. For example, the system for assisted fitness may judge whether the user action is standard. If it is not standard, the system for assisted fitness may send reminder information to guide the user's fitness action to be more standard. If it is standard, the system for assisted fitness may not prompt or encourage likes, and may start counting the user actions and assessing the exercise consumption.

In some embodiments, the system for assisted fitness judging whether the action done by the user is standard may include: judging whether the action done by the user matches the action specified in the current course, judging whether the user action reaches a specified range, etc. For example, by setting the position of the standard action on the fitness apparatus, the system for assisted fitness may determine whether the user action is standard according to the distance between the position of the user action and the position of the standard action. For another example, by identifying key points (e.g., wrists, elbows, shoulders, etc.) in images of the user when performing fitness, the system for assisted fitness may determine whether the user action is standard according to the difference between the position of the key points and the standard position or the difference between the contour formed by the key points and standard contour the user action. More descriptions of standard actions, reference may be found in the related descriptions in the corrective information section.

The reminder information may include: the reminder information of the irregular fitness action, and/or the information of prompting the fitness action being adjusted, etc.

] The reminder mode may include, but is not limited to, one or more of images, texts, sounds, vibration, or other modes. In some embodiments, reminders may be issued in various ways. For example, the reminder of images and texts may be issued by the display apparatus of the fitness apparatus, or may be issued by the terminal apparatus carried by the user. As another example, the reminder of sounds may be issued by the voice apparatus of the fitness apparatus. For another example, the reminder of vibration may be issued by a wearable apparatus, and the wearable apparatus and the fitness apparatus may be connected by a wired network or a wireless network.

In some embodiments, the system for assisted fitness may also guide the content other than the user actions during the fitness process. For example, the user may be guided on whether they need to supplement energy, hydration, and/or whether they need to change their clothing. For example, after the user finishes a certain fitness guidance course or all fitness guidance courses of the day, the system for assisted fitness may prompt the user to supply water. For another example, the system for assisted fitness may also issue an energy supplement prompt to the user according to the user's fitness consumption and in combination with the user's personalized information (e.g., the user's basal metabolism, training requirements set by the user). For another example, the system for assisted fitness may prompt the user to change clothes according to the switching of the fitness guidance course or the change of the environmental information.

When using any system (not limited to a system for assisted fitness), a user may input feedback information (e.g., a control instruction), and any system may respond based on the feedback information (e.g., control instructions).

The control instruction may be set in various ways. In some embodiments, the control instruction may be preset by any system. In some embodiments, the user may select a certain control content in any system setting list, and customize its corresponding preset action, the preset action may be executed as a control instruction, and the execution result is the realization of the control content. For example, the user may set the instruction to increase the volume in the system setting list as: pointing up and holding for 2 seconds. In some embodiments, the user may select one or more control contents in the system setting list as the common control contents according to personal preference. In some embodiments, when the user triggers instruction recognition, the common control content may be preferentially displayed on the display apparatus of the system. For example, the user may select “shut down” and “increase the volume” as the common control contents. When the user triggers the system for assisted fitness to perform instruction recognition, the two items of “shut down” and “increase the volume” may be preferentially displayed on the display apparatus of the fitness apparatus. Selecting the common control content to be displayed on the display apparatus of the system first may provide users with convenient operations, facilitate users to quickly select control content according to their own habits, and improve user experience.

The triggering of control instruction recognition may be in various ways. In some embodiments, the system for assisted fitness may trigger the instruction recognition based on the user's operation of triggering the instruction recognition button on the fitness apparatus. In some embodiments, the system for assisted fitness may automatically trigger instruction recognition. For example, when the system for assisted fitness recognizes that the user action is similar to the preset action of the control instruction, the instruction recognition may be automatically triggered. In some embodiments, the system for assisted fitness may also trigger instruction recognition in other ways. For example, the system for assisted fitness may obtain the user's voice instruction to trigger instruction recognition, or the like.

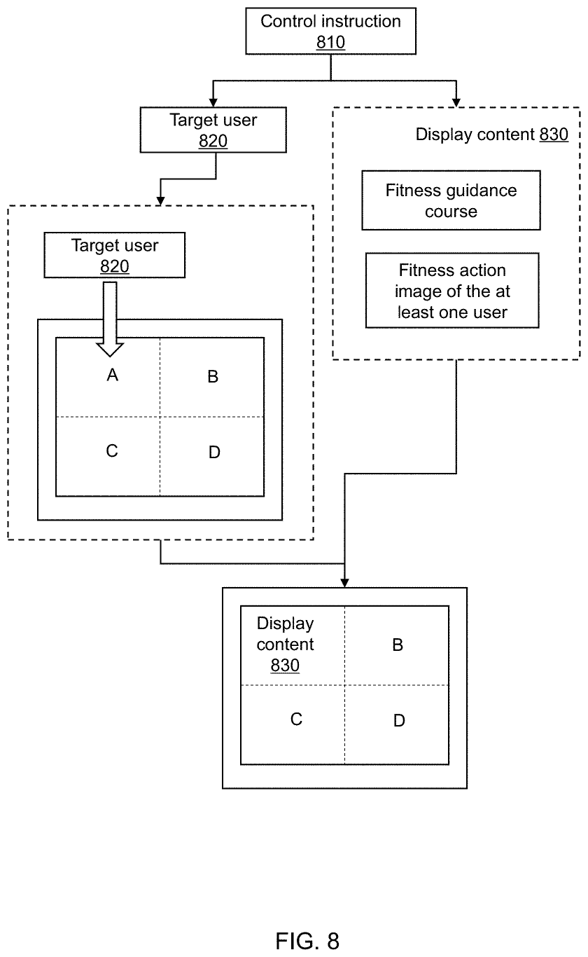

The user may issue the control instruction in various ways based on the preset action corresponding to the control instruction. In some embodiments, the preset action corresponding to the control instruction may be displayed to the user, so that the user may issue the control instruction based on the displayed content. For example, the display apparatus of any system may be divided into one or more areas or have one or more icons to display preset actions and their corresponding control contents. The user may select the desired control content according to the displayed content, and execute a preset action corresponding to the control content. More descriptions of the displayed content, reference may be found in the related contents of FIG. 8.

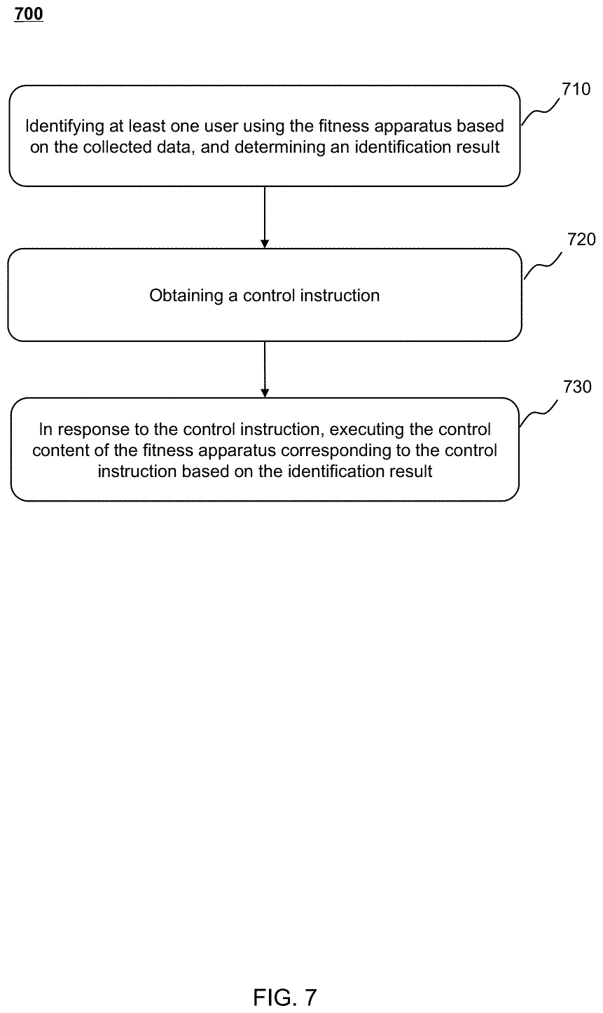

In some embodiments, the camera may capture real-time image data of the user, that is, capture images or videos of the user issuing a control instruction. Image data may be represented in various forms, such as images or videos. More descriptions of the image data, reference may be found in the related contents of FIG. 7. In some embodiments, the system may identify user actions in the image data and match their corresponding control instructions. In some embodiments, the system may identify the image in various ways to determine the control instruction issued by the user. For example, the system may identify the key points of the user in the image (e.g., joints of the hand, etc.), determine the type of the user's posture (e.g., determining the user action), and determine the control instruction issued by the user. For another example, the system may determine the control instruction issued by the user according to the matching degree between the standard image corresponding to the control instruction and the user image. The standard image may include a preset action image stored by the system corresponding to the control instruction. For example, in the system for assisted fitness, the standard image corresponding to the control instruction of increase the volume may be a finger pointing up. The standard image may be derived from the self-action image input by the user when presetting, the action model image constructed by the system, or the general image downloaded from the network. In some embodiments, the system may also match the corresponding control instruction in other ways based on the image data. For example, the image data feature in the image data may be determined by the model, and the corresponding control instruction may be determined according to the image data feature.

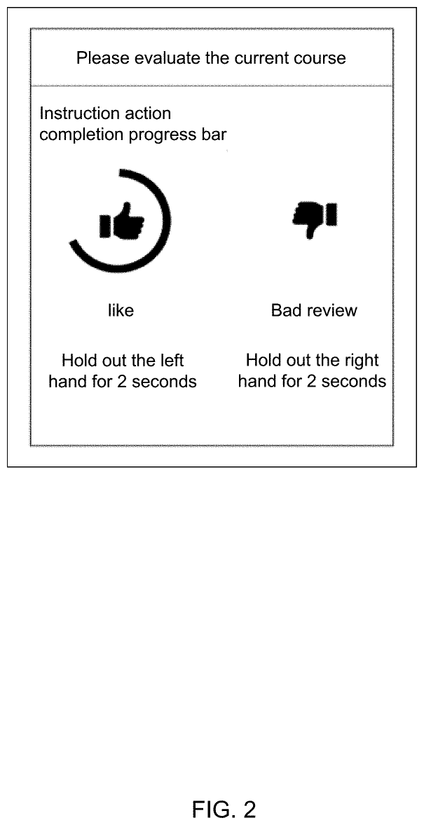

In some embodiments, the system may remind the user whether the preset action corresponding to the control instruction is completed in various ways. The reminder method is not limited to one or more of images, texts, sounds, and vibrations. For example, as shown in FIG. 2, to perform a like operation, the user needs to extend left hand for 2 seconds, and a completion progress bar may be displayed at the position corresponding to “like” on the screen to prompt the user whether the action is completed.

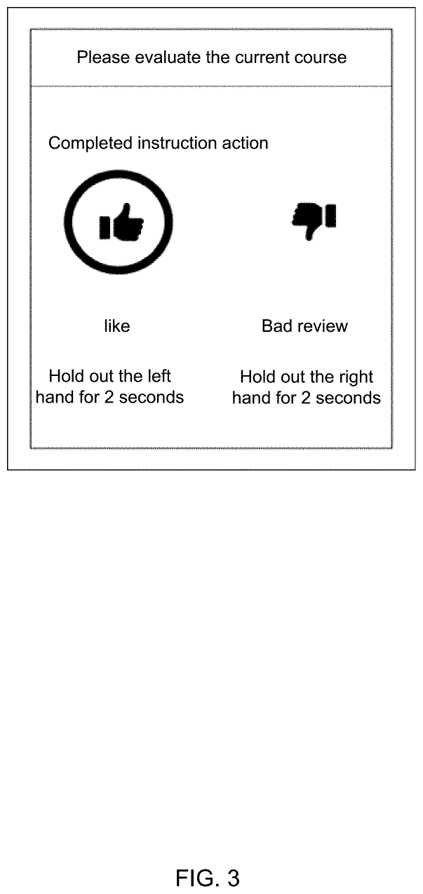

In some embodiments, when the user completes the preset action corresponding to the control instruction, the execution of the control instruction may be triggered. As shown in FIG. 3, when the user extends left hand for 2 seconds and the action is completed, the control instruction corresponding to “like” may be issued.

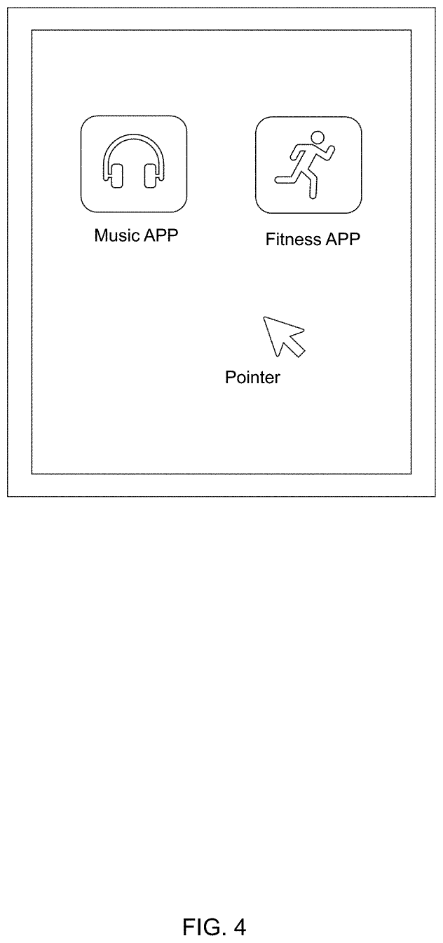

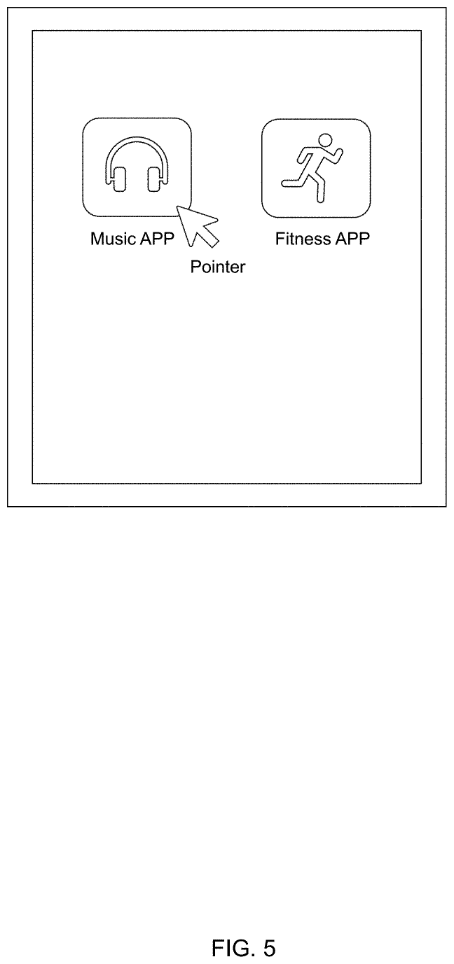

In some embodiments, at least one area or icon may be displayed on the display apparatus of the system, and at least one area or icon represents corresponding at least one control content. At the same time, at least one pointer may also be displayed on the display apparatus of the system. The system may control the pointer by identifying actions of the user's specific key points, for example, controlling the movement of the pointer, selecting an icon by the pointer to realize the control content corresponding to the icon, etc., determine the control instruction issued by the user through the interaction between the pointer and the area or icon. As shown in FIG. 4 and FIG. 5, the icons of a plurality of APPs may be displayed on the screen, and the system may control the pointer by recognizing the actions of the user's wrist. For example, when the wrist moves up, the pointer may also move up, and when the pointer touches an APP, the system may open the APP. When the pointer overlaps with the icon of an APP, and the user may maintain the pointer to overlap the APP icon for more than 2 seconds, the system may open the APP. When the pointer coincides with the icon of an APP, the user may complete a specified action, such as punching, to confirm that the pointer selects the APP.

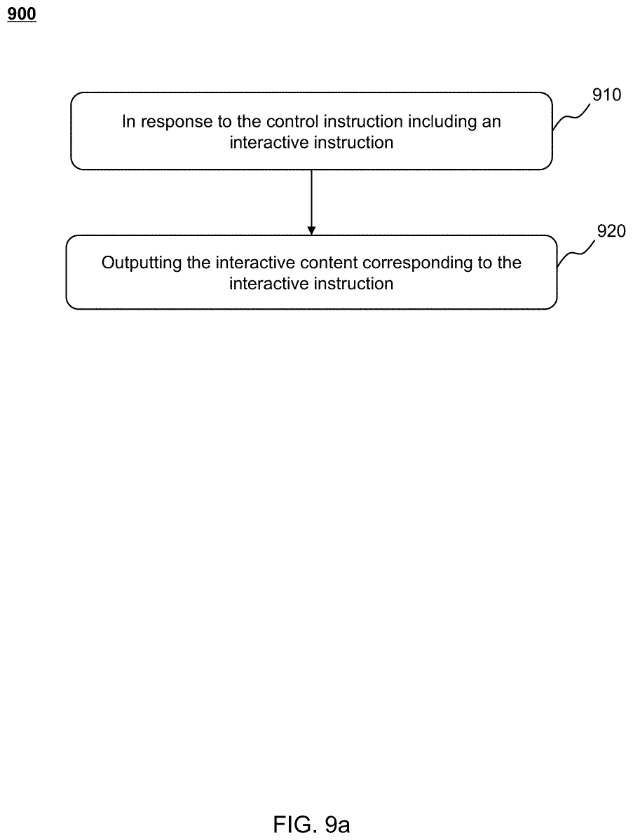

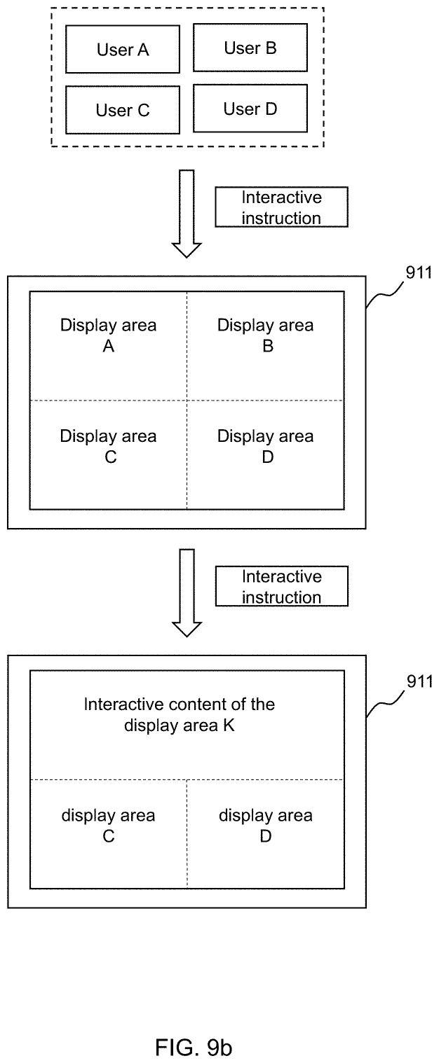

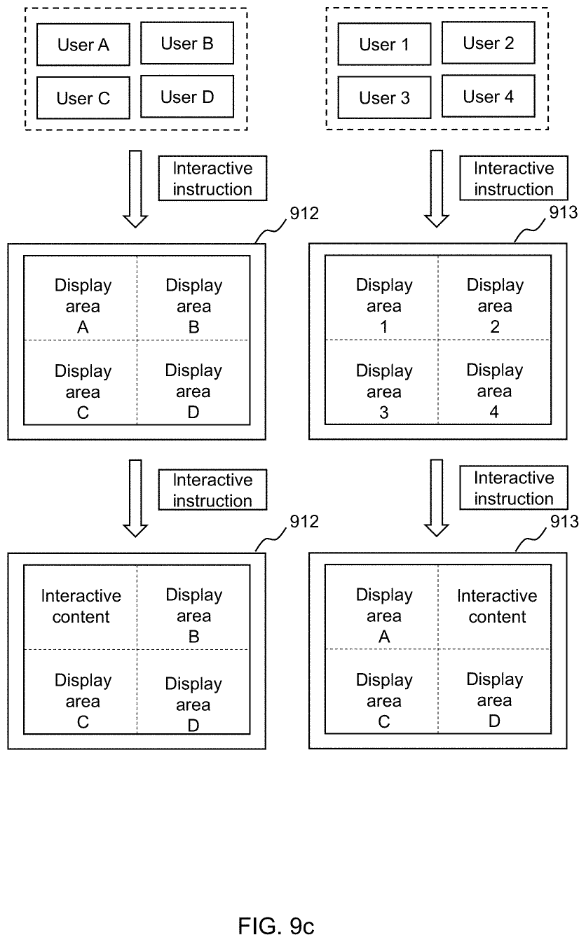

It may be understood that the controlling specific key points of the pointer may be set by user customization. For example, the specific key points may include the user's wrist joint or the user's eyeball, or the like. When the pointer coincides with the icon or area, the specified action that triggers the control content corresponding to the icon or area may also be customized. More descriptions of the control instruction and control content, reference may be found in FIG. 7, FIG. 8, FIG. 9, and their related descriptions.

In some embodiments, the system may determine the user actions through data of external IMU sensors. For example, external IMU sensors may be provided on the user's wearable apparatus or fitness apparatus. In some embodiments, the system may also determine the user actions in other ways. For example, the user action may be determined by image recognition performed by an image processing apparatus.

More descriptions of the user's feedback, reference may be found in the related descriptions of the user feedback section below.

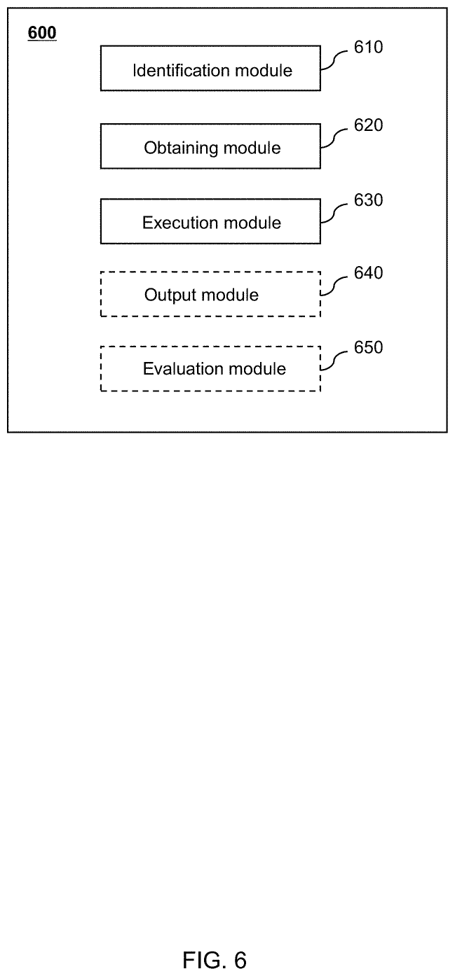

FIG. 6 is an exemplary block diagram of a system for assisted fitness according to some embodiments of the present disclosure. As shown in FIG. 6, the system for assisted fitness 600 may include an identification module 610, an obtaining module 620 and an execution module 630.

The identification module 610 may be configured to identify at least one user using the fitness apparatus based on the collected data, and determine an identification result.

More descriptions of the definition and description of the collected data and the identification result, as well as the method for determining the identification result, reference may be found in FIG. 7 and its related descriptions.

The obtaining module 620 may be configured to obtain a control instruction.

More descriptions of the definition and descriptions of the control instruction, and the method for obtaining the control instruction, reference may be found in FIG. 7 and its related descriptions.

The execution module 630 may be configured to in response to the control instruction, execute the control content of the fitness apparatus corresponding to the control instruction based on the identification result.

In some embodiments, the execution module 630 may also be configured to determine a target user based on the identification result, the target user being the user who issues the control instruction among the at least one user, and determine the control content based on the function icon pointed by a target pointer, the target pointer being a pointer corresponding to the target user.

In some embodiments, the execution module 630 may also be configured to determine a target user based on the control instruction, the target user being a user who issues the control instruction among the at least one user, determine the control content corresponding to the target user based on the target user and the control instruction, and execute the control content.

In some embodiments, the execution module 630 may also be configured to determine a target user based on the control instruction, the target user being a user who issues the control instruction among the at least one user, determine the display content based on the control instruction, determining a display area of the target user in the display apparatus of the fitness apparatus based on the target user, and display the display content on the display apparatus based on the display area.

More descriptions of the definition and descriptions of the control content, and the method for executing the control content of the fitness apparatus corresponding to the control instruction, reference may be found in FIG. 7, FIG. 8, and their related descriptions.

In some embodiments, the system for assisted fitness 600 may also include an output module 640. In response to the control instruction including an interactive instruction, and at least two users corresponding to the interactive instruction using a same fitness apparatus, the output module 640 may be configured to output interactive content corresponding to the interactive instruction, and the interactive content includes interactive fitness data.

In some embodiments, in response to the control instruction including an interactive instruction, the output module 640 may further be configured to obtain interactive reference information, the interactive reference information including at least one of an interactive mode between interactive users, a number of the interactive users, and the identification result of the interactive users, the interactive users being at least two users participating in interaction, determine interactive content corresponding to the interactive instruction based on the interactive reference information, and output the interactive content.

More descriptions of the definition and descriptions of the interactive instruction, the interactive content and the interactive reference information, and the method for outputting the interactive content corresponding to the interactive instruction, reference may be found in FIG. 9 and its related descriptions.

In some embodiments, the system for assisted fitness 600 may also include an evaluation module 650. The evaluation module 650 may be configured to obtain an interactive action of a user to be evaluated, the user to be evaluated being at least one user participating in interaction, evaluate an action standard degree of the interactive action, and obtain evaluation information of the user to be evaluated.

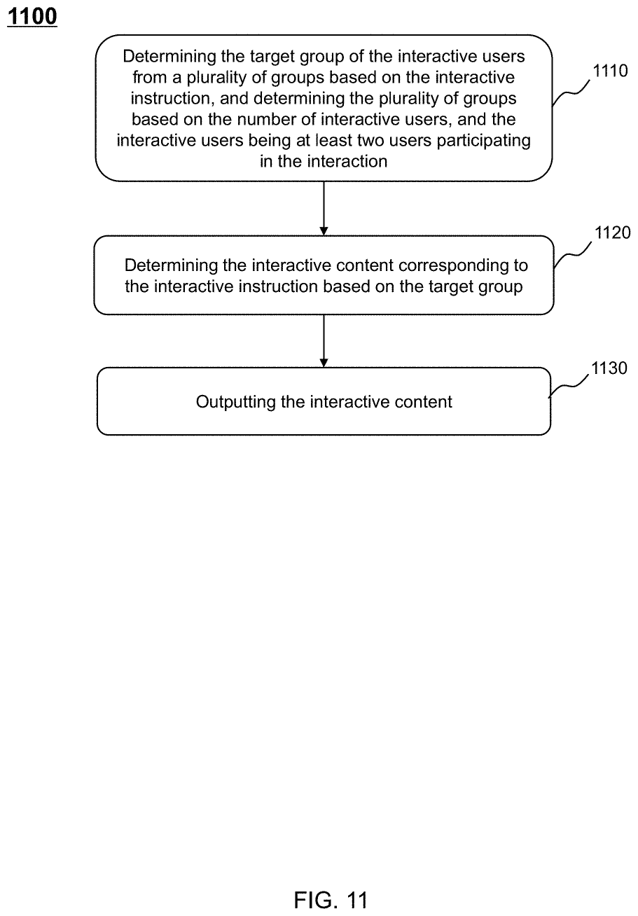

More descriptions of the definition and description of the user to be evaluated, the interactive action, the action standard degree and the evaluation information, and the method for obtaining the evaluation information of the user to be evaluated, reference may be found in FIG. 11 and its related descriptions.

It should be understood that the system and its modules shown in FIG. 6 may be implemented in various ways.

It should be noted that the above descriptions of the system for assisted fitness 600 and its modules is only for the convenience of description, and may not limit the present disclosure to the scope of the illustrated embodiments. It may be understood that for those skilled in the art, after understanding the principle of the system, various modules may be combined arbitrarily, or a subsystem may be formed to connect with other modules without departing from the principle. In some embodiments, the identification module 610, the obtaining module 620 and the execution module 630 disclosed in FIG. 6 may be different modules in a system, or may be one module to implement the functions of the above two or more modules. For example, each module may share one storage module, and each module may also have its own storage module. Such deformations are all within the protection scope of the present disclosure.

FIG. 7 is an exemplary flowchart of a method for assisted fitness according to some embodiments of the present disclosure. As shown in FIG. 7, the process 700 may include the following operations.

In operation 710, identifying at least one user using the fitness apparatus based on the collected data, and determining an identification result. In some embodiments, the operation 710 may be performed by identification module 610.

The collected data may refer to data related to user identification, such as image data, voice data, etc., weight data, or the like. In some embodiments, the collected data may be collected by a data collection apparatus (e.g., a camera, a microphone, a wireless communication apparatus, etc.).

In some embodiments, the collected data may include at least one of image data of at least one user, biological data of at least one user, terminal apparatus data carried by at least one user, and voice data of at least one user.

The image data may refer to data including an image of a user. In some embodiments, the image data may include face information of the user, a set of key points of the user (e.g., a set of action key points such as arms, legs, etc.), or the like. In some embodiments, the image data may be obtained by a camera. The identification module 610 may use, for example, face recognition technology to extract the face information in the image data. The identification module 610 may also extract a set of key points corresponding to the user in the image data by using, for example, a human body recognition technology.

The biometric data may refer to data reflecting a user's biometrics. The biometric data may include, but is not limited to, one or more of iris data, fingerprint data, and vein data. In some embodiments, the biometric data may be obtained by a biometric information collection apparatus in communication with the system for assisted fitness. For example, the fingerprint data may be obtained by a fingerprint sensor.

The terminal apparatus data may refer to the related data of the terminal apparatus used by the user. The terminal apparatus data may include unique identification information possessed by the terminal apparatus, for example, an identification code of a mobile phone. In some embodiments, the system for assisted fitness may establish a communication connection (e.g., Bluetooth, Wi-Fi, etc.) with the terminal apparatus carried by the user to obtain terminal apparatus data.

The voice data may refer to voice data uttered by a user. The voice data may include voiceprint information corresponding to the user. In some embodiments, the system for assisted fitness may obtain voice data through a microphone.

The collected data may also include other types of data related to the user, which is not limited here.

Some methods described in the embodiments of the present disclosure may collect one or more kinds of data to adapt to the usage habits and data collection scenarios of different users, so as to facilitate subsequent accurate identification of one or more users.

Fitness apparatus may refer to assisted apparatus for fitness. For example, fitness apparatus may include, but is not limited to, fitness mirrors, terminal apparatus with fitness functions (e.g., mobile phones, game consoles) and other apparatus. More descriptions of fitness apparatus may be found in FIGS. 1-3 and their related descriptions.

In some embodiments, there may be one or more users using the fitness apparatus. In some embodiments, a plurality of users may use the same fitness apparatus at the same time. For example, a plurality of students may use the same fitness apparatus for fitness at the same time. For another example, coaches and students may use the same fitness apparatus for teaching and fitness at the same time. More descriptions of the user, reference may be found in FIG. 1 and the related descriptions about the user below.

In some embodiments, the identification result may include identity information of each user, fitness information of the user, and permission information of the user. The identity information of a user may include name, age, gender, blood type, weight, body fat percentage of a user, or the like. In some embodiments, the identity information of a user may be entered by the user. More descriptions of the identity information of the user, reference may be found in the related descriptions of the user information below. The fitness information of a user may include most frequent fitness items, recent fitness items, average fitness time, or the like. In some embodiments, the fitness information of a user may be updated in real time by the system for assisted fitness according to the fitness situation of a user. More descriptions of the fitness information of a user, reference may be found in the related descriptions of the behavior information of a user below.

The permission information of the user may include whether the user has the usage permission of the current fitness apparatus, the time of the usage permission, or the like. In some embodiments, the identification module 610 may set different permissions for a plurality of users. In some embodiments, the identification module 610 may determine permissions based on the identity information of the user. For example, the identification module 610 may set different permissions for the coaches and the students. In some embodiments, the control instruction issued by the user with different permissions may be different, and the functions that may be used may be different. For example, the identification module 610 may set the coach to have the permission to set fitness guidance course to be completed for each student, and set the student to have the permission to score for the coach.

In some embodiments, the identification module 610 may set corresponding permissions for different users according to different situations of the users. For example, the identification module 610 may set the permission according to the action standard degree of the user's fitness action. The higher the action standard is, the higher the user's permission is. It should be understood that the higher the permission of the user is, the more types of functions that the user may use are. Further, if the standard degree of the user's fitness action reaches the preset requirement, the identification module 610 may set the user's permission as the coach's permission. More descriptions of the standard degree, reference may be found in the related descriptions of FIG. 11. For another example, the identification module 610 may set the permission according to the fitness frequency of the user. The higher the fitness frequency of the user is, the higher the permission of the user set by the identification module 610 is. For example, the identification module 610 may add permission for additional services or functions (e.g., rewarding more fitness guidance courses).

In some embodiments, the related data of the user (e.g., image data, biological data, carried terminal apparatus data and voice data, etc.) may be pre-entered into the system for assisted fitness, and form a one-to-one correspondence with the identification result. The identification module 610 may determine the corresponding user and the identification result from the system for assisted fitness according to the collected data.

For example, the identification module 610 may extract the face information in the image data according to the image data of the user using the face recognition technology, identify different users, and determine the corresponding user and the identification result from the system for assisted fitness. For example, the identification module 610 may determine a user with matching biometric data and an identification result from the system for assisted fitness according to the biometric data of the user. For another example, the identification module 610 may determine the user corresponding to the matching terminal apparatus and the identification result according to the terminal apparatus data carried by the user. For another example, the identification module may extract the voiceprint information in the voice data by using the voiceprint recognition technology according to the voice data of the user, identify different users, and determine the corresponding user and the identification result from the system for assisted fitness. In some embodiments, the identification module 610 may also set different user IDs for different users according to the identification results, so that different users may be quickly determined through the user IDs during the use process of the fitness apparatus.

It may be understood that the identification module 610 may determine whether one or more users are currently using the same fitness apparatus at the same time according to the number of users corresponding to the identification result.

In some embodiments, if the identification module 610 does not identify the user, it may issue prompt content. The prompt content may include prompting that there is no user information, prompting to create an account and enter related data, etc.

In operation 720, obtaining a control instruction. In some embodiments, the operation 720 may be performed by the obtaining module 620.

A control instruction may refer to an instruction for triggering the fitness apparatus to perform one or more functions. For example, the control instruction may be an instruction for setting system functions, or an instruction for triggering interaction between users, or the like. In some embodiments, the control instruction may be in the form of one or more of actions and sounds.

In some embodiments, the control instructions may be set in various ways. In some embodiments, the control instruction may be the default setting of the system. For example, the default control instruction for “viewing fitness records” may be to click the related function icon. In some embodiments, the control instruction may be set by the user. The user may select a certain control content in any system setting list and customize its corresponding preset action. When the user makes the preset action, it may be used as a control instruction to trigger the execution of the corresponding control content, and the preset action may be obtained by collecting real-time image data of the user. For example, the user may set the instruction to increase the volume in the system settings as: pointing up and holding for 2 seconds. More descriptions of the control instruction, reference may be found in FIG. 2, FIG. 3, and the related descriptions of the feedback of users below.

In some embodiments, the control instructions may include interactive instructions. The interactive instructions may include related instructions that are applied in scenarios where a plurality of people use the same apparatus and/or a plurality of people use a plurality of apparatus to interact. More descriptions of the interactive instructions, reference may be found in FIG. 9 and related descriptions.

In some embodiments, the obtaining module 620 may obtain a control instruction based on interaction between the user and the fitness apparatus.

In some embodiments, the obtaining module 620 may obtain the corresponding control instruction based on the user touching the corresponding function icon and/or area on the screen. For example, the user may directly click the “settings” icon on the screen, and the obtaining module 620 may determine that the control instruction is to enter the setting page.

In some embodiments, the obtaining module 620 may obtain the control instruction based on the preset action corresponding to the control instruction made by the user. For example, the preset action corresponding to the control instruction “increase the volume” may be set as “pointing the index finger up”. When the obtaining module 620 determines that the index finger of the current user is pointing up, it is determined that the control instruction is “increase the volume”.

In some embodiments, the obtaining module 620 may also obtain a control instruction based on user information, environmental information, corrective information, or the like. More descriptions of the user information, the environmental information, and the corrective information, reference may be found in the related descriptions of the above information below.

In some embodiments, at least one pointer and at least one function icon may be displayed on the display apparatus of the fitness apparatus, and at least one pointer may have a corresponding relationship with at least one user. In some embodiments, the control instruction may include an instruction to control at least one pointer.

A pointer may be used to trigger different control contents. For example, the pointer may be moved to the functional area “shut down” on the display apparatus, and when the pointer is clicked, the apparatus may be turned off. In some embodiments, the pointer may be controlled by the user, for example, the user may control the pointer to move, and may also control the pointer to select and trigger a certain function, or the like. More descriptions of the pointer, reference may be found in FIG. 4, FIG. 5, and related descriptions.

A function icon may refer to an icon used to trigger one or more functions of a fitness apparatus. The function icons may include application icons (e.g., music applications, camera applications, etc.), system setting icons (e.g., power on/off, network connection, etc.), or the like.

In some embodiments, each pointer may correspond to a user. The user may control the pointer for which there is a corresponding relationship. For example, the number of users currently using a fitness apparatus is one, and there is one pointer on the display apparatus. The user may establish a corresponding relationship with the pointer. For another example, the number of users currently using the fitness apparatus is two, and there are two pointers on the display apparatus. Currently two users may establish a corresponding relationship with a pointer respectively. For example, the obtaining module 620 may determine the pointer corresponding to the user according to the position of the user relative to the fitness apparatus. For example, the user M and the user N are currently using the apparatus for assisted fitness. The user M is located on the left side of the fitness apparatus, and the user N is located on the right side of the fitness apparatus. Then, the obtaining module 620 may determine that the user M has a corresponding relationship with the pointer displayed on the left side of the display apparatus, and the user N has a corresponding relationship with the pointer displayed on the right side of the display apparatus. For another example, the obtaining module 620 may determine the pointer corresponding to the user according to the time sequence in which the user performs the preset actions. For example, the user M and the user N are currently using the apparatus for assisted fitness. The user M and the user N may make preset actions (e.g., applauding). If the user N makes a preset action earlier than the user M, the obtaining module 620 may assign any one of the two pointers to the user N, and assign the other pointer to the user M. The users and pointers in the above embodiments are only exemplary expressions, and the number of users and pointers is not limited. The number of users and pointers may be increased to be more. The obtaining module 620 may also establish the corresponding relationship between the pointer and the user based on other various methods. For example, the obtaining module 620 may determine the pointer desired by the user through voiceprint extraction and semantic recognition based on the voice data, and establish a corresponding relationship between the pointer and the user, etc., which is not limited herein.

In some embodiments, the obtaining module 620 may determine that the control instruction includes an instruction to control at least one pointer. For example, an instruction for the user to control the corresponding pointer. More descriptions of the pointer control, reference may be found in FIG. 4, FIG. 5, and the related descriptions of the target user controlling the target pointer below.

In some embodiments, the obtaining module 620 may also obtain various forms of instructions based on various methods. For example, user's voice data may be collected, and control instructions may be obtained through semantic analysis, which is not limited here.

In operation 730, in response to the control instruction, executing the control content of the fitness apparatus corresponding to the control instruction based on the identification result. In some embodiments, the operation 730 may be performed by the execution module 630.

The control content may refer to one or more functions in the fitness apparatus triggered by the control instruction, for example, shutting down, playing music, following a fitness guidance course, evaluating a course, or the like. In some embodiments, a fitness guidance course may also be referred to as fitness course.

In some embodiments, the control content may include display content on a display apparatus of the fitness apparatus, the display content may include at least one of fitness guidance course and a fitness action image of the at least one user. More descriptions of the display content, reference may be found in FIG. 8 and its related descriptions.

In some embodiments, when the identification result is that a user uses the fitness apparatus, the execution module 630 may execute the corresponding control content for the fitness apparatus according to the control instruction issued by the user. For example, the user A may issue an instruction “pause the fitness guidance course”. Then, the execution module 630 may pause the current fitness guidance course according to the instruction.

In some embodiments, when the identification result is that a plurality of users use a fitness apparatus at the same time, the execution module 630 may execute the control content corresponding to the different instructions according to different control instruction issued by different users. In some embodiments, the execution module 630 may display the same display area for the plurality of users through the screen of the display apparatus of the fitness apparatus to execute the control content corresponding to the control instructions issued by the plurality of users. For example, the identification result is that the user B and the user C use fitness apparatus at the same time. The user B and the user C may use the same display area. If the user B issues an instruction “enlarge the view”, the execution module 630 may enlarge the display view of the same display area according to the instruction. If the user C issues an instruction “switch fitness guidance courses”, the execution module 630 may switch the fitness guidance course in the same display area according to the instruction. In some embodiments, when the control content corresponding to the control instructions issued by a plurality of users conflicts, the execution module 630 may determine the control instruction to be executed according to the user's permission information or the time sequence of issuing the control instructions. For example, the user B may issue an instruction “enlarge the view”, and the user C may issue an instruction “keep the current view”. The user B may have higher permissions than the user C. The execution module 630 may determine that the instruction “enlarge the view” issued by user B is a control instruction to be executed. For another example, the permissions of the user B and the user C are the same. If the time when the user C issues an instruction is earlier than the time when the user B issues an instruction, and the time difference between the time when the user C issues an instruction and the time when the user B issues an instruction is less than a preset time threshold (e.g., 1 second), the execution module 630 may determine that the first issued instruction is the control instruction to be executed. For example, it is determined that the instruction “keep the current view” issued by user C is the control command to be executed.

In some embodiments, the execution module 630 may display different display areas for a plurality of users through the screen of the display apparatus of the fitness apparatus to execute the control content corresponding to the control instruction issued by the plurality of users. For example, the identification result is that the user A and the user B use fitness apparatus at the same time. The user A and the user B may use different display areas. For example, if the user A makes a control instruction for pausing the fitness guidance course, the execution module 630 may pause the fitness guidance course in the display area displayed for the user A. If the user B makes a control instruction for exiting the current fitness guidance course, the execution module 630 may exit the fitness guidance course in the display area displayed for the user B. For another example, if the user A performs a preset action of increasing the volume of the earphone matched with the fitness apparatus to issue a control instruction, the execution module 630 may send a control instruction to the earphone carried by the user A for increasing the playback volume of the earphone. If the user B makes a control instruction for switching the fitness guidance course, the execution module 630 may switch the fitness guidance course in the display area displayed for the user B. More descriptions of how to determine the display area, reference may be found in FIG. 8 and the related descriptions of setting the display area below.

In some embodiments, in response to the control instruction, the execution module 630 may determine the target user based on the identification result. The target user may be a user who issues a control instruction among at least one user.

In some embodiments, the execution module 630 may determine the target user in various ways based on the identification result.

In some embodiments, the execution module 630 may determine that the user issuing the control instruction is the target user.

In some embodiments, the execution module 630 may determine the target user according to the obtained image data. For example, at least one user may perform a specified action of issuing a control instruction, the camera of the fitness apparatus obtains image data, and the execution module 630 uses human body recognition technology to extract a set of key points (e.g., a collection of action key points for arms, legs, etc.) corresponding to at least one user in the image data to determine the target user. For another example, the execution module 630 may perform similarity analysis between the action of at least one user in the image data and the specified action to determine the target user.

In some embodiments, the execution module 630 may determine the target user according to the obtained voice data. For example, the execution module 630 may take a user who speaks a preset specific sentence (e.g., “I am the target user”) as the target user.

In some embodiments, the execution module 630 may determine the target user according to the terminal apparatus carried by the user. For example, the fitness apparatus may establish communication with the terminal apparatus carried by the user, and different terminal apparatus may have their unique identifiers (e.g., the identification code of the mobile phone, etc.), and the execution module 630 may determine the target user by receiving the control instruction sent by the target user.

In some embodiments, when the identification result is that a user uses the fitness apparatus and the user issues a control instruction, the execution module 630 may determine the user as the target user. For example, the identification result is that the user A uses fitness apparatus. If the user A issues an instruction “play music”, then the execution module 630 may determine that the user A is the target user. In some embodiments, when the identification result is that a plurality of users use the fitness apparatus at the same time, and at least one of the plurality of users issues control instructions, the execution module 630 may determine the at least one user as the target user. For example, the identification result is that the user B, the user C, and the user D use fitness apparatus. If only the user B issues an instruction “play fitness guidance course”, then the execution module 630 may determine that the user B is the target user. If the user B issues an instruction “enlarge the view” and the user C issues an instruction “switch the fitness guidance course”, the execution module 630 may determine that both the user B and the user C are the target users.

In some embodiments, when the identification result is that a plurality of users use the fitness apparatus at the same time, the execution module 630 may set a preset area near the fitness apparatus, and determine the target user based on an overlapping degree of each of the plurality of users with the preset area. For example, the target user with the highest overlapping degree may be used as the target user. The execution module 630 may execute the control content corresponding to the control instruction issued by the target user.

The mode in which the execution module 630 determines the target user may include, but is not limited to, any one of the above methods and a combination thereof. The execution module 630 may also determine the target user in other ways, which is not limited here.

In some embodiments, the execution module 630 may determine the control content based on the function icon pointed by the target pointer, and the target pointer is a pointer corresponding to the target user. For example, when the target pointer points to the function icon corresponding to the music application, the execution module 630 may determine that the control content is to play music, and execute it. For another example, when the target pointer points to the function icon corresponding to “play fitness guidance course at 2x speed”, the execution module 630 may determine that the control content is to play fitness guidance course at 2x speed, and execute it. More descriptions of implementing control content through pointers, reference may be found in FIG. 4, FIG. 5, and their related descriptions.

In some embodiments, when there is a plurality of users, the display apparatus may display only one target pointer. The execution module 630 may determine a target user from a plurality of users, and control the target pointer based on the action of the target user at a specific key point to realize the control content. For example, the target user is the user D. The execution module 630 may determine that the wrist joint of the user D is a specific key point. The user D may control the action of the target pointer by moving the wrist joint, and may control the target pointer to select the function icon by stopping the wrist joint for a preset time (e.g., 2 seconds).

In some embodiments, when there are the plurality of target users, different target users may establish corresponding relationships with different target pointers. For more descriptions about establishing the corresponding relationship, reference may be found in the related descriptions of the foregoing operation 720. In some embodiments, the display apparatus may display different target pointers corresponding to different target users. For example, different pointers may be distinguished by different colors, shapes, texts, etc. For example, if the target users are user A and user B, the execution module 630 may determine that the pointer corresponding to user A is red, and the pointer corresponding to user B is green.

In some embodiments, different target users may control their corresponding pointers through actions of their respective specific key points to realize the control content. For example, if the target users are the user A and the user B. The execution module 630 may determine that the wrist joint of the user A is a specific key point, and the first of the user B is a specific key point. If the user A moves the corresponding red pointer by moving the wrist joint, and moves the red pointer to the function icon “increase the volume” to issue a control instruction “increase the volume”, the execution module 630 may increase the playback volume of the earphones carried by the user A. If the user B moves the corresponding green pointer by moving his fist, and moves the green pointer to the function icon “switch a fitness guidance course” to issue a control instruction “switch fitness guidance courses”, the execution module 630 may switch the fitness guidance course in the display area displayed for the user B.

Some methods described in the embodiments of the present disclosure control different pointers by different users, and perform corresponding control content by identifying the target user, so that the fitness apparatus may be applied to the scene used by many people, and the orderly use of the fitness apparatus can be ensured.

In some embodiments, the execution module 630 may determine a target user based on the control instruction, and the target user may be at least one user who issues the control instruction. More descriptions of the target user, reference may be found in the foregoing descriptions about determining the target user.

In some embodiments, the execution module 630 may determine the control content corresponding to the target user based on the target user and the control instruction, and execute the control content.

In some embodiments, the system for assisted fitness may pre-store feature sets of a plurality of preset control instructions of a plurality of users, and identify control instructions issued by different users based on the feature sets for execution. For example, the system for assisted fitness may store a feature set of preset control instructions corresponding to different users based on the user identification. The feature set may represent the features executed when different users issue preset control instructions, and the features may be image features or video features, key point features (e.g., key features of wrist and elbow joints), voiceprint features, etc. For example, if the preset control instruction is a voice instruction, the feature set may include voiceprint features when different users issue the preset control instruction. For another example, if the preset control instruction is an action instruction, the feature set may include image features, video features, or the like when different users issue the preset control instruction.

For example, the execution module 630 may determine that the target user is the user E, and the preset control instructions include: nodding—playing the latest fitness guidance course, shaking head—pausing the fitness guidance course. When the user E makes an action of shaking head, the execution module 630 may determine that the corresponding control content is to pause the fitness guidance course.

In some embodiments, when different users issue different control instructions or the same control instruction, the actual executed control contents may be different. For example, there may be differences in the preset actions set by the user, so different preset actions may correspond to executing the same control content, and the same preset action may also correspond to executing different control content. For example, the preset action corresponding to the control instruction of the user A for implementing “increase the volume” may be set to pointing index finger up, and the preset action corresponding to the control instruction of the user B for implementing “increase fitness intensity” may also be set to pointing index finger up. When the user A is the target user and the points index finger up, the execution module 630 may determine that the control content is “increase the volume”. When the user B is the target user and points index finger up, the execution module 630 may determine that the control content is “increase fitness intensity”. For another example, the user A and the user B may both set that pointing the finger up indicates increase the volume of the earphones. However, when performing the increase the volume on the earphone, the finger of the user A is 90 degrees upward relative to the ground, and the finger of the user B is 30 degrees upward relative to the ground.

Some of the methods described in the embodiments of the present disclosure implement personalized operation on the fitness apparatus by identifying the control instructions issued by different target users, so that the fitness apparatus may adapt to the usage requirements of more users.

In some embodiments, the execution module 630 may determine the display content and the display area based on the control instruction and the target user. More descriptions of the display content and the display area, reference may be found in FIG. 8 and its related descriptions.

Some methods described in the embodiments of the present disclosure execute the control content corresponding to the control instruction adapted to the current identification result, by identifying the user, which may adapt to the scene of a single person using the fitness apparatus or a plurality of people using the fitness apparatus at the same time. While meeting the individual requirements of users, it ensures the orderly operation of the apparatus and improves the interactivity and fun of fitness.

FIG. 8 is a schematic diagram illustrating executing the control content of the fitness apparatus corresponding to the control instruction according to some embodiments of the present disclosure.

In some embodiments, the control content may include display content 830 on a display apparatus of the fitness apparatus.

The display content 830 may refer to content displayed on a display apparatus of a fitness apparatus. In some embodiments, the displayed content 830 may include at least one of a fitness guidance course, a fitness action image of at least one user.

The fitness guidance course may refer to a course for guiding a user to exercise. The fitness guidance course may include, but is not limited to, a combination of one or more of the following: a fitness guidance course pre-recorded by a fitness coach and/or a fitness guidance course pre-made based on users' fitness requirements. For example, the fitness guidance course may include, but is not limited to, a combination of one or more of the following: yoga, aerobics, pace running, fat-burning exercises, planks, push-ups, or the like. In some embodiments, the fitness guidance course may be in various forms, including one or a combination of video, image, voice, text, or the like. More descriptions of the fitness guidance course, reference may be found in the related descriptions in the fitness guidance course section below.

The fitness action image may refer to an image of the fitness action performed by the user when performing fitness using the fitness apparatus. For example, images of yoga actions, images of aerobics actions, etc.

In some embodiments, in response to the control instruction 810, the control content of the control instruction corresponding to the fitness apparatus may be executed based on the identification result includes the following operations. A target user 820 may be determined based on the control instruction 810, and the target user 820 is a user who issues the control instruction 810 among the at least one user. The display content 830 may be determined based on the control instruction 810. The display area of the target user 820 in the display apparatus of the fitness apparatus may be determined based on the target user 820. The display content 830 may be displayed on the display apparatus based on the display area. In some embodiments, the aforementioned operations may be performed by the execution module 630.

More descriptions of the target user 820, reference may be found in FIG. 7 and its related descriptions.

In some embodiments, the execution module 630 may determine the target user 820 according to the image data, voice data of the user, terminal apparatus carried by the user, or the like. More descriptions of determining the target user 820, reference may be found in FIG. 7 and its related descriptions.

In some embodiments, the execution module 630 may identify the control instruction 810 issued by the target user 820 to determine the display content. The control instruction 810 may be in various forms, for example, an action instruction, a voice instruction, or the like.

In some embodiments, the execution module 630 may analyze the action instruction of the target user 820 through the image data to determine the display content 830. For example, a user raising a hand is to determine a yoga course as the display content 830. For another example, at least one pointer may be displayed on the display apparatus, different target users 820 may correspond to pointers of different colors, the execution module 630 may control the pointer by identifying actions of the specific key points of the target user 820, and the target user 820 may select the desired content by controlling the pointer to determine the display content 830.

In some embodiments, the execution module 630 may analyze the voice instruction of the target user 820 through the voice data to determine the display content 830. For example, the target user 820 may directly speak the content to be displayed to the fitness apparatus, and the execution module 630 may receive and analyze the voice data to determine the displayed content 830.

In some embodiments, the execution module 630 may determine the display content 830 based on different control instructions 810 adopted by different target users 820. For example, the user A may set the finger up to call up the menu bar as the display content 830, and the user B may set the finger up to determine the yoga course as the display content 830. For another example, the user A may set the finger up to determine the yoga course as the display content 830, and the user B may set to use the terminal apparatus to send “1” to the fitness apparatus to determine the yoga course as the display content 830. In some embodiments, the fitness apparatus may pre-store feature sets of a plurality of preset control instructions of a plurality of users, and identify control instructions 810 issued by different users based on the feature sets to determine display content 830. For example, the fitness apparatus may store a feature set of a preset control instruction corresponding to different users based on the user identification. The feature set may represent the executed features when different users issue preset control instructions, and the features may be image features or video features, key point features (e.g., key features of wrist and elbow joints), voiceprint features, or the like. For example, if the preset control instruction is a voice instruction, the feature set may include voiceprint features when different users issue the preset control instruction. For another example, if the preset control instruction is an action instruction, the feature set may include image features, video features, or the like when different users issue the preset control instruction.

The mode in which the execution module 630 determines the display content may include, but is not limited to, any one of the foregoing methods and a combination thereof, and the execution module 630 may also determine the display content 830 in other modes, which are not limited here.

The display apparatus of fitness apparatus may include one or more combinations of displays such as projection type, stereoscopic imaging type, light-emitting diodes, etc., mirror glass with a certain proportion of reflection and perspective, display screen, and apparatus using related 3D technologies such as holographic projection, air projection, etc. In some embodiments, the display apparatus of the fitness apparatus may be all used to display the display content 830, part of which may be used to display the display content 830, and the rest part of which is made into a mirror surface to facilitate the user to observe their own actions. More descriptions of the display apparatus, reference may be found in the descriptions of display hardware below.

The display area may refer to the area of the target user 820 on the display apparatus, for example, the areas A, B, C, and D shown in FIG. 8. In some embodiments, the display area may be a certain area of the display apparatus, or may be all areas of the entire display apparatus.

In some embodiments, the execution module 630 may determine the target user 820 by the above-mentioned image data, voice data or terminal apparatus carried by the user, and then determine the display area of the target user on the display apparatus of the fitness apparatus.

In some embodiments, the display apparatus of a fitness apparatus may be divided into a plurality of display areas, which respectively display different contents for different target users 820. In some embodiments, the execution module 630 may determine areas where different target users 820 are located, and determine the corresponding display area on the display apparatus of the fitness apparatus according to the area where each target user 820 is located. For example, the display areas may be divided and numbered for the display apparatus according to the number of target users 820, and then the corresponding display areas for different target users 820 may be determined. For another example, a display area (e.g., the target user 820 standing on the right side of the display apparatus is correspondingly divided into the display area on the right side of the display apparatus, or the target user 820 farther away from the display apparatus is correspondingly divided into a larger display area) more suitable for the target user 820 may be determined according to the relative relationship (e.g., distance, orientation, etc.) between the target user 820 and the display device.

In some embodiments, the display area may be determined by a display area determination model. The display area determination model may refer to a model used to determine the display area corresponding to the target user 820 on the display apparatus of the fitness apparatus.

In some embodiments, the input of the display area determination model may include features of the display area (e.g., number, location, area, etc.) and target user information (e.g., number of people, and distance and orientation relative to the display apparatus, etc.). The output of the display area determination model may include features (e.g., number, location, etc.) of the display area corresponding to the target user 820. For example, the orientation information of the target user 820 relative to the display apparatus may be processed based on the display area determination model to determine the number of the display area corresponding to the target user 820.

In some embodiments, the display area determination model may include various feasible neural network models, for example, convolutional neural networks (CNN), deep neural networks (DNN), or the like, or any combination thereof.

In some embodiments, the display area determination model may be trained separately based on historical data. In some embodiments, the display area determination model may be obtained by training based on a plurality of training samples and labels.

In some embodiments, the training samples may include the features of the sample display area and the information of the sample target users. The label may be the feature of the display area that matches the information of the target user. The training samples may be obtained based on historical data, and the labels of the training samples may be determined by manual labeling or automatic labeling. The above description is only an example, not a limitation, and the labels of the training data may be obtained in various ways.

In some embodiments, the execution module 630 may divide the display area on the display apparatus of the fitness apparatus to display content corresponding to the requirement of each target user according to the requirement of each target user 820.

The requirements may refer to the requirements of the target user 820 for the display content displayed by the fitness apparatus. For example, the requirement of the target user A may be playing a yoga course, and the requirement of the target user B may be calling up the menu bar.

In some embodiments, the requirement of the target user 820 may be determined by identifying the control instruction 810. In some embodiments, the requirement of the target user 820 may be captured by direct inputting on the fitness apparatus by the target user. In some embodiments, the requirement of the target user 820 may be determined based on the information of the target user. More descriptions of the user information, reference may be found in the related descriptions in the previous section about obtaining information. The mode of obtaining the requirement of the target user 820 may include, but is not limited to, any one of the foregoing methods and a combination thereof.

In some embodiments, the content displayed on the divided different display areas may be fitness guidance courses corresponding to the requirement of the target user 820. For example, the second area of the display apparatus of the fitness apparatus may display a squat course for the user A, and the second area may display a burpee course for the user B. More descriptions of determining fitness guidance courses for different target users 820, reference may be found in the related descriptions of fitness guidance courses below.

As shown in FIG. 8, the execution module 630 may receive the control instruction 810, determine the target user 820 and the display content 830 based on the control instruction 810, and determine the display area of the target user on the display apparatus of the fitness apparatus based on the target user 820. For example, the display area corresponding to the target user is area A, and the execution module 630 may adjust the display content to area A on the display apparatus accordingly to display the display content on the display apparatus based on the display area.

In the methods described in some embodiments of the present disclosure, the display content may be adjusted to the display area corresponding to the target user. In the case of the plurality of users using a fitness apparatus at the same time, the display content of each display area may be relatively independent, ensuring that each user is not disturbed by others when exercising, thereby improving the user's experience. In addition, by displaying the display content in different areas, a fitness apparatus may be used by the plurality of users at the same time, which reduces the footprint of the apparatus and effectively improves the utilization rate of the fitness place.

FIG. 9a is an exemplary flowchart of outputting interactive content corresponding to an interactive instruction according to some embodiments of the present disclosure. FIG. 9b is a schematic diagram illustrating at least two users interacting using the same fitness apparatus according to some embodiments of the present disclosure, and FIG. 9c is a schematic diagram illustrating at least two users interacting using a plurality of fitness apparatus according to some embodiments of the present disclosure.

In some embodiments, the system for assisted fitness 600 may further include an output module. As shown in FIG. 9a, the process 900 may include the following operations.

In operation 910, in response to the control instruction including an interactive instruction. In some embodiments, the operation 910 may be performed by an output module.

The interactive instruction may refer to a control instruction issued by the user to the fitness apparatus for triggering the interactive content in the fitness process, for example, a specified action instruction, a specified voice instruction, a control instruction sent by a terminal apparatus carried by the user, or the like.

In some embodiments, the interactive instruction may be at least one of an interactive request issued by at least one user and a preset interactive action made by at least one user.

The interactive request may refer to a request issued by the user to the fitness apparatus for triggering interactive content in the fitness process, for example, voice interactive request, text interactive request, etc.

The preset interactive action may refer to an action made by the user facing the fitness apparatus for triggering the interactive content in the fitness process, for example, raising the right hand, raising the left hand, etc. In some embodiments, the preset interactive action may be a default action of the system for assisted fitness, or may be a user-defined action.

The method described in some embodiments of the present disclosure, by sending an interactive request or making a preset a preset interactive action or other interactive instruction for triggering interactive content, may reduce the tedious operations for triggering interactive content, improve the efficiency and stability of triggering, and improve the user's experience.

In operation 920, outputting the interactive content corresponding to the interactive instruction, the interactive content including interactive fitness data. In some embodiments, the operation 920 may be performed by an output module.

The interactive content may refer to the content of the user-to-user interaction on the exercise using the fitness apparatus. For example, the plank battle between users through fitness apparatus, the two-person yoga between users through fitness apparatus, the mutual learning of aerobics actions between users through fitness apparatus, etc.

The interactive fitness data may refer to data used to represent interactive information between users during a user interactive fitness process. For example, the common historical interactive records of the user A and the user B. For another example, the fit of the user A and the user B in the interactive fitness process, and the number of mistakes made by each. For another example, the respective body motion states of the user A and the user B during the interactive fitness process.

The output module may determine interactive content in various ways. In some embodiments, the interactive content may be determined according to at least one of interactive instructions between users, the number of users interacting, the information of users participating in the interaction, or the like.

In some embodiments, the fitness apparatus may display interactive content corresponding to the number of people interacting. For example, for double users, the fitness apparatus may display a pair dance, and for five users, the fitness apparatus may display a group dance. In some embodiments, the number of people interacting may be determined in various ways. For example, the number of people interacting may be determined based on the times of the interactive request sent and/or answered (within a preset time). For another example, the number of people interacting may be determined based on the number of users who input the same verification information in the interactive content request interface (within a preset time).

In some embodiments, the output module may determine the current body movement state of the user according to the user information of the plurality of users, and display the interactive content corresponding to the body movement state of the plurality of users. More descriptions of the user information and obtaining the user information, reference may be found in the previous descriptions of obtaining information. The current body movement state of the user may include the acceptable exercise intensity of the user, the physical condition of the user, the active fitness status of the user, etc. For example, the output module may determine the acceptable exercise intensities of different users according to user information (e.g., exercise data and/or physiological data, etc.), and assign corresponding interactive content to corresponding users according to the exercise intensities of different users. For example, when the user E interacts with the user F in a pair dance, the fitness apparatus may guide the user E who may accept higher exercise intensity to perform the interactive action “guide”, and may guide the user F who may accept lower exercise intensity to perform interactive action “follow”.

In some embodiments, the output module may also determine the interactive content through other information, such as environmental information, or the like. More descriptions of other information, reference may be found in the related descriptions of obtaining information above.

In some embodiments, the output module may group a plurality of users participating in the interaction, and determine the corresponding interactive content for each user group. For example, if there are five users participating in the interaction, the system for assisted fitness may divide the users into two-person group and three-person group, and provide two-person interactive content for users of the two-person group and three-person interactive content for users of the three-person group.

In some embodiments, the output module may also determine the interactive content based on the interactive request, the preset interactive action etc., of the user. More descriptions of the interactive request and the preset interactive user action of the user, reference may be found in the related descriptions of identifying and outputting the interactive content below.

The mode of determining the interactive content may include, but is not limited to, any one of the foregoing methods and a combination thereof, and the interactive content may also be determined by other implementation modes, which are not limited here.

In some embodiments, the output module may determine and output the corresponding interactive content according to the interactive request between users and the feedback of the interactive request. For example, the user A may send a battle request to the user B, and if the user B accepts it, the system for assisted fitness may determine that the battle course may be used as the interactive content. In some embodiments, the interactive request may be implemented through request information sent by one user to one or more other users, or through the same authentication information entered by a plurality of users on a specific interface. The interactive request may also be implemented in other ways, which are not limited here.

In some embodiments, the output module may determine whether there is interaction between the users according to the preset interactive actions (e.g., holding hands, clapping shoulders) of the users, and then the fitness apparatus may display the interactive content corresponding to the preset interactive actions. In some embodiments, the preset interactive actions and their corresponding interactive contents may be preset and stored. For example, if two users hold each other's hands for 5 seconds, the fitness apparatus may display a video of a pair dance. For another example, if the two users touch each other's shoulders for 5 seconds, the fitness apparatus may play the guidance voice for the two people to press the shoulders.

In some embodiments, the output module may determine whether there is interaction between users according to the sets of key points of actions of a plurality of the user, and then the fitness apparatus may display the interactive contents corresponding to the preset interactive actions. For example, it is determined whether the overlapping area or the overlapping degree of the two sets of key points reaches the overlapping threshold. If the overlapping area or the overlapping degree of the two sets of key points reaches the overlapping threshold, it may be determined that there is interaction between users, and then when the maintenance time of the preset interactive actions between a plurality of users reaches a time threshold, it may be determined that interaction occurs between users. For example, if the overlapping degree (e.g., 65%) of the set of key points of the user C and the set of key points of the user D exceeds the overlapping threshold (e.g., 50%), the system for assisted fitness may determine that there is an interaction between the user C and the user D, and the maintenance time (7 seconds) of the interactive action exceeds the time threshold (5 seconds), it is determined that the interaction occurs between the user C and the user D.

In some embodiments, the output module may match the sets of key points of the plurality of users with the sets of key points of the preset interactive actions to determine whether there is interaction between the users, and then the fitness apparatus may display the interactive content corresponding preset interactive actions. For example, if the set of key points and the set of key points of the preset interactive action are successfully matched (e.g., the matching degree is greater than the threshold), it means that the preset interactive action occurs between the users, and then the interactive content corresponding to the preset interactive action may be selected. For example, if the set of key points of the user C and the set of key points of the user B are successfully matched with the set of key points of the preset two-person shoulder pressing action, the system for assisted fitness may determine that the user C and the user B have a two-person shoulder pressing action, and then the fitness apparatus may display a fitness video of the two-person shoulder pressing action.

In some embodiments, the output module may also use a preset interactive recognition model to identify whether there is interaction between users, and then the fitness apparatus may display the interactive content corresponding to the preset interactive action. The preset interactive recognition model may refer to a model for determining whether an interaction occurs between users.

In some embodiments, the input of the preset interactive recognition model may include the sets of key points of a plurality of users such as wrist, elbow, etc. The output of the preset interactive recognition model may include a determination result of whether interaction occurs between users, for example, interaction occurs between users and no interaction occurs between users.

In some embodiments, the preset interactive recognition model may include various feasible neural network models. For example, convolutional neural networks (CNN), deep neural networks (DNN), or the like, or any combination thereof.

In some embodiments, the preset interactive recognition models may be independently trained based on historical data. In some embodiments, the preset interactive recognition model may be obtained by training based on a plurality of training samples and labels.

In some embodiments, the training samples include a set of key points for sample users. The label is the identification result of determining whether there is interaction between users based on the set of key points of the user. The training samples may be obtained based on historical data, and the labels of the training samples may be determined by manual labeling or automatic labeling. The above description is only an example, not a limitation, and the labels of the training data may be obtained in various ways.

The mode of identifying whether there is interaction between users and outputting the interactive content may include but is not limited to any one of the above methods and their combination. Identifying and outputting interactive content may also be implemented in other ways, which are not limited here.

In some embodiments, in response to the control instruction including the interactive instruction, and at least two users corresponding to the interactive instruction using the same fitness apparatus, the interactive content corresponding to the interactive instruction may be outputted.

In some embodiments, a plurality of users may interact simultaneously using the same fitness apparatus. For example, a plurality of students may perform fitness on the same fitness apparatus at the same time. For another example, coaches and students may use the same fitness apparatus for teaching and fitness at the same time. The use of the same fitness apparatus by a plurality of users may also include other scenarios, for example, a plurality of students and a plurality of coaches use at the same time, etc., which may not be repeated here.

In some embodiments, when a plurality of users use the same fitness apparatus, a plurality of users may interact during the fitness. Correspondingly, the fitness apparatus may output interactive content related to the interaction. The interactive content may be fitness guidance courses such as fitness videos, voice guidance, and projection related to a plurality of users. For more descriptions, reference may be found in the related descriptions of the fitness guidance courses below. For example, two friends may interact with each other through fitness actions such as two-person shoulder pressing, two-person belly, and passing the ball, and the fitness apparatus may display the corresponding fitness video of the two-person fitness. For another example, a plurality of family members may interact through fitness actions such as group dance and aerobics, and the fitness apparatus may play the voice guidance of the corresponding group fitness actions.

In some embodiments, similar to displaying the fitness data of the user, the fitness apparatus may display interactive fitness data between a plurality of users.

In some embodiments, if corresponding content may be displayed for a plurality of users in different areas before a plurality of users interact, and after a plurality of users interact, the fitness apparatus may fuse the display areas of the interactive users to display the interactive fitness data, or interactive content related to the interaction (e.g., fitness guidance courses, etc.) of the user. The fusion means that the display areas corresponding to a plurality of users are combined into one display area for display.

FIG. 9b is a schematic diagram illustrating at least two users interacting using the same fitness apparatus according to some embodiments of the present disclosure. As shown in FIG. 9b, when the users A, B, C, and D use the same fitness apparatus 911, the users A and B want to interact, and in response to the interactive instruction of the users A and B, the interactive content corresponding to the interactive instruction may be displayed on the display areas A and B corresponding to the users A and B. Since the users A and B use the same fitness apparatus 911 to perform interactive fitness in the same fitness place, the display areas A and B may be fused into a same display area K, and the interactive content may be displayed in the same display area K.

In some embodiments, in response to the control instruction including an interactive instruction, and at least two users corresponding to the interactive instruction using a plurality of fitness apparatus, the interactive content corresponding to the interactive instruction may be output.

In some embodiments, the system for assisted fitness may contain a plurality of fitness apparatus, and the plurality of fitness apparatus may be located at the same place or at different places.

In some embodiments, a plurality of users may interact through different fitness apparatus of the same system for assisted fitness. For example, the user E and the user F may respectively use the fitness apparatus 1 and the fitness apparatus 2 for fitness independently, and the user E and the user F may also use the fitness apparatus 1 and the fitness apparatus 2 for fitness interactively. For another example, the user E and the user F may respectively use the fitness apparatus 1 and the fitness apparatus 2 to perform interactive fitness at the same place, and the user E and the user F may also respectively use fitness apparatus 1 and fitness apparatus 2 to perform interactive fitness at two different places.

In some embodiments, the output module may establish communication between the fitness apparatus and other fitness apparatus according to an interactive request sent by a fitness apparatus to other fitness apparatus, so that the fitness apparatus and other fitness apparatus may cooperate to provide interactive services for the plurality of users. In some embodiments, when a plurality of users interact, the fitness apparatus and other fitness apparatus may be synchronized, so that the fitness apparatus and other fitness apparatus may provide interactive content for the plurality of users as a whole.

In some embodiments, when a plurality of users interact, a plurality of fitness apparatus of the same system for assisted fitness may provide roaming services for the interactive users, so that when the plurality of users move from one fitness apparatus to another fitness apparatus, the fitness apparatus may provide the same interactive content to the plurality of users. For example, when the user E and the user F interact, if the user E and the user F move from the fitness apparatus 1 to the fitness apparatus 2, the interactive content provided by the fitness apparatus 1 may be transferred to the fitness apparatus 2 for displaying. For another example, the user E and the user F may use the fitness apparatus 1 to interact. If the user E moves from the fitness apparatus 1 to the fitness apparatus 2, the interactive content provided for the user E in the fitness apparatus 1 may be transferred to the fitness apparatus 2 for displaying.

In some embodiments, when a plurality of fitness apparatus of the same system for assisted fitness are located at different places, the fitness apparatus may collect the fitness data corresponding to the user and send the fitness data to other fitness apparatus, and other fitness apparatus may synchronously display the fitness data of the user so that other users may view the interaction. Further, the system for assisted fitness may integrate the fitness data of the users participating online, and then send the integrated fitness data package to the fitness apparatus participating online at the same time, which may reduce the delay caused by the online.

Similarly, in some embodiments, a plurality of users may also interact through a plurality of different system for assisted fitness. A plurality of the system for assisted fitness may be located at the same place or at different places. More descriptions of establishing communication and interacting among a plurality of the system for assisted fitness, reference may be found in the foregoing related descriptions of the plurality of fitness apparatus of the same system for assisted fitness.

FIG. 9c is a schematic diagram illustrating at least two users interacting using a plurality of fitness apparatus according to some embodiments of the present disclosure. As shown in FIG. 9c, when users A, B, C, D use the apparatus 912 for assisted fitness, and users 1, 2, 3, and 4 use fitness apparatus 913, the user A and the user 2 want to interact, in response to interactive instructions of the user A and the user 2, the interactive content corresponding to the interactive instruction may be displayed in the display area A corresponding to the user A and the display area 2 corresponding to the user 2, respectively.

In the method described in some embodiments of the present disclosure, the interaction of a plurality of users may be realized through the same fitness apparatus, which may reduce the floor space of the apparatus and improve the utilization rate of the fitness place, thereby providing users with spacious and comfortable fitness environment. The interaction of a plurality of users may be realized through a plurality of fitness apparatus, which may ensure that users may experience the interactive function even if the users are not in the same fitness place, which enhances the flexibility of the interaction.

In some embodiments, the output module may also be used to obtain interactive reference information, the interactive reference information includes at least one of the interactive modes between the interactive users, the number of interactive users, and the identification results of the interactive users. The interactive users may be at least two users participating in interaction. The interactive content corresponding to the interactive instruction may be determined based on the interactive reference information, and the interactive content may be outputted.

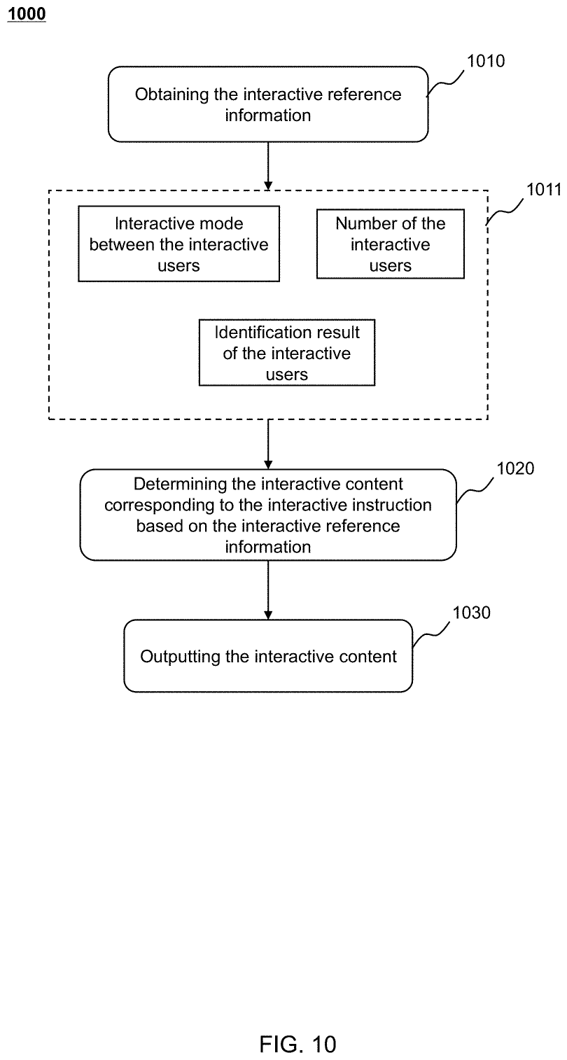

More descriptions of the interactive reference information, the obtaining mode of the interactive reference information, and the determination of the interactive content corresponding to the interactive instruction based on the interactive reference information, reference may be found in FIG. 10 and its related descriptions.

In some embodiments, the output module may also be used to determine a target group of the interactive users from a plurality of groups based on the interactive instruction, and the plurality of groups may be determined based on the number of interactive users. The interactive users are at least two users participating in the interaction. The interactive content corresponding to the interactive instruction may be determined based on the target group. The interactive content may be outputted.

More descriptions of determining the target group and determining the interactive content corresponding to the interactive instruction based on the target group, reference may be found in FIG. 11 and its related descriptions.

The method described in some embodiments of the present disclosure, through the interactive instruction outputting corresponding interactive content, may enhance the interaction between the users, strengthen the interest of fitness, and match more needs of users in the fitness process simultaneously.

FIG. 10 is an exemplary flowchart of determining interactive content according to some embodiments of the present disclosure. In some embodiments, the process 1000 may be performed by the system for assisted fitness. As shown in FIG. 10, the process 1000 may include the following operations.

In operation 1010, obtaining the interactive reference information.

The interactive reference information may refer to reference information for determining interactive content. As shown in FIG. 10, the interactive reference information 1011 may include at least one of the interactive modes between the interactive users, the number of the interactive users, and the identification result of the interactive users.

The interactive users may refer to users who need to communicate and interact during fitness. For example, the interactive users may include a plurality of fitness students, a plurality of fitness coaches, and one or more combinations of fitness coaches and fitness students.

The interactive mode of the interactive users may refer to the mode of communication and interaction between the interactive users or between the users and the fitness apparatus. For example, the interactive mode between the interactive user and the fitness apparatus may be that the user sends an instruction to the fitness apparatus through various modes, such as voice, gesture, and click, and the fitness apparatus feeds back related information to the user based on the instruction. For another example, the interactive mode between the interactive users may be information exchange, action interaction, etc., between users based on fitness apparatus.

In some embodiments, the interactive mode includes at least one of an interactive mode based on an interactive action and an interactive mode based on an interactive request. The interactive mode based on the interactive action may refer to the actual interactive action in the interaction process of the interactive user. For example, the interactive mode in the interaction process of the pair dance may be holding hands, holding up, moving back and forward, looking at each other, or the like. The interactive mode based on the interactive request may refer to the actual request in the interaction process of the interactive use. For example, the actual request sent by the user A to at least one other user, such as a racing request, a pair dance request, a follow-up request, and an action instruction request. More descriptions of the interactive request and interactive action, reference may be found in FIG. 9a and its related descriptions, which may not be repeated here.

The number of interactive users may refer to the number of users who currently want to participate in the interaction. In some embodiments, the number of interactive users may be one or a plurality of (e.g., 2, 3, 5, etc.) users.

The identification result of the interactive user may refer to the result obtained by identifying the interactive user based on the collected information. More descriptions of the identification result of the interactive user, reference may be found in FIG. 7 and its related descriptions, which may not be repeated here.

In some embodiments, the system for assisted fitness may obtain interactive reference information in various ways.

In some embodiments, the system for assisted fitness may obtain the interactive mode of the interactive user through the instructions and/or information issued by the user. For example, it may be determined that the interactive mode is an interactive request of the pair dance by interacting with the interaction information of the pair dance input/selected by the user on the fitness apparatus. In some embodiments, the system for assisted fitness may obtain the interactive mode of the interactive users by identifying the user actions. For example, the user A and the user B may perform punching actions respectively, and the system for assisted fitness may determine that the interaction between the user A and the user B is a punching confrontation.

In some embodiments, the interactive mode based on the interactive request may be obtained through request information sent by one user to one or more other users, or may be obtained through the same verification information input by the plurality of users on a specific interface. The interactive mode based on the interactive request may also be obtained in other ways, and more details are not described here.

In some embodiments, the system for assisted fitness may determine the number of interactive users in various ways. For example, the system for assisted fitness may determine the number of interactive users based on the times of the interactive request is sent and/or answered within a preset time. For another example, the system for assisted fitness may determine the number of interactive users based on the number of people who input the same verification information on the interactive content request interface within a preset time. In some embodiments, the number of interactive users may also be determined in other ways, which is not limited in the present disclosure.

In some embodiments, the system for assisted fitness may identify at least one user using the fitness apparatus based on the collected data to determine an identification result. More descriptions of determining the identification result of the interactive users, reference may be found in FIG. 7 and its related descriptions, which may not be repeated here.

In operation 1020, determining the interactive content corresponding to the interactive instruction based on the interactive reference information.

In some embodiments, the system for assisted fitness may determine the interactive content corresponding to the interactive instruction based on at least one or a combination of the reference information.

In some embodiments, the system for assisted fitness may determine the interactive content corresponding to the interactive instruction according to the interactive request of the interactive user. For example, the user A may send a battle request to the user B, and if the user B accepts the battle request, the system for assisted fitness may determine the battle course as the interactive content. For another example, if the user A and the user B input the verification code for the battle on the fitness apparatus at the same time, the system for assisted fitness may determine that the interactive content is a battle request.

In some embodiments, the system for assisted fitness may determine the interactive content by matching the interactive content corresponding to the preset interactive actions based on the interactive action between the interactive users. In some embodiments, the interactive content corresponding to the preset interactive action may be set and stored in advance by the system for assisted fitness, or may be set by the user according to preferences and habits. For example, the interactive action may be preset that two users hold hands for 5 seconds, and the corresponding interactive content is a pair dance. For another example, the interactive action may be preset that a plurality of users clap hands three times, and the corresponding interactive content is aerobics. In some embodiments, more interactive actions and corresponding interactive contents may be preset, which is not limited in the present disclosure.

In some embodiments, the system for assisted fitness may determine interactive content based on the number of interactive users. In some embodiments, the fitness apparatus may determine at least one interactive content for selection by the user based on the number of interactive users. More descriptions of determining the interactive content based on the number of interactive users, reference may be found in FIG. 9 and its related descriptions, which may not be repeated here.

In some embodiments, the system for assisted fitness may determine the interactive content according to the identification result of the interaction. In some embodiments, the system for assisted fitness may determine the interactive content based on the user information in the identification result of the interaction. In some embodiments, the system for assisted fitness may determine the current state of the user according to the user information of the plurality of users, and display interactive content corresponding to the states of the plurality of user. More descriptions of determining the interactive content based on the identification result of the interaction, reference may be found in FIG. 9 and its related descriptions.

In some embodiments, the system for assisted fitness may also determine the interactive content in other ways, which is not limited in the present disclosure. For example, the system for assisted fitness may determine the interactive content based on environmental information, the popularity of the interactive content, etc. More descriptions of the obtaining environmental information, user information, and the popularity of the interactive content, reference may be found in the related descriptions in the section about obtaining information, which may not be repeated here.

In operation 1030, outputting an interactive content.

In some embodiments, the system for assisted fitness may output interactive content to the user through the fitness apparatus corresponding to the user. For example, the system for assisted fitness may determine that the interactive content is a pair dance for the user A and the user B. If the two users use the same fitness apparatus, the interactive content may be outputted through the fitness apparatus. If the two users use different fitness apparatus, the interactive content may be outputted through the corresponding fitness apparatus. In some embodiments, the system for assisted fitness may output interactive content to the user through a display apparatus on the fitness apparatus. For example, the interactive content may be output to the user through a display screen, a display, or the like.

In some embodiments, interactive content may be displayed in the entire display area of the display apparatus when the interactive users use the same fitness apparatus. In some embodiments, when the interactive users use the same fitness apparatus and the interactive content between the users is different, the display area of the display apparatus may be divided to display different interactive contents in different areas. For example, assuming that the interactive content is a male and female pair dance, and there are differences between the dance action of male users and female users, partition display may be performed on the display apparatus, so that users may see more clearly.

More descriptions of outputting interactive content, reference may be found in FIG. 9 and its related descriptions, which may not be repeated here.

In the methods described in some embodiments of the present disclosure, a plurality of interactive reference information is obtained in various ways, and a plurality of interactive contents is determined for the interactive user based on the different interactive reference information, which may provide rich interactive fitness content for various combinations of interactive users, and determine the interactive content in various and flexible ways. In addition, the method may automatically determine the interactive content that is suitable for the user and the user likes based on the actual situation and interactive actions of the interactive users, which allows the user to select interactive fitness content more efficiently and conveniently, and improves the user's enthusiasm for fitness.

FIG. 11 is an exemplary flowchart of determining interactive content according to some embodiments of the present disclosure. In some embodiments, the process 1100 may be performed by a processor. As shown in FIG. 11, the process 1100 may include the following operations.

In operation 1110, determining the target group of the interactive users from a plurality of groups based on the interactive instruction, and determining the plurality of groups based on the number of interactive users, and the interactive users being at least two users participating in the interaction.

A plurality of groups may refer to groups formed by dividing interactive users according to a certain rule. In some embodiments, the system for assisted fitness may divide the interactive users based on various division rules to form a plurality of groups. For example, the division rule may be the number of the interactive user, the physical ability of the interactive user, the identity of the interactive user, the physical health status of the interactive user, or the like. For example, the interactive users may be divided into 2-person groups, 3-person groups, etc. based on the number of the interactive users. The interactive users may be divided into the coach group and the student group based on the identity of the interactive users. The interactive users may be divided into different groups according to the parts that students need to exercise based on physical health status of the interactive user.

In some embodiments, the system for assisted fitness may also determine a plurality of groups for the interactive students based on other methods, which are not limited in the present disclosure.

The target group may refer to the group to which each user of the interactive users belongs. For example, the target group may be a 2-person group, a 3-person group, or the like. For another example, the target group may also be a coach group, a student group, etc., which is not limited in the present disclosure.

In some embodiments, the system for assisted fitness may determine a target group of interactive users from a plurality of groups based on the actual instruction of the interactive instruction. In some embodiments, the actual instruction of the interactive instruction may be an instruction based on the interactive content, an instruction based on the physical ability of the interactive user, an instruction based on the identity of the interactive user, an instruction based on the age of the interactive user, or the like.

In some embodiments, the interactive users may be divided into target groups corresponding to different interactive contents based on the interactive contents. For example, when the interactive content is a group dance, and different users in the group dance may need to perform different action interactions, the interactive instruction may be dividing the interactive users into a plurality of groups based on different actions in the group dance, and then the target group of each interactive user may be determined according to the historical proficiency of the interactive user for corresponding actions in each group, etc.

In some embodiments, the interactive users may be divided into a plurality of target groups with comparable physical ability based on the physical ability of the interactive users, and the physical ability of the interactive users in each target group is equivalent. For example, when different users of the interactive users have different physical abilities, the interactive instruction may be determining corresponding target groups based on the physical abilities of the interactive users.

In some embodiments, the interactive users may be divided into different target groups according to the identity categories based on the identities of the interactive users, and then the corresponding target groups may be determined based on the corresponding identities of each user, such as coach group, student group, etc.

In some embodiments, the interactive users may be divided into different target groups according to the age groups based on the age of the interactive users. For example, when the interactive content includes interactive parts corresponding to users of different age groups, the interactive instruction may be determining a target group corresponding to the age of each interactive user based on age information of the interactive user.

In some embodiments, the system for assisted fitness may also determine the target group in other ways based on the interactive instruction, which is not limited in the present disclosure.

In operation 1120, determining the interactive content corresponding to the interactive instruction based on the target group.

In some embodiments, the system for assisted fitness may determine the interactive content corresponding to the interactive instruction in various ways based on the target group.

In some embodiments, the system for assisted fitness may determine the interactive content for each interactive user based on the interactive content corresponding to the target group. For example, if the overall interactive content is male and female pair dance, the target group corresponding to the user A is the boy group, and the target group corresponding to the user B is the girl group, the system for assisted fitness may determine the interactive content corresponding to the boy group as the interactive content of the user A, and the interactive content corresponding to the girl group as the interactive content of the user B. For another example, the overall interactive content requires 5 people to participate together, some of the interactive content requires 3 people to interact, and the other part of the interactive content requires 2 people to interact, then the system for assisted fitness may divide the interactive users into a 3-person group and a 2-person group. The interactive content corresponding to the 3-person group may be determined as the interactive content of each interactive user in the 3-person group, and the interactive content corresponding to the 2-person group may be determined as the interactive content of each interactive user in the 2-person group.

In some embodiments, the system for assisted fitness may also determine the interactive content corresponding to the interactive instruction in other ways, which is not limited in the present disclosure.

In operation 1130, outputting interactive content.

In some embodiments, the system for assisted fitness may display interactive content to the user based on a display apparatus in the fitness apparatus. In some embodiments, the system for assisted fitness may output corresponding interactive content to the interactive users in each target group through the display apparatus based on the target group. For example, interactive content may be output based on the number of target groups, the number of people in each target group, or the like.

More descriptions of outputting or displaying interactive content, reference may be found in the descriptions of the display section, which may not be repeated here.

In the methods described in some embodiments of the present disclosure, the interactive users may be grouped based on the interactive content, an appropriate group may be determined for each interactive user based on the overall interactive content and the actual situation (e.g., physical ability, age, etc.) of each interactive user, and the interactive content of each interactive user determine may be determined based on the group, which may determine more appropriate interactive content for each interactive user to achieve a better fitness effect.

In some embodiments, the system for assisted fitness may obtain the interactive actions of the user to be evaluated, evaluate the action standard degree of the interactive actions, and obtain the evaluation information of the user to be evaluated.

The user to be evaluated may refer to a user who is needed to perform an evaluation of the action standard degree. For example, the user to be evaluated may be at least one of the interactive users.

In some embodiments, the system for assisted fitness may obtain the interactive actions of the user to be evaluated through a camera apparatus on the fitness apparatus. In some embodiments, the camera apparatus on the system for assisted fitness may capture interactive images or videos of the user to be evaluated, and obtain the interactive actions of the user through image recognition, or the like. For example, after obtaining the interactive images or videos of the user, an image recognition model may be used to extract the set of key point of each user action of the user, and then the interactive user action of the user may be generated according to the set of the key point.

The action standard degree may refer to the degree to which the interactive action of the interactive user matches the standard action. For example, the standard degree may be that the degree to which the interactive action of the interactive user matches the standard action is 50%, 80%, 90%, etc. In some embodiments, the system for assisted fitness may compare the interactive action of the interactive user with the standard action to determine the action standard degree.

The evaluation information may refer to the result obtained by evaluating the interactive action. In some embodiments, the evaluation information may be an evaluation score, an evaluation grade, or the like. For example, the evaluation score may be 60 points, 80 points, 90 points, etc., and the evaluation grade may be non-standard, relatively standard, very standard, or the like. In some embodiments, the evaluation information may be the difference between the interactive action of the user action and the standard action. For example, the interactive user action of the user is arm raise, but the actual arm raise angle of the user differs from the standard arm raise angle by 10°, it may be obtained that the evaluation information may be the arm raise angle differed by 10°. For another example, the evaluation information may also be the difference in elbow bending, the difference in leg bending, the difference in arm elevation, the difference in leg elevation, the difference in the number of rotations, or the like.

In some embodiments, the system for assisted fitness may use various methods to evaluate the standard degree of the interactive action to obtain evaluation information of the user to be evaluated. In some embodiments, the system for assisted fitness may determine the evaluation information based on the action standard degree of the interactive action. For example, the system for assisted fitness may preset an evaluation score for each interactive action of the action standard degree, and after determining the action standard degree of the interactive action, the corresponding evaluation score may be determined. For another example, the system for assisted fitness may preset an evaluation grade for an interactive action within a certain range of the action standard degree, and after determining the action standard degree of the interactive action, the corresponding evaluation grade may be obtained. For example, the system for assisted fitness may preset the evaluation grade of the interactive action within the range of the action standard degree below 60% as non-standard, the range of the action standard degree between 60% and 80% as a relatively standard, and the range of the action standard degree between 80% and 100% as a very standard. In some embodiments, the system for assisted fitness may also obtain evaluation information in other ways, which are not limited in the present disclosure.

In some embodiments of the present disclosure, by evaluating the interactive actions of the interactive users, the evaluation information of the standard degree of the interactive actions of the interactive users may be obtained, which may determine in time whether the user interactive actions are standardized and standard, and provide strong support for subsequent guidance and correction of user actions based on evaluation information.

In some embodiments, the system for assisted fitness may also determine fitness guidance information of the user to be evaluated based on the evaluation information.

The fitness guidance information may refer to the information that guides and corrects the interactive action of the interactive user in the fitness process. For example, the fitness guidance information may be raising the arm by 5°, bending the elbow by 5°, and squatting down by 5 cm. For another example, the fitness guidance information may be feedback and prompt information for the interactive user.

In some embodiments, the system for assisted fitness may determine fitness guidance information based on the difference between the interactive action and the standard action in the evaluation information of the interactive user. For example, the system for assisted fitness may determine the guidance information as raising the arm again by 5° based on the evaluation information being the arm raise differed by 5°. In some embodiments, the system for assisted fitness may determine the fitness guidance information based on the evaluation information being an evaluation score, or an evaluation grade. For example, the system for assisted fitness may determine the guidance information as “the current interactive action score is 70 points, please refer to the standard action for correction”, or “the evaluation grade of the current interactive action is the relatively standard, please continue to refer to the standard action for correction”, etc., based on the evaluation score as 70 points, or the evaluation level as the relatively standard, etc. More descriptions of determining fitness guidance information, reference may be found in the related descriptions of the corrective information section, which may not be repeated here.

In some embodiments, the system for assisted fitness may provide corrective information to the user based on the evaluation information of the interactive user to correct the fitness actions of the user. For example, when the breathing frequency of the user is incorrect during fitness, the user may be provided with a demonstration video of correct breathing frequency, etc., to correct the breathing frequency of the user.

In some embodiments, the system for assisted fitness may determine a temporary coach at the fitness site based on the evaluation information of the interactive user. For example, the interactive user with the best evaluation result among the interactive users may be determined as the temporary coach on site based on the evaluation information.

In some embodiments, the system for assisted fitness may also provide the interactive user with more fitness advice, fitness guidance, etc. based on the evaluation information of the interactive user, which is not limited in the present disclosure.

In some embodiments of the present disclosure, the fitness guidance information may be determined based on the evaluation information of the interactive actions of the interactive users, which may guide and correct the non-standard actions of the user in time, and improve the accuracy of the interactive actions of the user, improve the effect of exercise.

More descriptions of obtaining information in fitness apparatus, reference may be found in the following descriptions.

In some embodiments, during the operation of the fitness apparatus, the system for assisted fitness may assist the user to fitness based on the obtained information. The obtained information may refer to various information obtained by the system for assisted fitness. For example, the physiological parameter information (such as heart rate, blood pressure, etc.), fitness environment information (such as temperature, humidity), etc. of the user. For example, the system for assisted fitness may determine a fitness guidance course for the user based on the obtained information, so that the user may follow the fitness guidance course to perform fitness. More descriptions of determining the fitness course based on the obtained information, reference may be found in the related descriptions of the fitness course below. For another example, the system for assisted fitness may monitor the physical ability state of the user during the fitness based on the obtained information, and adjust the fitness guidance course adaptively according to the physical ability state of the user, so that the adjusted fitness guidance course matches with the physical ability state of the user. More descriptions of adjusting the fitness guidance course based on the obtained information, reference may be found in the related descriptions of the corrective information section.

In some embodiments, the obtained information may include user information, environmental information, or the like.

The user information may refer to information used to represent attributes and/or features of a user. The attributes and/or features of the user may include the identity of the user, the physiological parameters of the user, fitness behavior features, features that affect the fitness behavior of the user, or the like.

In some embodiments, the system for assisted fitness may store the obtained user information in a user information database. The user information database may be deployed in the fitness apparatus, server and/or storage apparatus in the system for assisted fitness. The server or storage apparatus may be in communication with the fitness apparatus.

In some embodiments, before obtaining the user information, the system for assisted fitness may request the user's authorization to allow the system for assisted fitness to collect the user information for assisting the user to perform fitness exercises.

In some embodiments, the system for assisted fitness may differentiate the users into unfamiliar users and familiar users based on whether there are user information corresponding to the users in the user information database. An unfamiliar user may refer to a user whose corresponding user information does not exist in the user information database, and a familiar user may refer to a user whose corresponding user information exists in the user information database. In some embodiments, when the system for assisted fitness identify that a certain user is an unfamiliar user, a corresponding user ID (e.g., user identification) may be established for the unfamiliar user, and stored in the user information database, and the unfamiliar user may change to a familiar user. After the user information of the user is subsequently collected, the collected user information may be bound with the user ID of the user, and stored in the user information database. The user ID may include, but is not limited to, a combination of one or more of the following: character ID, face ID, voiceprint ID, iris ID, fingerprint ID, real-name number ID (e.g., identity card number, passport number), and apparatus ID (e.g., the identification code of the mobile phone carried by the user).

In some embodiments, the user information may include a combination of one or more of user description information, the user behavior information, and the user association information.

The user description information may refer to information used to describe basic attributes and/or basic features of the user. The user description information may include: personally identifiable information, basic physiological parameters, and other information.

The personally identifiable information may refer to information used to represent a user's personal identity. The personally identifiable information may include, but is not limited to, a combination of one or more of the following: user ID, name, gender, date of birth, blood type, address (or mailing address), education, etc.

The personally identifiable information may be obtained in various ways. In some embodiments, the system for assisted fitness may obtain personally identifiable information through user input. In some embodiments, the system for assisted fitness may obtain personally identifiable information through identification. For example, the system for assisted fitness may identify the identity document of the user to obtain the personally identifiable information of the user. For example, the system for assisted fitness may identify the image of the certificate, and may also perform radio frequency identification on the ID card with built-in IC chip based on the radio frequency identification technology. In some embodiments, the system for assisted fitness may obtain personally identifiable information from a third-party service platform. For example, the system for assisted fitness may establish a communication connection with the third-party service platform, and the system for assisted fitness may log in through the login account of the third-party service platform after obtaining the authorization of the user, so that the system for assisted fitness may obtain the personally identifiable information of the user from the third-party service platform.

The personally identifiable information may also be obtained in other ways, for example, the personally identifiable information may be obtained through behavior data of the user browsing and using functional applications in the system for assisted fitness, which is not limited here. In some embodiments, after obtaining the personally identifiable information of the user, the system for assisted fitness may monitor changes in the personally identifiable information of the user in real time (or periodically) through a third-party apparatus, and update the personally identifiable information in the user information database.

The basic physiological parameters refer to some parameters that describe the basic physical state of the user. The basic physiological parameters may include, but are not limited to, a combination of one or more of the following: age, gender, height, weight, body fat rate, initial heart rate, initial heartbeat, or the like. The initial heart rate and initial heartbeat may refer to heart rate and heartbeat when not exercising.

In some embodiments, the system for assisted fitness may obtain basic physiological parameters in a mode similar to the mode of obtaining personally identifiable information. For example, the system for assisted fitness may obtain basic physiological parameters through user input or from a third-party service platform.

In some embodiments, the system for assisted fitness may identify and determine the basic physiological parameters of the user based on the image of the user. The image of the user may be input by the user, or may be obtained by the system for assisted fitness from other terminal apparatus (e.g., the user's mobile communication apparatus or smart wearable apparatus, etc.) of the user and/or collected by taking pictures of the user through a camera carried in the system for assisted fitness. More descriptions of the camera of the fitness apparatus and obtaining the image of the user based on the camera, reference may be the following descriptions.

In some embodiments, the system for assisted fitness may obtain basic physiological parameters from the third-party apparatus of the user. The third-party apparatus may include, but is not limited to, a combination of one or more of the following: a smart wearable apparatus (e.g., a smart bracelet, a smart watch and/or a smart headband, etc.), a mobile communication apparatus (e.g., a mobile phone and/or a tablet, etc.), and/or a desktop apparatus (e.g., a desktop computer, etc.), etc.

In some embodiments, after obtaining the basic physiological parameters of the user, the system for assisted fitness may monitor the changes of the basic physiological parameters of the user in real time (or periodically) through the third-party apparatus and update the basic physiological parameters in the user information database.

The user description information may also include other information that may reflect the user attributes or features, such as the user body contour information, or the like. The mode of obtaining the user description type information may include, but is not limited to, any one of the foregoing methods and a combination thereof. The system for assisted fitness may also obtain the user description information in other ways, which is not limited here.

The user behavior information may refer to related information that reflects the user's fitness behavior. The user behavior information may include, but is not limited to, a combination of one or more of the following: used fitness guidance courses, exercise images, used fitness apparatus, exercise index parameters, exercise physiological parameters, or the like.

The used fitness guidance course may refer to the fitness guidance course used by the user in the fitness process. The used fitness guidance course may be the fitness guidance course selected or subscribed to from all fitness guidance courses (that is, all fitness guidance courses in the course database). The fitness guidance courses may include, but ais not limited to, a combination of one or more of the following: a fitness guidance course pre-recorded by a fitness coach, a fitness guidance course pre-made based on the fitness requirement of users, or the like. For example, the fitness guidance course may include, but is not limited to, a combination of one or more of the following: yoga, an aerobic, pace running, fat-burning exercises, a plank, a push-up, or the like. More descriptions may be found in the related descriptions of the fitness guidance course section below.

In some embodiments, the system for assisted fitness may recommend at least one fitness guidance course for the user to select. For example, the system for assisted fitness may show recommended fitness guidance course on the display apparatus. For another example, the system for assisted fitness may scroll and play the recommended fitness guidance course on the display apparatus. For another example, the system for assisted fitness may voice broadcast the recommended fitness guidance course. There may be various ways for the user to select, for example, touching and clicking, voice selection, gesture selection, eye tracking, or the like.

The exercise image may refer to the image data collected by photographing the user while the user in fitness.

In some embodiments, the user in fitness is photographed based on the camera (or a camera module) to collect exercise images, the camera (or a camera module) may include, but is not limited to, a combination of one or more of the following: wide-angle camera, ultra-wide-angle camera, telephoto camera, infrared camera, etc. In some embodiments, the system for assisted fitness may detect the position of the portrait within the field of view of the infrared camera, the distance between the portrait and the camera module, and/or the distance between the portrait and the fitness apparatus based on the images captured by the infrared camera to select a camera with a suitable field of view for obtaining image. When it is detected that the positions of the portraits within the field of view of the infrared camera are all within the field of view of the wide-angle camera, the system for assisted fitness may enable the wide-angle camera to collect exercise images. When it is detected that the position of the portraits within the field of view of the infrared camera is beyond the field of view of the wide-angle camera, the system for assisted fitness may enable the ultra-wide-angle camera to collect exercise images. When it is detected that the distance between the portrait and the fitness apparatus is greater than a preset threshold, the system for assisted fitness may enable the telephoto camera to collect exercise images.

In some embodiments, the fitness apparatus may determine the distance between the portrait and the fitness apparatus within the field of view of the infrared camera based on the image captured by the infrared camera. The wide-angle camera, ultra-wide-angle camera and/or telephoto camera automatically focus on the portrait within the field of view based on the distance to capture a exercise image with depth of field.

In some embodiments, the camera (or a camera module) may be built into the fitness apparatus, e.g., a built-in camera (or a camera module).

In some embodiments, the camera (or a camera module) may be independent of the system for assisted fitness, that is, an external camera (or a camera module), the external camera (or a camera module) is connected in communication with the system for assisted fitness, and the external camera (or a camera module) may transmit the collected exercise images to the system for assisted fitness. In some optional implementations, the external camera (or a camera module) may be arranged in a preset fitness place, and when the user is exercising in the fitness place, the external camera (or a camera module) may capture the user's exercise image by shooting.

In some embodiments, the system for assisted fitness may preset the mapping relationship between the fitness guidance course and the opening of the internal/external camera (or a camera module) according to the specific fitness item in the fitness guidance course. For example, if the range of activities of the fitness items (e.g., solo jumping) in the fitness guidance course is small, and the built-in camera (or a camera module) may capture the complete exercise image of the user, the system for assisted fitness may turn on the built-in camera (or a camera module). When the user completes the above-mentioned fitness items with a small range of activities in a designated fitness place, the system for assisted fitness may also turn on an external camera (or a camera module). For another example, the fitness program in the fitness guidance course has a wide range of activities and a large number of users (e.g., multi-group aerobics) who are exercising at the same time, and the built-in camera (or a camera module) cannot capture the complete exercise images of all users, then the system for assisted fitness may turn on the external camera (or a camera module). For another example, if the fitness items in the fitness guidance course require the system for assisted fitness (or a single fitness apparatus) to follow the user over a long distance (e.g., outdoor running), the system for assisted fitness may turn on the built-in camera (or a camera module) to capture the user's body image in real time, and follow the user in real-time based on the captured user's body image.

In some embodiments, when the user is exercising, the system for assisted fitness may detect the position of the human body image in the field of view through the built-in camera (or a camera module), and output corresponding prompts based on the detection result. For example, when it is detected that the distance between the user and the fitness apparatus exceeds a preset maximum threshold, making the built-in camera (or a camera module) difficult to focus, the system for assisted fitness may prompt the user to get closer. When it is detected that the distance between the user and the fitness apparatus is less than the preset minimum threshold, making an incomplete human body image captured by the built-in camera (or a camera module), the system for assisted fitness may prompt the user to stay away. For another example, when it is detected that the user is facing the fitness apparatus and gradually moves out of the field of view of the built-in camera (or a camera module) from the left side of the fitness apparatus (or the user is facing the fitness apparatus and standing on the left of the center of the field of view) the system for assisted fitness may prompt the user to move a little to the right. When it is detected that the user is facing the fitness apparatus and gradually moves out of the field of view of the built-in camera (or a camera module) from the right side of the fitness apparatus (or the user is facing the fitness apparatus and stands on the right side of the center of the field of view), the system for assisted fitness may prompt the user to move a little to the left.

In some embodiments, when the system for assisted fitness still does not detect the user in the field of view of the built-in camera (or a camera module) within the preset time period (e.g., 10 seconds) after outputting the corresponding prompt, then the user following function may be started to find the user and follow the user. In some embodiments, the system for assisted fitness may stop the user following function until the user is detected within the field of view of the built-in camera when the system for assisted fitness does not detect the user within the field of view of the built-in camera (or a camera module) within a preset time period (e.g., 10 seconds) after the user following function is started.

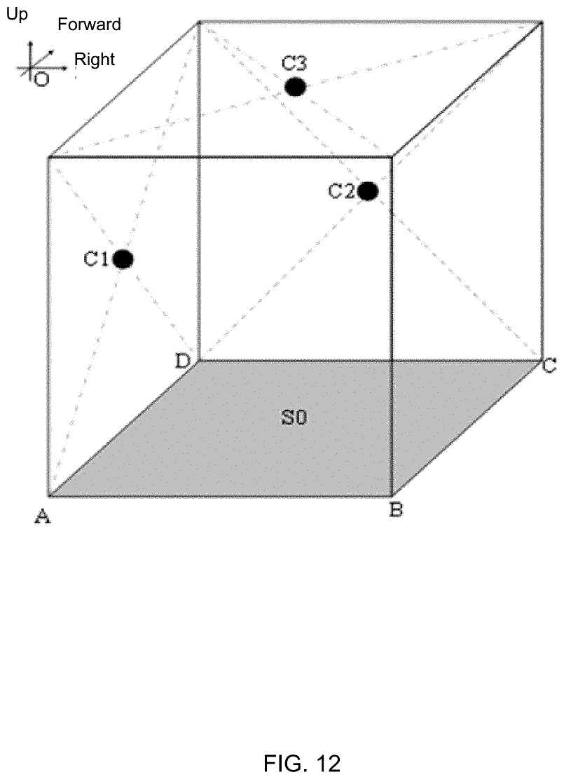

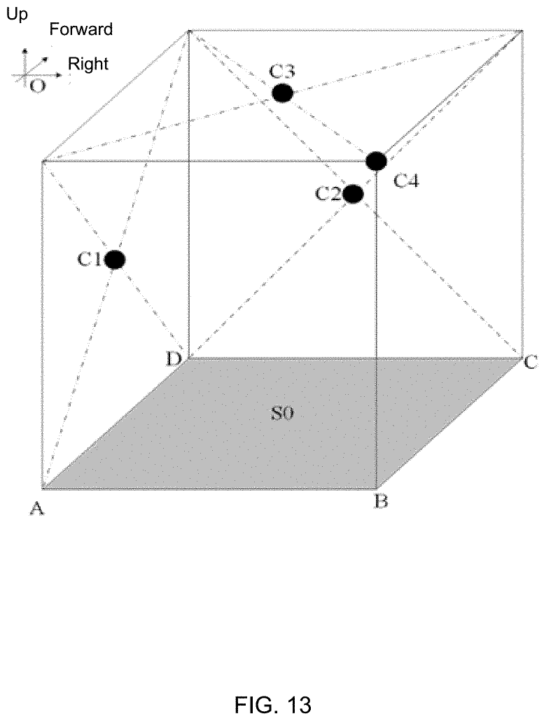

In some embodiments, a plurality of external cameras (or a camera module) may be arranged in a designated fitness place, and the plurality of external cameras (or a camera module) may respectively be arranged in different directions. In some embodiments, a shooting angle of view may be determined according to possible fitness items in the fitness place, the respective arrangement directions of the plurality of external cameras (or a camera module) may be determined according to the shooting angle of view. For example, as shown in FIG. 12, there are three external cameras (or a camera module) C1, C2 and C3 arranged in the fitness place ABCD (i.e., S0), and the three cameras (or a camera module) C1, C2 and C3 are located at the right side (the left side in the figure, or the right side in the figure), right front, and right top the exercise area to capture the side, front, and top views of the user. For another example, as shown in FIG. 13, there are four external cameras (or a camera module) C1, C2, C3 and C4 arranged in the fitness place ABCD (i.e., S0). Different from the arrangement shown in FIG. 12, an external camera (or a camera module) C4 may also be arranged at the right rear corner of the upper right to collect an axonometric view of the user. It may be understood that, based on the examples and descriptions in FIG. 12 and FIG. 13, the number of external cameras (or a camera module) may be increased to more, and the shooting angle and arrangement orientation of each camera (or a camera module) may be determined according to the actual situation.

It should be noted that the fitness place 50 in FIG. 12 and FIG. 13 is not limited to the shape shown in the figure, this is only an example, and is intended to illustrate the arrangement orientation of a plurality of external cameras (or a camera module). Obviously, the fitness place may also be circular, triangular, irregular, etc.

In some embodiments, when the external cameras (or a camera module) include a plurality of cameras, the system for assisted fitness may preset the corresponding relationship between the fitness guidance course and the turning on and turning off each external camera (or a camera module). After the user determines the upcoming fitness guidance course, the system for assisted fitness may determine the external camera (or a camera module) that needs to be turned on according to the fitness guidance course selected by the user. For example, the upcoming fitness guidance course of the user may be push-ups. When the user is doing push-ups, the system for assisted fitness needs to obtain an image on the side of the user to determine whether the action is standard. Therefore, the system for assisted fitness may only turn on the cameras located on the side (right or left). For another example, the upcoming fitness guidance course of the user is jumping jack. When the user is doing jumping jack, the system for assisted fitness needs to obtain the image in front or behind the user to determine whether the user action is standard, so the system for assisted fitness may only turn on the front or rear camera.

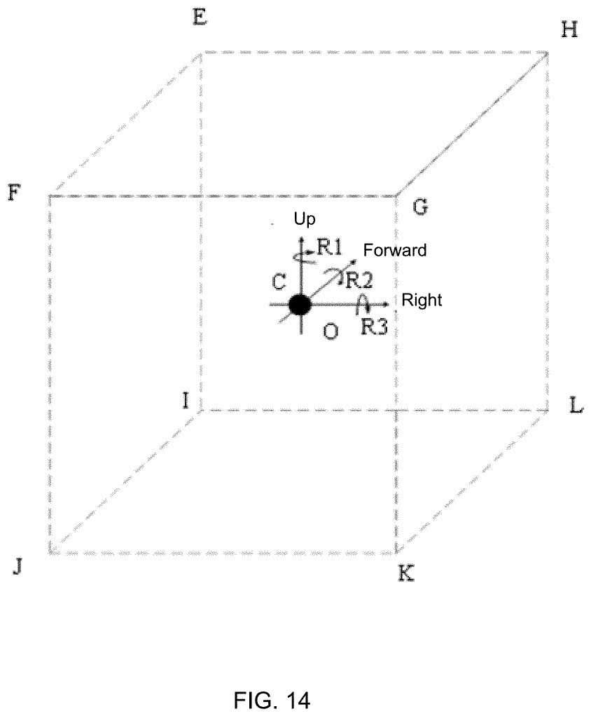

In some embodiments, the external camera (or a camera module) may have a plurality of freedom degrees in space. For example, as shown in FIG. 14, the external camera (or a camera module) C may have 6 freedom degrees (3 freedom degrees for translation in the front-rear, up-down, and left-right directions, as well as the freedom degree R1 for rotation around the up-down axis, the freedom degree R2 for rotation around the front-rear axis, and the freedom degree R3 for rotation around the left-right axis) in space, and the external camera C may move to any position within the range of space EFGHIJK, and orient the lens toward any direction based on the 6 freedom degrees.

In some embodiments, the external camera (or a camera module) may detect the position of the captured human body image within the field of view. When the external camera (or a camera module) detects a human body image but the human body image is not in the center of the field of view, the external camera may adjust its own shooting angle based on the body part (e.g., head, neck, etc.) of the user appearing in the field of view to make the human body image in the field of view as central as possible, and track the human image in the field of view. For example, when the user is jumping jack, there is a change in the horizontal height. At this time, the external camera (or a camera module) located in the right front (or right behind) of the user may be adjusted (e.g., the camera rotates up and down as the user bounces up and down) in the direction of the up and down freedom degrees based on the horizontal height of the user action and the user's head to adapt to the user's fitness action and capture images.

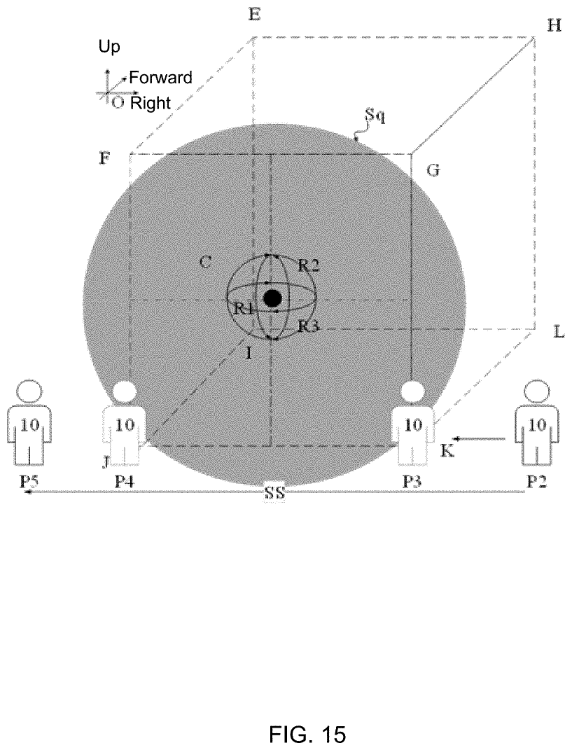

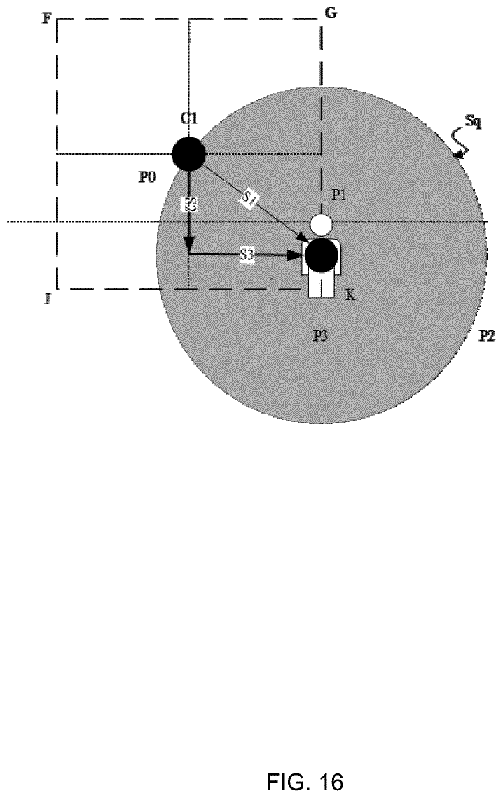

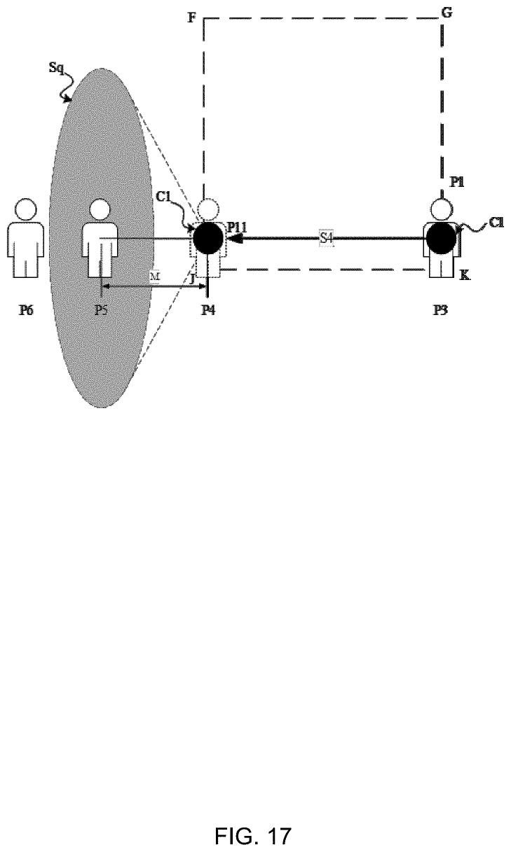

Below illustrates how the external camera (or a camera module) adjusts its own shooting angle of view based on the captured human body image and tracks the portrait in the field of view in conjunction with FIGS. 15 to 17. As shown in FIG. 15, the camera (or a camera module) C may have 6 freedom degrees and may exercise within the range of space EFGHIJK. At this time, the lens of the camera (or a camera module) C is facing right behind, the camera (or a camera module) C is located at the center point P0 of the plane FGKJ, and the person 10 moves from the point P2 to the point P5 along the route SS (the route SS may be parallel to the plane FGKJ). The viewing angle range of the camera (or a camera module) C is Sq. As shown in FIG. 16, when the person 10 moves from the position P2 to the position P3, the camera (or a camera module) C may move from the position P0 to the position P1. The movement distance may be 51, and the movement path may be downward moving distance S2 and right moving distance S3. At this time, the person 10 may be located at the center of the field of view of the camera (or a camera module) C (e.g., C1). As shown in FIG. 17, when the person 10 moves to the point P4, the camera (or a camera module) C (e.g., C1) is at the limit position of the left and right freedom degrees, and may rotate around the axis of the freedom degree R1, and continue to track the person 10. When the person 10 moves to point P5, the maximum focal length range M of the camera (or a camera module) C (e.g., C1) has been reached. At this time, if the person 10 moves to the point P6, the camera (or a camera module) C (e.g., C1) may no longer be able to track the person 10.

In some embodiments, a plurality of external cameras (or a camera module) may be placed within a designated fitness place. When the user escapes from the trackable range of the current camera (or a camera module), the system for assisted fitness may control one or more cameras (or camera modules) adjacent to the current cameras (or camera modules) to continue tracking the user based on the biometric features of the tracked user.

In some embodiments, the camera module that captures exercise images may be a camera module in a third-party apparatus. The third-party apparatus may communicate with the system for assisted fitness. After the camera module in the third-party apparatus collects the exercise image, the third-party apparatus may transmit the collected exercise image to the system for assisted fitness. The third-party apparatus may include, but is not limited to, a combination of one or more of the following: a smart wearable apparatus (e.g., a smart bracelet, a smart watch and/or a smart headband, etc.), a mobile communication apparatus (e.g., a mobile phone and/or a tablet computer, etc.) and/or a desktop apparatus (e.g., a desktop computer, etc.), etc.

The used fitness apparatus may refer to the fitness equipment and/or accessories used by the user in the fitness process. The fitness apparatus used may include, but is not limited to, a combination of one or more of the following: a stand-alone apparatus (e.g., a treadmill, a rowing machine, an exercise bike, an abdominal machine, a trans butterfly machine, etc.), a dumbbell, a barbell, an abs wheel, an elastic ring, etc. The Fitness accessory may include, but is not limited to, a combination of one or more of the following: a yoga mat, a boxing glove, a wristband, a smart cloth, or the like.

In some embodiments, the system for assisted fitness may perform image recognition on the exercise images to identify the fitness apparatus used by the user. In some embodiments, the system for assisted fitness may determine the fitness apparatus used by the user based on whether the data of action is collected by sensors disposed on the fitness accessory or fitness apparatus. In some embodiments, the system for assisted fitness may determine the fitness apparatus that the user may use based on the fitness guidance course used by the user or the fitness requirements of the user. More descriptions of the fitness guidance courses used by the user, reference may be found in the descriptions of the fitness guidance courses in the following paragraphs. More descriptions of the fitness requirements of users, reference may be found in the descriptions of fitness requirements below.

The exercise index parameter may be the data used to represent the corresponding index of various sports items during the user's fitness process. The exercise index parameter may be determined according to different fitness programs. For example, when the fitness item is running, the exercise index parameter may include, but is not limited to, a combination of one or more of the following: pace, distance, incline, or the like. For another example, when the fitness item is push-ups, the exercise index parameter may include, but is not limited to, a combination of one or more of the following: quantity, time, frequency, or the like. For another example, when the fitness program is plank, the exercise index parameter may include time.

The exercise physiological parameter may refer to a physiological parameter of the user during the fitness process of the user. The exercise physiological parameter may include, but is not limited to, a combination of one or more of the following: the user's body temperature, sweat volume, heart rate, respiration rate, oxygen uptake, exercise energy consumption, intensity, or the like.

In some embodiments, the system for assisted fitness may obtain the user's exercise index parameters through sensors on the fitness apparatus. For example, the system for assisted fitness may obtain the user's pace, gradient, etc. during running through the treadmill. For another example, the system for assisted fitness may identify the weight of the dumbbell and the action trajectory of the dumbbell through the posture sensor and the weight sensor on the dumbbell. The system for assisted fitness may evaluate the weight level, action range and action frequency of the user's dumbbell action based on the data obtained by the sensor to obtain the user's exercise index parameters.

In some embodiments, the system for assisted fitness may obtain the exercise physiological parameter of the user through sensors (e.g., IMU (attitude sensors), thermometers, pulse sensors, sweat meters, respiration rate monitors, etc.) in the smart device worn and/or carried by the user. The worn and/or carried smart device may include, but is not limited to, a combination of one or more of the following: a smart wristband, a smart finger ring, a smart watch, a smart headband, a smart cloth, a smart phone, etc.

In some embodiments, the system for assisted fitness may also estimate the energy consumption and intensity of the user's exercise and determine the matching degree between the user's current fitness program and the user's physical state based on the user's exercise physiological parameter or the exercise index parameter when the user is exercising, The matching degree may refer to the degree to which the energy consumption and intensity of the fitness item are adapted to the user's physical state.

The user behavior information may also include other information that may reflect the user's fitness behavior. The mode of obtaining the user behavior information may include, but is not limited to, any one of the foregoing methods and a combination thereof. The system for assisted fitness may also obtain the users' behavior information in other ways, which is not limited here.

The user association information may refer to related information such as the user behavior factors or psychological factors that affect the user's fitness behavior. The user association information may include, but is not limited to, a combination of one or more of the following: a fitness requirement, voice data in fitness, a satisfaction/evaluation of the fitness guidance course, or the like.

] A user's fitness requirement may represent the goal or intention that the user desires to achieve through fitness. The user's fitness requirement may include a general requirement and a special requirement. The general requirement may be the fitness requirement of most users, for example, increasing or decreasing body fat percentage, losing or gaining weight, gaining muscle, etc. The special requirement may be the requirement of a small number of users, such as surgical rehabilitation, postpartum recovery, etc.

In some embodiments, the system for assisted fitness may receive the fitness requirement input by the user. For example, the fitness apparatus may display a plurality of fitness requirements on the display apparatus, thereby guiding the user to make selections to obtain the fitness requirements input by the user.

In some embodiments, the system for assisted fitness may predict the user's fitness requirements based on a combination of one or more of obtained historical exercise image data, historically used fitness guidance course data, historical exercise index parameter data, historical exercise physiological parameter data, and current environmental information. For example, by processing the above data through a machine learning model, a prediction result of fitness requirements may be obtained.

In some embodiments, the system for assisted fitness may also obtain fitness requirements based on various other methods, for example, and fitness requirements may be obtained through semantic analysis on collected voice data of the user when performing fitness, which is not limited here.

The voice data during the fitness may refer to the voice information collected by the system for assisted fitness based on a voice acquisition apparatus (e.g., a microphone) when the user is performing fitness.

In some embodiments, when it is detected that the user is performing fitness (e.g., it is determined that the user enters a fitness state by analyzing the captured exercise images, or the user enters a fitness state by starting to read a subscribed fitness guidance course), the system for assisted fitness may turn on the microphone to collect voice information in the environment in real time, and perform semantic analysis on the voice information to obtain information. For example, the collected real-time voice data of the user includes screams. The system for assisted fitness may determine that the user may encounter danger during the fitness process. At this time, the system for assisted fitness may need to stop fitness-related functions, make an emergency call or call an emergency contact. For another example, the collected real-time voice data of the user may include the sound of rapid breathing. The system for assisted fitness may determine that the current exercise intensity is too high. The user needs to rest and the current fitness item is paused or terminated.

The satisfaction/evaluation of the fitness guidance course may refer to information used to describe the user's feedback on the fitness guidance course. In some embodiments, the user may evaluate the selected fitness guidance course after the user completes the exercise. The system for assisted fitness may use the user's evaluation of the fitness guidance course as a reference factor when adjusting the user's fitness guidance course.

The user association information may also include other information on user behavioral factors and/or psychological factors that may affect the user's fitness behavior. The mode of obtaining user association information may include, but is not limited to, any one of the foregoing methods and combinations thereof. The system for assisted fitness may also obtain user association information in other ways, which is not limited here.

The environment information may refer to the information of the environment where the user is located when the user is exercising based on the system for assisted fitness. The environmental information may include, but is not limited to, a combination of one or more of the following: location information where the user is located, natural environment information, or the like. The place where the user is located may include indoor or outdoor. The location information where the user is located may include, but is not limited to, a combination of one or more of the following: place area, place shape, place ground material, or the like. The natural environment information may refer to the meteorological parameters in the place. The natural environment information may include, but is not limited to, one or more of the temperature, humidity, air pressure (altitude), oxygen concentration, ultraviolet intensity, wind direction, wind speed, etc. in the place.

In some embodiments, the system for assisted fitness may determine place information based on obtained place images. For example, the system for assisted fitness may collect place images through a camera (or a camera module), and analyze the place images to determine the place area, place shape, and place ground material.

In some embodiments, the system for assisted fitness may determine environmental information based on the location of the system for assisted fitness. For example, the system for assisted fitness may query natural environment information from a third-party platform (e.g., a weather network platform) based on the location of the system for assisted fitness. In some embodiments, the system for assisted fitness may determine its location based on its own positioning capabilities. In some embodiments, the system for assisted fitness may determine its location based on the network to which it is connected.

In some embodiments, the system for assisted fitness may also obtain natural environment information through various sensors. The sensors may include, but are not limited to, a combination of one or more of the following: temperature sensors, humidity sensors, barometric pressure sensors, air saturation sensors (oxygen concentration sensors), ultraviolet sensors, anemometers, and other sensors for measuring the natural environment. In some embodiments, various sensors may be built into the system for assisted fitness. In some embodiments, various sensors may also be located in the place where the system for assisted fitness is located, and after various sensors collect the natural environment information in the place, the natural environment information are transmitted to the system for assisted fitness through a communication network.

In some embodiments, the system for assisted fitness may monitor the environmental information in real time, and determine the matching degree of the current environmental information with the fitness guidance course that the user is currently taking. The matching degree may refer to whether each meteorological parameter in the fitness place meets the index requirements of the currently ongoing fitness guidance course for each meteorological parameter. Further, the corrective information may be determined based on the matching degree, for example, the system for assisted fitness may determine whether to adjust the fitness guidance course or adjust the environmental information based on the matching degree. For example, the fitness guidance course is hot yoga. The hot yoga requires an environment of 30° C.-40° C. If the current environment temperature is 29° C., the smart air conditioner may be adjusted through the Internet of Things to increase the environment temperature. More descriptions on corrective information, reference may be found in the related descriptions below.

The mode of obtaining the environmental information may include, but is not limited to, any one of the above methods and combinations thereof. For example, when the location of the system for assisted fitness may not be obtained, the system for assisted fitness may collect various natural environment information in the fitness place through various built-in sensors. The system for assisted fitness may also obtain environmental information in other ways, which is not limited here.

More descriptions of the fitness guidance course of system for assisted fitness, reference may be found in the following descriptions.

The system for assisted fitness may provide the user with a fitness guidance course to guide the user in fitness. In some embodiments, the fitness guidance course may be a pre-recorded fitness-related course, or a course automatically generated according to fitness-related information. In some embodiments, the fitness guidance course may also be referred to as the fitness course. In some embodiments, the system for assisted fitness may also provide users with the fitness guidance course in other ways, for example, the fitness guidance course suitable for users may be generated by synthesizing fitness video clips recommended by the network, which is not limited in the present disclosure.

As previously mentioned, the fitness guidance courses may be pre-recorded courses. Accordingly, the system for assisted fitness may recommend pre-recorded fitness guidance courses for the user. In some embodiments, the system for assisted fitness may display various fitness contents (i.e., the fitness guidance course) on the display interface for the user to select. For example, the user may directly select fitness content that suits them on the display interface according to their own needs, and the system for assisted fitness may directly determine the fitness content selected by the user as the final fitness guidance course.

In some embodiments, the system for assisted fitness may recommend the fitness guidance course to the user without obtaining the user information and fitness-related information. For example, when facing unfamiliar users without the obtained user information, the system for assisted fitness may also implement recommendation of the fitness guidance course. In some embodiments, the system for assisted fitness may recommend fitness guidance courses for the user based on at least one of the fitness guidance course, the fitness coach, or other hot information. For example, the system for assisted fitness may determine the recommended content according to the popularity of the fitness guidance course, and the higher the popularity of the fitness guidance course is, the greater possibility of recommending fitness guidance courses is. For another example, the system for assisted fitness may extract keywords according to recent hot topics to recommend corresponding fitness guidance courses to the user. For example, as summer approaches, the vest line has become a hot topic of discussion. Then the system for assisted fitness may recommend related fitness guidance courses containing the keyword “vest line” to the user. For another example, the system for assisted fitness may determine recommended fitness guidance courses from the course library based on dimensions such as the coach's popularity, and recommend fitness guidance courses of popular coaches to the user.

In some embodiments, the system for assisted fitness may perform personalized customized recommendations for the user based on the obtained information (e.g., user information, environmental information), thereby providing the user with more and more suitable course selections. The more descriptions of obtaining information, reference may be found in the related descriptions of the obtaining information section, which may not be repeated here.

In some embodiments, the system for assisted fitness may obtain the environment information where the user is located, and perform course recommendation according to the obtained environment information for the user to choose. More descriptions of the obtaining environmental information, reference may be found in the related descriptions in the section about obtaining information. For example, the system for assisted fitness may preset at least one corresponding environmental trigger condition for each fitness guidance course. When the obtained environmental information satisfies a certain environmental trigger condition, at least one or more fitness guidance courses corresponding to the environmental trigger condition may be recommended. For example, the system for assisted fitness may pre-set that the environmental trigger conditions for courses such as jogging, yoga, etc. may be the altitude being less than 500 m and the temperature being in the range of 10° C.-25° C. When the obtained current environmental information of the user is: in a certain urban area, an altitude of 450 m and a temperature of 15° C., then the system for assisted fitness may recommend fitness guidance course such as jogging, yoga, etc. corresponding to the environmental trigger conditions satisfied by the environmental information for the user.

In some embodiments, the system for assisted fitness may perform course recommendation according to the obtained specific information of the place where the user is located. The specific information of the place may include information such as place area, ground material, ground smoothness, ground softness and hardness. etc. For example, the system for assisted fitness may recommend corresponding fitness guidance courses to the user according to the obtained floor area information of the place where the user is located. For example, the system for assisted fitness may preset a floor area required for a hula hoop course to be at least 3 square meters (m²). When it is obtained that the user's environment is larger than 3 m², the hula hoop course may be recommended. For another example, the system for assisted fitness may recommend a corresponding fitness guidance course according to the obtained ground material of the place, the smoothness of the ground, or the degree of softness and hardness of the ground. The system for assisted fitness may set the standard that the floor needs to reach for some special exercise courses. When the obtained ground information of the place reaches the corresponding standard, the system for assisted fitness may recommend the corresponding fitness guidance course to the user. For example, the system for assisted fitness may preset performing a yoga course in the place with a relatively smooth and flat ground. When it is determined that the ground of the place where the user is located reaches the corresponding standard, a yoga course may be recommended.

In some embodiments, when the system for assisted fitness recommends a course in combination with the place where the user is located, it may also recommend and display the best exercise position suitable for the currently recommended course in the exercise place for the currently recommended course at the same time. For example, the system for assisted fitness may predict the possible action direction of the user (e.g., predicting through model) in combination with the specific content of the recommended courses. Further combined with the camera layout of the user obtained by the system, a location in the place that is convenient for collecting image information and not affect the user's movement is recommended to the user. For example, the system for assisted fitness may determine a suitable activity area based on the layout of the camera, and then use the position overlapping with the action direction in the suitable activity area as the optimal action position. The suitable activity area is area composed with the location where the camera may capture the user's whole body as a center, and the default value as the radius.

In some embodiments, the system for assisted fitness may obtain user information and recommend a fitness guidance course to the user based on the user information. More descriptions of the obtaining of user information, reference may be found in the section about obtaining information. In some embodiments, the system for assisted fitness may recommend an initial fitness guidance course to the user based on at least one of personally identifiable information, basic physiological parameters, fitness requirements, satisfaction/evaluation of the fitness guidance course, or the like. In some embodiments, the system for assisted fitness may update or adjust the recommended fitness guidance course based on the user behavior information.

In some embodiments, the system for assisted fitness may recommend the fitness guidance course for the user based on a fitness requirement. For example, after the user directly inputs the items to be performed on the fitness apparatus, the fitness apparatus may recommend the fitness guidance course of the same type of items. For example, if the user inputs running, the corresponding recommendations may include different types of running fitness guidance courses such as long-distance running and sprinting.

In some embodiments, the system for assisted fitness may recommend the fitness guidance course for the user based on the basic physiological parameters of the user. For example, if the user has high blood pressure, the recommended course should not include strenuous exercise. The recommended course may be jogging, etc.

In some embodiments, the system for assisted fitness may recommend a fitness guidance course for the user based on the user's posture data. For example, if the system for assisted fitness obtains that the user is in a sitting state, it may recommend the courses including sitting postures, such as yoga. In some embodiments, the user's gesture data may include a set of key points corresponding to the user actions. For example, a set of action key points such as arms and legs. In some embodiments, the user's posture data may be determined in various ways. For example, it may be determined by identifying the exercise images in the user behavior information.

In some embodiments, the system for assisted fitness may recommend a fitness guidance course for the user based on the fitness apparatus the user uses or is likely to use. For example, if the system for assisted fitness recognizes that the user is holding a dumbbell, it may recommend the fitness guidance course related to the dumbbell. In some embodiments, the system for assisted fitness may determine the fitness apparatus that the user is likely to use in various ways. For example, the system for assisted fitness may determine the fitness apparatus around the user as fitness apparatus that user is likely to use through the image recognition. For another example, the fitness apparatus used in the fitness guidance course that is highly praised by users in historical is determined as the fitness apparatus that user is likely to use.

In some embodiments, the system for assisted fitness may recommend a fitness guidance course for the user based on the historical data of the user's exercise. For example, the system for assisted fitness may obtain the historical data of the user's exercise, and make recommendations according to the fitness guidance course in the historical data of the user's exercise. When making specific recommendations, the user's fitness preferences or requirements as well as the user's physical level, etc. may be predicted based on the fitness guidance course in the history data of the user's exercise, and fitness guidance course that may be more inclined in their current state is recommended to users based on the information. For example, the system for assisted fitness may recommend a course corresponding to the same physical level to the user based on the physical level corresponding to the fitness guidance course in the history data of the user's exercise. For another example, the system for assisted fitness may extract the teaching features of the coach that the user has followed, perform cluster analysis on the teaching features of different coaches, analyze the type of coaches that the user likes, and recommend the fitness guidance course of the coaches corresponding to the type of teaching features to users.

In some embodiments, the system for assisted fitness may recommend a fitness guidance course for the user based on the user's personally identifiable information. For example, the system for assisted fitness may use the model to predict the part of the user's body that are prone to strain at work according to the occupational information in the user's personally identifiable information, and recommend the fitness guidance course that is conducive to stretching or repairing the part.

In some embodiments, the system for assisted fitness may recommend a course for the user based on other users similar to the user. Other users similar to the user may refer to other users whose personally identifiable information (e.g., occupation, gender, age, etc.) and basic physiological parameters (e.g., height, weight, blood sugar, blood pressure, blood lipids, etc.) are similar to the current user. For example, the system for assisted fitness may recommend fitness guidance courses that are highly praised by other users similar to the user to the user. In some embodiments, the system for assisted fitness may determine similar other users based on user information (e.g., personally identifiable information, basic physiological parameters, etc.).

In some embodiments, the system for assisted fitness may recommend a fitness guidance course for a user based on various combinations of personally identifiable information, the basic physiological parameter, the fitness requirement, the satisfaction/evaluation of the fitness guidance course, historical data, or fitness apparatus. For example, the fitness guidance courses that match their fitness requirements and exercise to their strained parts, as well as their physical state are recommended to users.

In some embodiments, since the user's situation may change with time, when combining the obtained user information, the system for assisted fitness may also set the credibility weight for the data in combination with the time interval between the time when the user information is generated and the current time. The data is close to the current data, indicating that the credibility is high. Accordingly, the system for assisted fitness may determine the recommended fitness guidance course according to the user information with high credibility.

In some embodiments, the system for assisted fitness may update the recommended fitness guidance course based on user information during the fitness. More descriptions may be found in the related content of adaptively adjusting of the course selected by the user in combination with the obtained user status data in the section on corrective information below.

In some embodiments, the system for assisted fitness may recommend a corresponding course to the user according to the obtained number of current users. As previously mentioned, a plurality of users may perform fitness through one or more fitness apparatus in the system for assisted fitness. A plurality of users may perform fitness interactively or independently. In some embodiments, the system for assisted fitness may recommend a course based on the number of users, the number of fitness apparatus, and/or whether a plurality of users perform fitness interactively. For example, if the system for assisted fitness obtains that the number of users currently using the system is one person, it may recommend fitness guidance courses that may be performed by one person, such as running, yoga, etc. to the user. For another example, if the system for assisted fitness obtains that the number of users currently using the system is multiple and there is one fitness apparatus, it may recommend fitness guidance courses that may be performed by the corresponding number of users according to the corresponding number of users. For example, when the system for assisted fitness obtains that the number of users currently using the same apparatus is two, and the number of fitness apparatus is one, it may recommend pair dance, two-person yoga, etc. For another example, if the system for assisted fitness obtains that the number of users currently using the system is multiple, and the plurality of users perform fitness independently, a fitness guidance course that may be performed by a single person may be recommended for each user. For another example, if the system for assisted fitness obtains that the number of users currently using the system is multiple, and there is a plurality of fitness apparatus, the plurality of fitness apparatus may be remotely connected, and the number of users currently connected is obtained, and course may be recommended based on the number of users currently connected. For example, it may recommend fitness guidance courses suitable for the current number of users currently connected. For example, pair dance and two-person yoga may be recommended for two people, and speed running may be recommended for more than two people.

In some embodiments, when recommending a single-person fitness guidance course to a single user, targeted recommendations may be made based on user information of each user. In some embodiments, when recommending interactive fitness for the plurality of users, the system for assisted fitness may recommend courses that may meet the fitness conditions of all interactive users based on the number of users participating in interactive fitness, the situation of each user's exercise place, etc. The fitness conditions may include place area conditions, user basic physiological parameter conditions, or the like. For example, if all the users participating in the interactive fitness have an area of an exercise place that meets the fitness conditions for aerobics, the system for assisted fitness may recommend the fitness guidance course related to the group aerobics to the users. For another example, if the basic physiological parameters of all users participating in the interactive fitness meet the load corresponding to the aerobics exercise, the system for assisted fitness may recommend the fitness guidance course related to the group aerobics to the users.

In some embodiments, the system for assisted fitness may also recommend a fitness guidance course to users in other ways. For example, the related information may be processed through the preset recommendation model, and the recommended fitness guidance course may be obtained, which is not limited in the present disclosure.

As previously mentioned, the fitness guidance course may be automatically generated. In some embodiments, the system for assisted fitness may automatically generate a fitness guidance course for the user based on the obtained information. More descriptions of obtaining information, reference may be found in the corresponding content in the section about obtaining information.

In some embodiments, the system for assisted fitness may generate a fitness guidance course based on non-process information. The non-process information may include environmental information, user description information, fitness requirements, or the like. For example, an aerobic exercise course may be generated for a user in a high-altitude environment to prevent the user from developing altitude sickness and ensure the user's personal safety during the fitness process.

In some embodiments, the system for assisted fitness may utilize the coaching teaching content to generate a fitness guidance course. For example, the system for assisted fitness may obtain a large amount of coaching teaching content, use a coaching action as a standard action, and extract the key points of each standard action to obtain a standard action library. Then the system for assisted fitness may select one or more standard actions based on environmental information, fitness requirements and/or user description type information. Then, according to the key points corresponding to each standard action in the standard action library, the system for assisted fitness may generate a corresponding fitness guidance course using a virtual character to implement the corresponding standard actions according to the key points. In some embodiments, a user's avatar or other user's favorite avatar, etc. may also be placed on the virtual character by adopting a technology similar to face swapping, so that the user has a stronger sense of substitution and the user's fitness efficiency is improved. More descriptions of the standard action, reference may be found in related descriptions of the corrective information section.

In some embodiments, the system for assisted fitness may also generate the fitness guidance course in other ways. For example, the coach's standard action may be decomposed into a plurality of unit actions and stored in the database, and then the obtained user-related information may be processed through the machine learning model to generate the material tags, and the corresponding unit actions may be obtained from the database according to the material tags, and then the obtained unit actions may be processed according to the rules to generate a fitness guidance course.

In some embodiments, the system for assisted fitness may generate a personalized fitness guidance course based on the obtained process information. The process information may include user behavior information or historical data of the user. For example, the system for assisted fitness may integrate the historical fitness guidance courses to generate fitness guidance courses according to the items that the user has exercised before. For another example, the system for assisted fitness may obtain the image information of the user during fitness process, determine the historical fitness guidance courses that the user may have participated in according to the image information, and then extract the standard actions corresponding to the historical fitness guidance courses from the standard action library to generate the fitness guidance courses. In some embodiments, the system for assisted fitness may obtain the user's exercise data (e.g., historical sweat volume, heartbeat, breathing rate, etc.) during fitness process, determine the exercise intensity that the user may adapt to according to the exercise data, select the standard action corresponding to the exercise intensity from the standard action library, and generate the corresponding fitness guidance courses by integrating the standard action corresponding to the exercise intensity.

In some embodiments, when the system for assisted fitness does not obtain the user's process information initially, the system for assisted fitness may first generate a fitness guidance course based on the obtained non-process information, and then combine the obtained process information to generate or adjust the subsequent fitness guidance course.

In some embodiments, the system for assisted fitness may generate the corresponding fitness guidance course according to the current number of users, the fitness apparatus that each user uses or may use, the exercise place where the user is located, etc. in combination with the physical status of each user. For example, under a single system, the system for assisted fitness may generate a fitness guidance course that corresponds to the number of users and meets the user's current used fitness apparatus and physical status according to the used fitness apparatus, fitness status, etc. of each among the one or more users. For example, when the place of the current user is large enough and the physical state of the user is relatively good, but the user does not have fitness apparatus, aerobics corresponding to the number of users may be generated. For another example, under a plurality of systems, the system for assisted fitness may obtain the number of users currently online and the fitness places, fitness apparatus, and physical status of each user, and the generate fitness guidance courses corresponding to the number of online users, fitness apparatus, and physical status. For example, when some online users have limited space and/or limited apparatus, the system for assisted fitness may generate fitness content with less restrictions on the space, such as speed racing.

In some embodiments, the system for assisted fitness may generate a fitness guidance course using a course generation model based on the obtained information. In some embodiments, the course generation model may a machine learning model, for example, a deep neural network (DNN), a convolutional neural network (CNN), or the like.

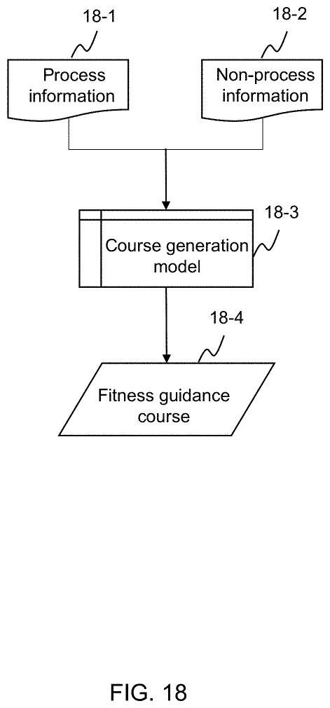

FIG. 18 is a schematic diagram of generate fitness guidance courses using the course generation model according to some embodiments of the present disclosure. As shown in FIG. 18, in some embodiments, the input of a course generation model 18-3 may be obtained information, such as process information 18-1 and non-process information 18-2, and the output of the course generation model 18-3 may be the corresponding fitness guidance course 18-4. For example, in the use of the user, after obtaining the non-process information and the process information, the obtained information may be input into the pre-trained course generation model 18-3, and the course generation model 18-3 may output the matching course (the specific implementation process may refer to existing poetry, music, video creation AI, etc.)

In some embodiments, the course generation model may be trained with a plurality of labeled training samples. For example, the system for assisted fitness may obtain a large number of non-process sample information and process sample information with fitness guidance course labels in advance, input non-process sample information and process sample information into the initial course generation model, and construct a loss function based on the labels and the output results of the initial course generation model, iteratively update the parameters of the initial course generation model through gradient descent or other methods based on the loss function. When the preset conditions are met, the model training is completed, and the trained course generation model is obtained. The preset conditions may be that the loss function converges, the number of iterations reaches a threshold, or the like.

In some embodiments, the system for assisted fitness may also generate the fitness guidance course based on other methods, which is not limited in the present disclosure.

In some embodiments, the fitness apparatus may display fitness-related content, such as fitness guidance courses, fitness data, etc., through the display apparatus. In some embodiments, the display apparatus may include one or a combination of one or more of a display, a mirror glass, and an apparatus using holographic projection, air projection and other related 3D technologies. The display may include a display in the form of projection type, stereo imaging type, light-emitting diode, etc., and the mirror glass is mirror glass with a certain ratio of reflection and perspective.

As previously mentioned, the display apparatus of the fitness apparatus may include a display and a mirror glass. In some embodiments, the display may be used to display fitness-related content, and the mirror glass may be used for reflection to facilitate the user to see the action posture during the exercise. In some embodiments, the display and the mirror glass may be combined to display the user's fitness item. For example, the display may be used to show the actions on one side of the user, and the mirror glass may allow the user to see the actions on the other side of the user through the mirror.

In some embodiments, the display apparatus of the fitness apparatus may include at least one display. The fitness apparatus may be displayed through the display. For example, the fitness apparatus may display the user action and the coaching action through the display.

In some embodiments, the fitness apparatus may display all content on one display to allow the user to view all content on one display.

In some embodiments, the fitness apparatus may perform fusion display on two or more videos. For example, the fitness apparatus may perform fusion display on the coaching action video (i.e., the fitness guidance course) and the user action video. The fusion display may refer to the simultaneous display of coaching action video and user action video on the same display interface.

In some embodiments, display effects of fusion display of the coaching action video and the user action video may be various. For example, the display interface may be partitioned on the same display to display the coaching action video and the user action video respectively. For another example, the image of the coach and the image of the user may be overlapped and displayed in the same display area of the same display, so that the user may better refer to the coaching actions to exercise.

In some embodiments, the fitness apparatus may perform fusion display on the coaching action video and the user action video in various ways. For example, the system for assisted fitness may generate user space timing information by extracting the related information of the user action video stream and the coaching action video stream, and fuse the coaching action video with the user action video according to the user space timing information, and then the fitness apparatus may display the fused result on a display. The timing information may refer to the chronological ordering between two or more objects.

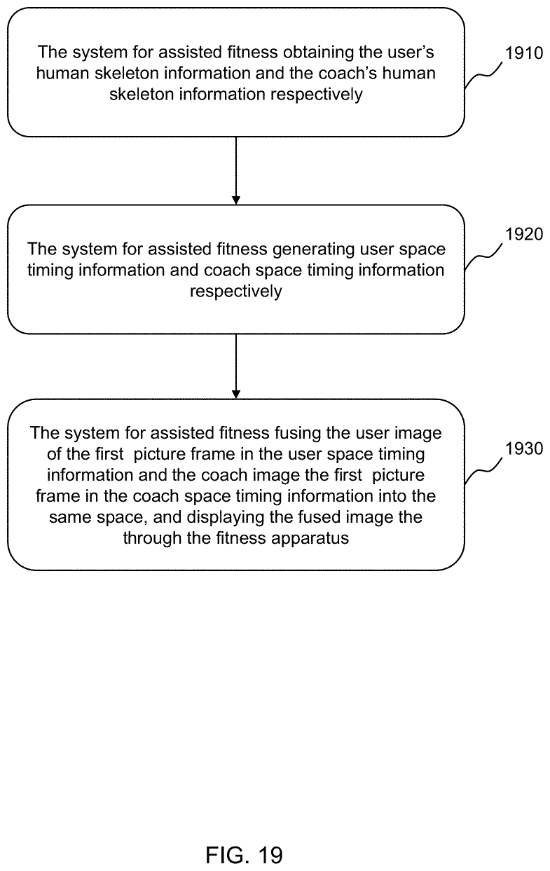

For example, the system for assisted fitness may obtain the key node positions of the human skeleton of the user and the coach according to the user action video stream and the coaching action video stream to generate the user's human skeleton information and the coach's human skeleton information, and generate a fusion video based on the user's human skeleton information and the coach's human skeleton information for fusion display. In some embodiments, the method for fusion display based on the user's human skeleton information and the coach's human skeleton information may be implemented as shown in FIG. 19. As shown in FIG. 19, the specific implementation operations of the method are as following operations.

In operation 1910, the system for assisted fitness obtaining the user's human skeleton information and the coach's human skeleton information respectively.

In some embodiments, the system for assisted fitness may obtain the user action video stream through the camera, and process each frame of pictures to generate the user's human skeleton information (e.g., the user's skeleton information may be obtained by obtaining the main key node positions of the human skeleton). In the same way, the coach's video is processed in the same way to obtain the coach's human skeleton information.

In operation 1920, the system for assisted fitness generating user space timing information and coach space timing information respectively.

In some embodiments, the timing in the space timing information may refer to the number of frames of the video, and the space may refer to the corresponding three-dimensional coordinate information of the human body in each frame of pictures.

In some embodiments, the system for assisted fitness may create three-dimensional coordinates of the user's human body according to the user's location and the user's human skeleton information, and then generate user space timing information according to the three-dimensional coordinates of the user's human body and the number of frames of pictures of the user action video stream. In the same way, the system for assisted fitness may generate the coach space timing information in the same way.

For example, after creating the three-dimensional coordinates of the user's human body by extending the plane XY with the user's yoga mat, taking the vertical direction of the user's body as the Z-axis, then each frame of pictures of the user action video has corresponding three-dimensional coordinates of the human body, and all the frame of pictures of a single video and the corresponding three-dimensional coordinates of the human body form the space timing information.

In some embodiments, for the corresponding coach space timing information of the same coaching action video, the system for assisted fitness may only perform generation operation based on the coaching action video, and save the generated coach space timing information, which may be called directly for subsequent video fusion.

In operation 1930, the system for assisted fitness fusing the user image of the first picture frame in the user space timing information and the coach image of the first picture frame in the coach space timing information into the same space, and displaying the fused image through the fitness apparatus.

Each frame of pictures in the space timing information corresponds to three-dimensional coordinates of the human body, and the coordinates of the human body images corresponding to the three-dimensional coordinates of the human body may be unified. In some embodiments, the system for assisted fitness may fuse the user image of the first picture frame and the coach image of the first picture frame into the same space, and then fuse the user image of the second picture frame and the coach image of the second picture frame into the same space, until the user image and coach image of all picture frames in the video are continuously fused into the same space in sequence, the fusion of the two pictures may be processed by multiple frames to form the corresponding fusion video. In some embodiments, after the fusion video is formed, the system for assisted fitness may display the fusion video through the fitness apparatus corresponding to the user/coach.

In some embodiments, the coaching action video may be obtained from a fitness guidance course, and the video of the user action may be obtained through a camera. More descriptions of the camera, reference may be found in the foregoing descriptions, which may not be repeated here.

In some embodiments, the system for assisted fitness may also use other methods to perform fusion display on the user action video and the coaching action video, which may not be limited in the present disclosure.

In some embodiments, the fitness apparatus may display two or more videos on a split screen. In some embodiments, the fitness apparatus may display the coaching action video and the user action video on a split screen. The display on a split screen refers to two or more images are displayed on different areas of the screen. The fitness apparatus may display coaching action video and user action video on a split screen in various ways. For example, the fitness apparatus may superimpose one display window with another display window on one display window, which are respectively used to display the coaching action video and the user action video. For another example, the fitness apparatus may place two display windows side by side, which are respectively used to display the coaching action video and the user action video.

In some embodiments, the fitness apparatus may display different content on different display apparatus to allow a user to view different content on different display apparatus.

In some embodiments, the fitness apparatus may include a plurality of display apparatus. The plurality of display apparatus may be used to display the coaching action video and the user action video, respectively.

In some embodiments, in order to meet the diverse requirements of users, there may be various display modes of the fitness apparatus. For example, if the display effect required by the user is that the size of the human skeleton of the user's image and the coach's image is the same, or one of the human skeleton of the user's image and the coach's image is larger, the fitness apparatus may proportionally enlarge or reduce the human skeleton of the user or the coach for display. For another example, if the display effect required by the user is that the backgrounds of the user's image and the coach's image are consistent, the fitness apparatus may perform image segmentation processing on each frame of the coaching action video and the user action video through binarization, clustering, histogram and other image segmentation techniques, and replace non-human images of coaching action video and user action videos with a unified background For another example, if the user does not want to see himself/herself in the video, the fitness apparatus may display a 3D model of the human body instead of the user. More descriptions of obtaining user information may be found in the section about obtaining information, which may not be repeated here. At the same time, it should be noted that there may be various methods for generating a 3D model of a user's human body, which may not be limited in the present disclosure.

In some embodiments, the display apparatus of the fitness apparatus may include apparatus related to 3D technology such as holographic projection, air projection, etc. In some embodiments, the fitness apparatus may display all content on one screen in various ways to allow the user to view all content on one screen. For example, the system for assisted fitness may obtain a new video by fusing the coaching action video and the user action video, and then use the corresponding 3D video production technology to make the new video into a holographic projection video, and perform holographic projection through the fitness apparatus. For another example, the system for assisted fitness may first make the user image and the coach image into an overlapping display image, and then make the overlapping display image into a holographic projection video, and perform holographic projection through the fitness apparatus. In some embodiments, the fitness apparatus may display different content on different projection screens in various ways to allow a user to view different content on different projection screens. In some embodiments, the fitness apparatus may be provided with a plurality of projection screens for displaying the holographic projection images of the coaching action video and the user action video, respectively.

] In some embodiments, there may be various solutions for displaying the coaching action video and the user action video through holographic projection. For example, the holographic projection image of the coach may be made in advance, and the holographic projection image of the user may be made in real time, and the holographic image of the user and the holographic image of the coach may be displayed on two parts of the screen respectively.

In some embodiments, the system for assisted fitness may collect user information and environmental information from a plurality of directions to obtain the audio and video information of the user. In some embodiments, the system for assisted fitness may process the collected user information to generate a holographic image of the user. In some embodiments, before processing the collected information, the user video information of each direction may be extracted and segmented to obtain a frame picture of the user. In some embodiments, the processing of the collected information may include preliminary processing of user information, establishment of a splicing mapping relationship, frame picture splicing, or other operations.

In some embodiments, the system for assisted fitness may first extract the user from the complex environment based on the collected user information, and complete the preliminary processing of the user information in the real mode of the holographic display. In some embodiments, the system for assisted fitness may establish a splicing channel, and establish a splicing mapping relationship for matching the user information in all directions with the splicing channel to realize the data basis for the reproduction of holographic real images. In some embodiments, the system for assisted fitness may splice a frame picture of the user in all directions to form a holographic image of the user based on the FBO technology. The FBO technology may not be repeated here.

In some embodiments, the system for assisted fitness may locate the user's eyes in the user information through the human eye recognition module, etc. At the same time, the projection screen or the projection apparatus may be designed as a movable structure to control the projection screen or the projection apparatus to move with the positioning of the human eye, and maintain the mirror position of the human eye and the projection apparatus relative to the projection screen. In this way, the projection screen or the projection apparatus may be moved with the user, avoiding the problem of poor course display due to the user's exercise state during the fitness process, ensuring that the user may receive a clear 3D image when exercising, and improving the user experience.

In some embodiments, if a 3D display effect with a larger viewing angle is to be obtained, a projection method on a 3-dimensional medium may also be used. For example, a display apparatus composed of a stereoscopic display module and a stereoscopic scattering screen may be used, and the stereoscopic scattering screen scatters the light projected by the stereoscopic display module to achieve a 3D effect. The method for realizing 3D imaging by light scattering may not be repeated here.

In some embodiments, the fitness apparatus may be used in a mix of display modes. For example, the fitness apparatus may perform mixed display such as monitor display, projection display, and 3D display according to the actual situation. For another example, the fitness apparatus may combine a plurality of display modes for joint display when the number of users to be displayed is large or the users have different display requirements.

In some embodiments, the fitness apparatus may also implement various display requirements of the user in other ways, which are not limited in the present disclosure.

The fitness apparatus may display the coaching actions to guide the user to exercise according to the coaching actions. The coach may be a real people, a robot, etc. The aforementioned fitness guidance course may include the coaching actions. As mentioned above, the fitness guidance course (including the coaching actions) may be pre-recorded or automatically generated.

In some embodiments, the fitness guidance course may be adjusted according to the situation and updated to a course that better meets the requirements. More descriptions may be found in the related descriptions of the corrective information section below. In some embodiments, the user's body, occupation, age, etc. are be different, or the user's fitness scene is different, causing that the different users have different requirements for the fitness guidance course. The system for assisted fitness may match different requirements by controlling the display or playback of the fitness guidance course. The system for assisted fitness may adjust the playback of the pre-recorded content according to the user's information. For example, the playback speed of pre-recorded content may be adjusted, and the frequency of the displayed coaching action may be affected by changing the playback speed. For another example, the image scale, resolution, etc. in the pre-recorded content may be adjusted, and the displayed coaching action range may be affected by changing the image scale. The system for assisted fitness may add new content to the pre-recorded content for display according to requirements. For example, the system for assisted fitness may recognize the user's appearance (e.g., looks and/or wearing, etc.) and/or scene, etc., and replace the coach's scene and/or appearance in the pre-recorded content with the user's scene and/or appearance to make the user's experience more realistic. For another example, the system for assisted fitness may add animations such as emotions (e.g., sweat, expressions, etc.) to the pre-recorded content according to the time length and amplitude of the coaching actions. For another example, the system for assisted fitness may add or replace fitness apparatus etc., in the pre-recorded content. The adjustment of the fitness guidance course by the system for assisted fitness may include, but is not be limited to any one of the above methods and their combination.

In some embodiments, the system for assisted fitness (e.g., a fitness apparatus) may broadcast a coaching action or the fitness course for the user.

In some embodiments, the fitness apparatus may provide the user with the coaching actions through a combination of live and pre-recorded. For example, when the fitness apparatus determines that the coaching action is a preset action, it may automatically switch to a pre-recorded video containing the preset action. The preset action may be customized according to user requirement. For example, the preset action may be an action that may not be displayed in the live broadcast or a detailed action.

In some embodiments, the fitness apparatus may monitor and display the actual action of the user. For example, the user action may be photographed by the camera and displayed accordingly. More descriptions of the mode of displaying, reference may be found in the above-mentioned display section. More descriptions of the camera, reference may be found in the previous section.

In some embodiments, the fitness apparatus may display the standard action for the user to guide the user to perform the standardized fitness action. The standard action represents the standard that the user should meet when operating the action. The standard action may be pre-generated, for example, the standard action may be generated based on the coaching actions. More descriptions of standard actions, reference may be found in the related descriptions in the corrective information section.

In some embodiments, during the fitness process of the user, the fitness apparatus may display to the user the difference between the standard action and the user's actual action. The difference between the standard action and the actual action may be obtained by identifying the images of the actual action, for example, identifying the key point position of the actual action in the image, and determining the distance between the key point position of the actual action and the key point position of the standard action, etc. The difference between standard action and actual action may be used as a source or adjustment basis for the standard action, the fitness plan, the pre-recorded content, the follow-up corrective information, or the like. For example, when the actual action is far from the standard action, the system may reduce the difficulty of the standard action (e.g., reducing the action range, etc.).

In some embodiments, the fitness apparatus may display assisted content. In some embodiments, the assisted content may be used to prompt the user or facilitate the user to perform a shortcut operation. For example, the specific display area may be set on the screen for displaying text, icons, etc., so as to provide quick prompts to the user or to perform quick operations by the user. In some embodiments, the fitness apparatus may use various display methods such as item bar display, display of jumping from a dotted box to a solid box, etc. to prompt the user for the completion of the action. For example, the fitness apparatus may display a dotted frame on the display screen in combination with the user's image to give the user action range as a reference. The dotted frame of action range is not same for all users, which may be adjusted in real time according to the non-process information and process information in combination with the user's own situation and the training process. The position of the dotted frame of the action range may represent the position that the standard action needs to reach, and the determination of the standard action may refer to the description of the standard action below.

In some embodiments, the fitness apparatus may individually display one or more of coaching actions, user actions, differential actions, etc. For example, the fitness apparatus may use the aforementioned one display apparatus (including a display screen, a projection screen, etc.) to respectively display one or more of the coaching actions, user actions, differential actions, etc. through a split screen. For another example, the fitness apparatus may use the aforementioned display apparatus to individually display the content that needs to be displayed.

In some embodiments, the fitness apparatus may overlapping display the coaching action and the user action. The fitness apparatus may perform analysis and processing based on the overlapping displayed actions. For example, the fitness apparatus may overlapping display the coaching action and the user action, and determine the difference between the two actions as the action that the user needs to correct. For another example, the fitness apparatus may overlapping display the coaching action and the user action, analyze the standard action corresponding to the user action separately, and set a dotted frame or shadow at the corresponding position in the overlapping screen to indicate the corrective action. For another example, the fitness apparatus may overlapping display the coaching actions and the user actions, analyze the standard action corresponding to the user action separately. In the display apparatus, the display area that needs to be displayed for the user action that needs to be corrected is separately distinguished through split-screen or screen-on-screen, etc.

The display content and display method of the fitness apparatus may include, but are not limited to any one of the above methods and their combination, and the display content may also be displayed in other ways, which may not be limited here.

More descriptions of the corrective information, reference may be found in following descriptions.

In some embodiments, the system for assisted fitness may correct the recorded fitness guidance course that has been generated or selected by the user based on the obtained information to obtain the course that is more suitable for the user.

In some embodiments, the system for assisted fitness may make adjustment and correction on the generated or recommended courses in time based on the obtained process information (more descriptions of the process information, reference may be found in the related descriptions in the fitness guidance course section above). For example, the system for assisted fitness may first recommend a dumbbell-related course to the user according to the obtained information such as the user's place and apparatus. Then, in combination with the obtained exercise data of the user, the system for assisted fitness may judge that the user is struggling to perform the current dumbbell course, correct the recommended course, and re-recommend a more soothing course such as yoga for the user. For another example, when the user has a non-standard exercise action, the system for assisted fitness may automatically repeat the aforementioned non-standard exercise action in a subsequently generated course to meet the user's exercise requirements. The specific implementation method may obtain the image information of the user during exercise, determining the user's current action according to the image information, and then determining the user's non-standard action according to the comparison result between the standard action and the current action, and increasing the proportion of fitness content corresponding to non-standard actions in the fitness course.

In some embodiments, the system for assisted fitness may adjust adaptively the courses selected by the user according to the obtained current state data of the user, so that the courses recommended to the user may be adapted to the current state of the user. The current state of the user may be measured by the user's exercise index parameters, exercise physiological parameters, or the like. For example, if the existing user chooses the fitness content independently, the system for assisted fitness may obtain the exercise physiological parameters of the user and monitor exercise physiological parameters of the user. When it is monitored that the course selected by the user does not match the user's current exercise physiological parameters, the system for assisted fitness may re-predict the courses that the user may need according to the user's current exercise physiological parameters, and recommend the re-predicted courses to the user. The predictions may be made through models or algorithms (all predictions in this disclosure may be made through models).

In some embodiments, the re-predicted courses may be obtained through the course prediction model. The course prediction model may refer to a model for predicting a course that meets the user's current requirements according to the user's current state data.

In some embodiments, the input of the course prediction model may include the user's current state data, for example, the user's current exercise index parameters, exercise physiological parameters, or the like. The output of the course prediction model may include the course that meets the user's current state. For example, the user's current exercise index parameters and exercise physiological parameters may be processed based on the course prediction model to determine the course that conforms to the user's current state.

In some embodiments, the course prediction model may include various feasible neural network models. For example, convolutional neural networks (CNN), deep neural networks (DNN), or the like, or a combination thereof.

] In some embodiments, the course prediction model may be trained separately based on historical data. In some embodiments, the course prediction model may be obtained by training based on a plurality of training samples and labels.

In some embodiments, the training sample may include sample exercise index parameter and sample exercise physiology parameter. The label may be course that meets exercise index parameters and exercise physiology parameters. The training sample may be obtained based on historical data, and the label of the training sample may be determined by manual labeling or automatic labeling. The above description is only an example, not a limitation, and the label of the training data may be obtained in various ways.

For example, if it is determined from the collected images that the user's substandard action has exceeded the preset standard (i.e., the exercise index parameter does not meet the standard), then the system for assisted fitness may infer that the user's current physical state may have difficult to complete the course corresponding to the current physical level, and may recommend a course corresponding to lower physical levels to the user. Similarly, if it is determined from the obtained image of the user that all the actions of the user have met the standard or the number of the actions meeting the standard have exceeded the preset condition (i.e., the exercise index parameters meet the standard), the system for assisted fitness may infer that the current course is relatively easy for the user, and may recommend course with a higher physical level to the user. If the user's breathing is already very short in the obtained voice data fed back by the user, the system for assisted fitness may predict that the user's physical ability may not be able to keep up with the current course, and may recommend the course corresponding to lower physical levels to the user.

In some embodiments, the system for assisted fitness may make adjustment or correction to recommended or generated the fitness guidance course based on user feedback information. More descriptions of obtaining the user feedback information, reference may be found in the user feedback section. For example, in the fitness process, if the user feels that doing a certain action is too difficult or too easy, or a certain action may make other parts of the body uncomfortable, the user may give feedback to the system for assisted fitness. The system for assisted fitness may correct the fitness content according to the user feedback information.

In some embodiments, the system for assisted fitness may utilize the course correction model to adjust or correct the fitness guidance course based on the obtained user feedback information. The course correcting model may refer to a model for adjusting or modifying the fitness guidance course based on the user feedback information.

In some embodiments, the input of the course correcting model may include user feedback information, e.g., process information, or the like. The output of the course correcting model may include a corrected fitness guidance course.

In some embodiments, the course correcting model may include various feasible neural network models. For example, convolutional neural networks (CNN), deep neural networks (DNN), or the like, or a combination thereof.

In some embodiments, the course correcting model may be trained individually based on historical data. In some embodiments, the course correcting model may be obtained by training based on a plurality of training samples and labels.

In some embodiments, the training sample may include process sample information and non-process sample information. The label may be fitness guidance course. The training sample may be obtained based on historical data, and the label of the training sample may be determined by manual labeling or automatic labeling. The above description is only an example, not a limitation, and the label of the training data may be obtained in various other ways.

The mode of correcting the displayed content of the fitness apparatus may include, but is not be limited to, any one of the above methods and a combination thereof, and other methods may also be used to correct the displayed content, which may not be limited here.

The system for assisted fitness may determine the difference from the standard action according to the actions of the user when performing fitness, and remind the user. For example, the user is provided with corrective information to correct the user's fitness action.

In some embodiments, the standard action may include specific requirements related to the action and the range that the body needs to make, for example, the range or position that each limb or joint needs to achieve in a specific fitness action. In some embodiments, the standard action may include requirements for use of the fitness apparatus, for example, which part of the body in contact with the fitness apparatus and the location of the contact on the fitness apparatus, or the like. In some embodiments, the standard action may also include details that are not easily perceptible locally. For example, some actions may have requirements on breathing mode or breathing frequency, and some actions such as dance may also have requirements on facial expressions, etc. In this case, the specific requirements are used as the standard.

In some embodiments, the system for assisted fitness may determine the standard action for the related specific requirements with the action and the range that the body needs to make, or the like.

The system for assisted fitness may determine the standard action in various ways. In some embodiments, the system for assisted fitness may determine the standard action based on the coaching action. For example, the system for assisted fitness may set the coach's human skeleton data to be in the same proportion as the user's human skeleton, and use the coaching action range as the standard action range. For another example, the system for assisted fitness may determine the coaching action with the highest similarity to the user's body shape as the standard action. For example, the system for assisted fitness may collect the body data (such as height, weight, measurements, etc.) of many coaches with different body shape in advance, as well as collect the action range of each action performed by each coach, and establish a standard action library. The action range of the coach that best matches the user's body data may be used as the standard action range.

In some embodiments, the standard action may be determined or adjusted based on the user's information, fitness program, and/or actual actions. For example, the user's information and the image of the user's actual action may be input into the model, and the adjustment method of the standard action may be output. For another example, based on a preset rule, if the user's age is greater than the threshold, the action range may be reduced.

In some embodiments, the system for assisted fitness may determine the standard action based on the user's own action. For example, the system for assisted fitness may determine the limit range of the user to perform a certain action as a standard action. The limit range may refer to the maximum range that the user may achieve when performing a certain action. In some embodiments, the system for assisted fitness may make a guide video, so that the user may input the limit position of the human body according to the guide video. For example, the angle between the legs and the bending degree of the legs, the degree of the arm when the arm is raised vertically, etc. If there is a difference between the user's human body limit position and the standard action, the user's human body limit position may be used as the standard action range.

In some embodiments, the standard action determined by the system for assisted fitness may be dynamically varied. For example, the system for assisted fitness may classify the standard action obtained earlier, and generate the standard action corresponding to different process according to the user's exercise situation and progress. For another example, the system for assisted fitness may combine the user information obtained in real time when the user completes the action, such as the user's expression, the user's breathing rate, etc., to determine the user's state, and select the difficulty level of the corresponding level as the current standard.

In some embodiments, the system for assisted fitness may determine the standard action required when the fitness apparatus is used.

In some embodiments, the system for assisted fitness may determine the standard instruction for use of the fitness apparatus as the standard action. For example, dumbbells may have corresponding requirements on the grip (including ordinary grip, lock grip, empty grip, power-assisted grip, etc.), grip distance, and grip position during use. In some embodiments, the system for assisted fitness may adjust the standard actions determined by the standard instructions according to the user's exercise purpose and/or the difficulty level currently acceptable to the user, etc.

In some embodiments, the system for assisted fitness may determine the standard action based on the use purpose of the fitness apparatus. For example, foam rollers may be sometimes used as fitness apparatus and sometimes used as relaxation apparatus. When the foam rollers are used as the fitness apparatus and the relaxation apparatus, the corresponding standard action may be different, and even when it is used as a relaxation apparatus, the foam rollers may be used in different ways for different parts of the relaxation. At this time, the system for assisted fitness may determine the corresponding standard action according to the specific use purpose of the fitness apparatus.

In some embodiments, the system for assisted fitness may determine the standard action of local details. For example, some actions may have requirements on breathing patterns or breathing frequency, while a certain action such as dancing may also have requirements on facial expressions, etc. In this case, the system for assisted fitness may determine the specific requirement as the standard action.

In some embodiments, the system for assisted fitness may determine corrective information. The corrective information may include a difference between the user action and standard action and/or guidance on what the user should do to comply with the standard action.

In some embodiments, the system for assisted fitness may determine a difference between the standard action and the user's motion, and further determine corrective information based on the difference. The difference may be measured in various ways, for example, the completion degree of the user action, the similarity between the user action and the standard action, the actual difference between the user action and the standard action (the difference between the height of the user's feet and the standard action being 5 m), or the like.

The system for assisted fitness may determine the difference between the standard action and the user's action by processing the obtained data. More descriptions of the obtained data, reference may be found in the related descriptions in the obtaining information section.

In some embodiments, the system for assisted fitness may obtain corrective information by determining the difference between the standard action and the user's action by obtaining bone and joint data (i.e., the user's key points). For example, the system for assisted fitness may extract the bone and joint data when the user repeats each action, and compare the bone and joint data with the bone and joint data corresponding to the standard action to determine the completion degree of the action.

In some embodiments, the system for assisted fitness may determine the difference between the standard action and the user action by obtaining gesture data to obtain corrective information. For example, if a user wears a marking apparatus at each key position, the system for assisted fitness may obtain the user's posture data by identifying the marking apparatus and compare it with the posture data corresponding to the standard action to determine the completion degree of the action. The marking apparatus may be a smart apparatus worn and/or carried by the user. More descriptions of the smart apparatus, reference may be found in the related descriptions in the previous section on obtaining information.

In some embodiments, the system for assisted fitness may determine the difference between the standard action and the user's action by obtaining information about the fitness apparatus and use method and use location of the fitness apparatus by the user, etc., to determine corrective information. For example, for the action that require the help of external fitness apparatus, the system for assisted fitness may identify different types and models of the fitness apparatus through deep learning, and identify the contact method and contact position between the fitness apparatus and the user, and then determine the corrective information for the use of the fitness apparatus according to the standard use of the fitness apparatus.

In some embodiments, the system for assisted fitness may obtain corrective information by obtaining the user's breathing data. More descriptions of obtaining method, reference may be found the content of the obtaining information section. For example, for some yoga actions that require breathing frequency or breathing method, the system for assisted fitness may obtain the user's breathing data through the aforementioned various obtaining methods, such as image recognition (e.g., identifying the ups and downs of the user's chest and abdomen or the opening and closing of the mouth, etc.), and compare it with the standard breathing data to obtain corrective information.

In some embodiments, the system for assisted fitness may obtain the user's facial expression-related data through the user's facial image obtained by the image obtaining apparatus to obtain corrective information. For example, the system for assisted fitness may collect the user's facial expressions when performing some actions that require facial expressions through high-resolution apparatus, and compare them with standard facial images to obtain the corrective information.

In some embodiments, when comparing the related information of the collected user action with the related information corresponding to the standard action, the system for assisted fitness may adopt various comparison methods to achieve the corresponding comparison.

In some embodiments, the system for assisted fitness may employ coordinate comparisons or similarity calculations. For example, the three-dimensional coordinates of the user's human body in the first picture frame of the user space timing information extracted for action display is compared with the three-dimensional coordinates of the coach's human body in the first picture frame in the coach space timing information. For example, the system for assisted fitness may compare the coordinates of the key nodes of the human skeleton of the coach and the user or calculate the similarity through the human skeleton information comparison algorithm, identify a key node whose coordinate difference is greater than a preset threshold as a position with non-standard actions, or identify a position with a similarity lower than the threshold as a position with non-standard actions. The human skeleton information comparison algorithm may include an algorithm that compares or calculates similarity between certain information in two or more skeletons. For example, Euclidean distance, Manhattan distance, collaborative filtering algorithms, etc.

In some embodiments, the system for assisted fitness may employ parameter value comparisons. For example, the system for assisted fitness may only obtain the user's skeleton data or posture data, and determine whether the user action meets the standard action according to the angle and distance (the data may be derived from the standard actions determined in the previous steps) that each body part should have when each action reaches the standard range, and identify the point where the difference between the relevant distance value or angle value and the standard value exceeds the threshold as the position where the action is not standard.

The modes of determining the standard action and determining the corrective information may include, but are not be limited to any one of the above-mentioned methods and combinations thereof, and the determination of the standard action and the corrective information may also be implemented in other modes, which are not limited here.

In some embodiments, the system for assisted fitness (e.g., a fitness apparatus) may output corrective information to prompt the user.

In some embodiments, the system for assisted fitness may output corrective information in the form of a video prompt. For example, the fitness apparatus may indicate the standard position that the user needs to reach in the form of a dotted frame in the user action video. For another example, the fitness apparatus may generate a small video window in the existing video window or in the newly created video window, and display the user's current wrong posture and guidance suggestions on how to convert from the wrong posture to the correct posture. For another example, when the user video and the coach video are overlapping displayed, the fitness apparatus may directly use the action range of the coach image as the action range that the user needs to perform.

In some embodiments, the fitness apparatus may output corrective information in the mode of voice prompts. For example, the action that the user needs to perform is explained by the unified voice output apparatus, such as voice prompting the user to raise the head, keep the action, straighten the arm, lower the hip, and swing the arm slightly.

In some embodiments, the fitness apparatus may output corrective information in a mode of prompting by an intelligent peripheral. For example, the marking apparatus worn on each key part of the user may have the function of vibration and voice prompts. If it is detected that the user's left hand needs to be raised, the marking apparatus worn on the left hand may vibrate and prompt the user to raise the left hand by another 3 cm.

In some embodiments, the fitness apparatus may output corrective information in the mode of textual prompts. For example, the text corresponding to the above-mentioned voice prompt may be displayed on the display apparatus.

In some embodiments, the system for assisted fitness may use a mixture of the above-mentioned prompts, for example, taking the modes of video, voice, peripherals, and text prompts together. In some embodiments, the system for assisted fitness may determine the mode of prompt in combination with the user's situation. For example, users who are running or exercising should avoid using vibration prompt.

In some embodiments, the prompt may be generated from a corrective suggestion template. In some embodiments, the system for assisted fitness may formulate a corrective suggestion template related to the standard action for each standard action, and the corrective suggestion template may be generated according to the action point and error-prone posture of the corresponding standard action. The corrective suggestion template may refer to what needs to be done to achieve standard action and/or error-prone posture, etc. For example, the action point of the plank may include “bending the elbow, the forearm and the forefoot are on the ground, and the ears, shoulders, hips, knees, and ankles are in a straight line”, then the corresponding corrective suggestion template may be, for example, “raising A to B so that A is on the same line as C and D”. The A, C, D, and other parts may be used to fill in different body part names, and B may be used to fill in the corresponding measured lengths, such as the calculated 5 cm, 10 cm, etc.

The mode of outputting the prompt of the corrective information may include, but is not be limited to, any one of the above-mentioned methods and combinations thereof, and the output prompt may also be implemented in other modes, which are not limited here.

More descriptions of the user feedback, reference may be found in the following descriptions.

In some embodiments, any system (not limited to the system for assisted fitness) may capture and recognize user feedback and make different response. In some embodiments, the user feedback may include interactions with any system or apparatus of the system issued by the user, such as likes, comments, and/or feedback, etc., which are not limited herein.

In some embodiments, the user feedback may be the control instruction issued by the user. The control instruction may be an instruction used by the user to control the apparatus to implement system functions. For example, the user feedback such as volume adjustment, likes, and/or positive comments may be considered as control commands. For another example, the control instruction may also include other feedbacks (e.g., selection, cancellation, etc.) sent by the user, which may not be limited herein.

In some embodiments, the user may issue the control instruction to the terminal apparatus in any system (e.g., the fitness apparatus in the system for assisted fitness) in various ways. For example, the user may issue control instruction by clicking buttons, sounds, or actions.

Any system may recognize control commands in various ways. In some embodiments, any system may recognize the control instruction according to the obtained image information, the obtained voice information, or the like. For example, the system for assisted fitness may obtain the control instruction by recognizing the user's gestures. For another example, the system for assisted fitness may extract keywords in the voice information by using the voice recognition technology, and match the control content of the control instruction based on the keywords.

In some embodiments, any system may prompt the user for the control instruction and its corresponding control content. The mode of prompting s may be voice, image, text, and other prompts. In some embodiments, the display apparatus of the terminal apparatus may display the control instruction and its control content (e.g., displaying an animation of the performed action on the terminal device), and the user may execute the control instruction according to the displayed content. For example, the user may perform the gesture corresponding to the selected control content according to the prompt, and the system for assisted fitness may obtain the user's gesture to determine the control content corresponding to the user's gesture.

In some embodiments, any system may prompt the user accordingly based on whether the identified control instruction is standard. For example, the standard action of like is thumb up. When the user's thumb is inclined upward, the system for assisted fitness may remind the user to deflect the thumb accordingly. When the user just matches the standard action, the system for assisted fitness may encourage the user.

In some embodiments, any system may recognize control instructions issued by different users. As mentioned above, the system for assisted fitness may recognize a plurality of users and the instructions issued by the users, and other systems may recognize the control instructions issued by different users in a similar way. More descriptions of identifying control instructions issued by different users, reference may be found in the related content in the user section above.

In some embodiments, any system may determine different control instructions according to the preferences of different users.

In some embodiments, the user may set control instructions corresponding to different gestures according to their preferences. For example, if the user is accustomed to using the thumb up as a “favorite” instruction instead of “like”, the user may set the “favorite” gesture on the terminal apparatus as thumb up.

In some embodiments, any system may modify the standard gestures according to the gestures when the user issues an instruction. For example, the thumb up means like, but the user always makes the thumb oblique, the system for assisted fitness may learn the user's gesture, and use the thumb oblique as a standard gesture to facilitate the user to be quickly recognized by the system for assisted fitness when the users issue an instruction for the next time.

In some embodiments, the user feedback may be a fitness instruction from a fitness coach. The fitness instruction may be an instruction for the user to fitness according to the fitness guidance course. For example, a tip on training action and a training speed in fitness guidance courses. More descriptions of the fitness guidance course, reference may be found in the related descriptions of the fitness guidance course section above. The fitness instruction may be in various ways, such as voice or image data. In some embodiments, the fitness instruction may also be corrective information in the fitness apparatus, e.g., a dotted box or a shadow, or the like. More descriptions of the corrective information, reference may be found in the description below.

In some embodiments, the system for assisted fitness may obtain the user's fitness action, and determine whether the user performs corresponding fitness instruction based on the fitness action. For example, the system for assisted fitness may match the user's current fitness action and the prompt action of the fitness instruction, when the similarity is greater than a certain threshold, it is considered that the user executes the related fitness instruction.

In some embodiments, when the user thinks that the user is unable to complete a certain action in the fitness guidance course, the fitness instruction may be executed through an instruction “skip”. For example, when the system for assisted fitness recognizes that the user performs a finger sliding action, it is considered that the user has executed the fitness instruction, and skips to the next fitness instruction. In some embodiments, the user may perform the action of the instruction “switching” to switch the difficulty of the fitness instruction and execute the switched fitness instruction. For example, the user may issue an instruction to slow down the speed of the high leg raising, so that the system for assisted fitness may reduce the difficulty of the high leg raising.

In some embodiments, the system for assisted fitness may automatically adjust or generate the fitness instruction in various ways. For example, the system for assisted fitness may recognize the exercise action of the fitness coach in the picture or video and automatically generate the fitness instruction, so that the user may execute the fitness instruction to fitness. For example, the system for assisted fitness may extract the action by using human body recognition technology, and recognize the frequency and range of action to generate the fitness instruction. For another example, the system for assisted fitness may issue an instruction for switching the action to the student when recognizing that the coach switches the action. For another example, the system for assisted fitness may generate or adjust the fitness instructions according to the user's exercise index parameters, exercise physiological parameters, or the like. For example, the system for assisted fitness may determine the aerobic exercise frequency that needs to be prompted in the fitness course according to the user's vital capacity. In another example, when the user's exercise physiological parameter indicates that the user's physical ability is insufficient (e.g., a fast heartbeat, etc.), and the exercise index parameter indicates that the user's exercise may not be up to standard, the system for assisted fitness may adjust the fitness instruction to reduce the user's fitness difficulty, etc.

More descriptions of other functions of the system for assisted fitness, reference may be found in the following descriptions.

The system for assisted fitness may implement the safety and security function for the user in various ways. In some embodiments, the system for assisted fitness may provide the user with early warning information according to the user's situation. In some embodiments, the system for assisted fitness may monitor the user's exercise physiological data in real time, and based on the exercise physiological data, predict the possibility of the user's unexpected situation, and provide the user or the user's emergency contact with early warning information. For example, when it is recognized that the user's heart rate exceeds the safe range, the system for assisted fitness may remind the user to stop the current action to reduce the heart rate. In some embodiments, the system for assisted fitness may monitor the user's voice data, accidentally call the police through voice trigger (voiceprint recognition, content (keyword) recognition, etc.), and contact an emergency contact. For example, the system for assisted fitness may call a medical institution after identifying the user's abnormal scream, and contact an emergency contact after calling the medical institution.

In some embodiments, the system for assisted fitness may identify and monitor the unexpected situation of the user, and adjust the fitness content or remind other similar users based on the unexpected situation to avoid similar accidents to the user during the subsequent fitness process or other users performing the exercise. For example, the system for assisted fitness may capture the video or image of the entire process of the user's accident through the camera, and identify and analyze the image (including images from video) through the model or algorithm to determine the type of user's accident and further determine the correlation between the accident and the fitness content. In some embodiments, the safety warning function for the user may also be implemented in other ways, which may not be limited herein. For example, the system for assisted fitness may obtain the type of safety accident that occur frequently during the fitness process through the network and regularly remind the user.

The system for assisted fitness may implement an interactive function with the user in various ways. In some embodiments, the system for assisted fitness may send encouragement information (e.g., like images, encouragement speech, etc.) to the user in one or more ways that are not limited to images, text, and sounds based on the user's fitness situation. In some embodiments, the system for assisted fitness may trigger the incentive mechanism by the degree of similarity of the user and standard action. For example, when the image fit of the range of the user action and the range of the standard action reaches 90%, the system for assisted fitness may voice prompt the user with the encouraging information such as “the action is very standard, please continue to cheer”, etc.

In some embodiments, the system for assisted fitness may remind the user of matters needing attention through the obtained user information. For example, when the system for assisted fitness recognizes that the time of a single exercise of the user exceeds a certain threshold, it may remind the user to pay attention to rest, or remind the user to replenish water at the end of the fitness guidance course.

In some embodiments, the system for assisted fitness may remind the user of the calories consumed in the exercise after exercising or during exercise, and the calories may also be replaced by specific identifiers, such as food equivalent to the amount of calories. The correspondence between different calories and equivalent foods may be preset and stored. The calories consumed in the exercise may be determined based on user behavior information. For example, the action image, the subscribed fitness guidance course, and the exercise index parameter are input into the model, and the model outputs the calorie value or the calorie level.

In some embodiments, the system for assisted fitness may capture the user action images every time through the camera, record the user's fitness changes, remind the user to achieve the goal after reaching a certain goal (e.g., the number of days to keep exercising, etc.), and give rewards, such as unlocking more difficult actions.

In some embodiments, the system for assisted fitness may periodically remind the user to exercise according to the user's exercise situation, for example, the screen of the fitness apparatus flashes or displays encouraging content.

In some embodiments, the system for assisted fitness may compare the user's body shape after exercise and before exercise. For example, when it is recognized that the user's body shape has changed after long-term exercise, the system for assisted fitness may recommend more suitable clothing for the user according to the user's body shape. In some embodiments, the interactive function with the user may also be implemented in other ways, which may not be limited herein.

The system for assisted fitness may implement the function of pause and resumption of the monitoring exercise in various ways. In some embodiments, the system for assisted fitness may also identify the actual situation of the user to determine whether to pause the fitness video. For example, if a user suddenly leaves the image capture area (e.g., the camera's field of view) while exercising, when the user is not captured in the image capture area, the system for assisted fitness may pause the fitness video until it recognizes that the user is present in the image capture area. In some embodiments, in order to prolong the service life, the system for assisted fitness may set a preset pause time, and if the pause time exceeds the preset pause time, the display may be turned off. In some embodiments, the system for assisted fitness may remind the user to continue the last unfinished exercise. For example, if it is recognized that the user is present in the image capture area, a prompt message may be issued when the apparatus is turned on. For example, the system for assisted fitness may record the fitness records of different users and the progress of the fitness guidance courses. When the user is exercising, the system for assisted fitness may identify the user and extract the user's fitness record and fitness progress based on the user identification. In some embodiments, the function of pause and resumption of the monitoring exercise may also be implemented in other ways, which are not limited herein.

The system for assisted fitness may realize the function of environmental control in various ways. In some embodiments, the system for assisted fitness may also be connected in communication (e.g., connection via Internet of Things) with the electrical appliance, and may control the electrical appliance to work according to the exercise content to create an environment suitable for the user to perform a certain fitness exercise. For example, when the course is hot yoga, the system for assisted fitness may turn on heating apparatus (e.g., air conditioning, heating, etc.) to create a high temperature environment. In some embodiments, the function of environmental control may also be implemented in other ways, which are not limited herein. For example, the system for assisted fitness may issue an environmental adjustment reminder (e.g., a reminder to adjust the room temperature to 27° C.) according to the content of the exercise to remind the user to manually implement environmental control.

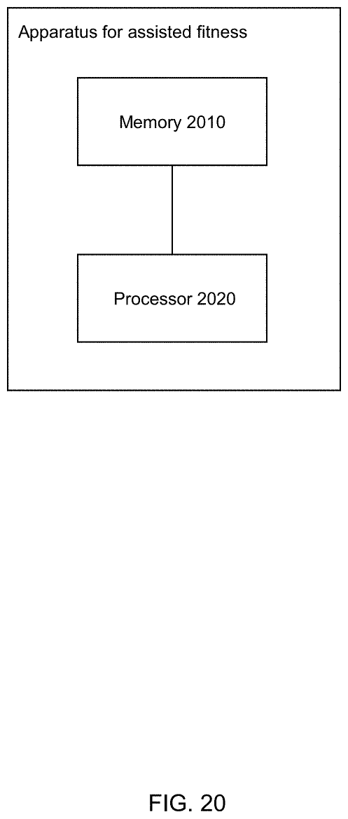

FIG. 20 is an exemplary block diagram of an apparatus for assisted fitness according to some embodiments of the present disclosure. It should be understood that the apparatus may execute the methods and operations in other various embodiments of the present application, which may not be described in detail here in order to avoid repetition. As shown in FIG. 20, the apparatus for assisted fitness may include: a memory 2010 and a processor 2020.

The memory 2010 may be used for storing a computer instruction. In some embodiments, the computer instruction may include a control instruction, an interactive instruction, etc., to control the system for assisted fitness to perform operations corresponding to the instruction. In some embodiments, the memory 2010 may also be used to store related data during the user's exercise, such as interactive content, a fitness guidance course, user information, a physiological parameter and picture and video data during the user's exercise. In some embodiments, the memory 2010 may also store other data related to the user's fitness process, which may not be limited in the present disclosure.

The processor 2020 may be configured to execute the instruction in the memory 2010, and when the instruction is executed by the processor 2020, the apparatus for assisted fitness may be caused to execute the method applied to the apparatus for assisted fitness in any of the embodiments of the present disclosure. For example, when the instruction in the memory 2010 executed by the processor 2020 is an interactive instruction, the assisted fitness apparatus may be made to execute the method of displaying the interactive content corresponding to the interactive instruction to the user. For another example, when the processor 2020 executes the control instruction of the instruction of “viewing fitness records” in the memory 2010, the apparatus for assisted fitness may be made to execute the method of displaying the fitness records for the user. In some embodiments, the processor may also execute other instructions in the memory 2010 to cause the apparatus for assisted fitness to execute various methods applied to the apparatus for assisted fitness shown in any other embodiments of the embodiments of the present disclosure, which is not limited in this specification.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or feature described in connection with the embodiment is included in at least one embodiment of the present disclosure. Therefore, it is emphasized and should be appreciated that two or more references to “an embodiment,” “one embodiment,” or “an alternative embodiment” in various portions of the present disclosure are not necessarily all referring to the same embodiment. Furthermore, the particular features, structures, or features may be combined as suitable in one or more embodiments of the present disclosure.

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution—e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the count of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the descriptions, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A method for assisted fitness, executed based on a fitness apparatus, comprising:
identifying at least one user using the fitness apparatus based on collected data, and determining an identification result;
obtaining a control instruction; and
in response to the control instruction, executing control content of the fitness apparatus corresponding to the control instruction based on the identification result.

2. The method of claim 1, wherein the collected data includes at least one of image data of the at least one user, biometric data of the at least one user, terminal apparatus data carried by the at least one user, and voice data of the at least one user.

3. The method of claim 1, wherein at least one pointer and at least one function icon are displayed on a display apparatus of the fitness apparatus, and the at least one pointer has a corresponding relationship with the at least one user;
the control instruction includes an instruction for controlling the at least one pointer;
the in response to the control instruction, executing control content of the fitness apparatus corresponding to the control instruction based on the identification result includes:
determining a target user based on the identification result, wherein the target user is a user who issues the control instruction among the at least one user; and
determining the control content based on the function icon pointed to by a target pointer, wherein the target pointer is a pointer corresponding to the target user.

4. The method of claim 1, wherein the in response to the control instruction, executing control content of the fitness apparatus corresponding to the control instruction based on the identification result includes:
determining a target user based on the control instruction, wherein the target user is a user who issues the control instruction among the at least one user;
determining the control content corresponding to the target user based on the target user and the control instruction; and
executing the control content.

5. The method of claim 1, wherein the control content includes display content on a display apparatus of the fitness apparatus; the display content includes at least one of a fitness guidance course and a fitness action image of the at least one user;
the in response to the control instruction, executing control content of the fitness apparatus corresponding to the control instruction based on the identification result includes:
determining a target user based on the control instruction, wherein the target user is a user who issues the control instruction among the at least one user;
determining the display content based on the control instruction;
determining a display area of the target user in the display apparatus of the fitness apparatus based on the target user; and
displaying the display content on the display apparatus based on the display area.

6. The method of claim 1, further comprising:
in response to the control instruction including an interactive instruction and at least two users corresponding to the interactive instruction using a same fitness apparatus, outputting interactive content corresponding to the interactive instruction, wherein the interactive content includes interactive fitness data.

7. The method of claim 1, further comprising:
in response to the control instruction including an interactive instruction, obtaining interactive reference information, wherein the interactive reference information includes at least one of an interactive mode between interactive users, a number of the interactive users, and the identification result of the interactive users; the interactive users are at least two users participating in interaction;
determining interactive content corresponding to the interactive instruction based on the interactive reference information; and
outputting the interactive content.

8. The method of claim 7, wherein the interactive mode includes at least one of an interactive mode based on an interactive action and an interactive mode based on an interactive request.

9. The method of claim 1, further comprising:
obtaining an interactive action of a user to be evaluated, wherein the user to be evaluated is at least one user participating in interaction; and
evaluating an action standard degree of the interactive action, and obtaining evaluation information of the user to be evaluated.

10. The method of claim 9, further comprising:
determining fitness guidance information of the user to be evaluated based on the evaluation information.

11. A fitness apparatus, comprising: a memory for storing instructions; at least one processor, the at least one processor in communication with the memory for executing the instructions; when executed by at least one processor, causing the fitness apparatus to perform operations including:
identifying at least one user using the fitness apparatus based on collected data, and determining an identification result;
obtaining a control instruction; and
in response to the control instruction, executing control content of the fitness apparatus corresponding to the control instruction based on the identification result.

12. The apparatus of claim 11, wherein the collected data includes at least one of image data of the at least one user, biometric data of the at least one user, terminal apparatus data carried by the at least one user, and voice data of the at least one user.

13. The apparatus of claim 11, wherein at least one pointer and at least one function icon are displayed on a display apparatus of the fitness apparatus, and the at least one pointer has a corresponding relationship with the at least one user;
the control instruction includes an instruction for controlling the at least one pointer;
the operations further include:
determining a target user based on the identification result, wherein the target user is a user who issues the control instruction among the at least one user; and
determining the control content based on the function icon pointed to by the target pointer, wherein the target pointer is a pointer corresponding to the target user.

14. The apparatus of claim 11, wherein the operations further include:
determining a target user based on the control instruction, wherein the target user is a user who issues the control instruction among the at least one user;
determining the control content corresponding to the target user based on the target user and the control instruction; and
executing the control content.

15. The apparatus of claim 11, wherein the control content includes display content on a display apparatus of the fitness apparatus; the display content includes at least one of a fitness guidance course and a fitness action image of the at least one user;
the operations further include:
determining a target user based on the control instruction, wherein the target user is a user who issues the control instruction among the at least one user;
determining the display content based on the control instruction;
determining a display area of the target user in the display apparatus of the fitness apparatus based on the target user; and
displaying the display content on the display apparatus based on the display area.

16. The apparatus of claim 11, wherein the operations further include:
in response to the control instruction including an interactive instruction and at least two users corresponding to the interactive instruction using a same fitness apparatus, outputting interactive content corresponding to the interactive instruction, wherein the interactive content includes interactive fitness data.

17. The apparatus of claim 11, wherein the operations further include:
in response to the control instruction including an interactive instruction, obtaining interactive reference information, wherein the interactive reference information includes at least one of an interactive mode between interactive users, a number of the interactive users, and the identification result of the interactive users; the interactive users are at least two users participating in interaction;
determining the interactive content corresponding to the interactive instruction based on the interactive reference information; and
outputting the interactive content.

18. The apparatus of claim 17, wherein the interactive mode includes at least one of an interactive mode based on an interactive action and an interactive mode based on an interactive request.

19. The apparatus of claim 11, wherein the operations further include:
obtaining an interactive action of a user to be evaluated, wherein the user to be evaluated is at least one user participating in interaction; and
evaluating an action standard degree of the interactive action, and obtaining evaluation information of the user to be evaluated.

20. A non-transitory computer-readable storage medium storing a set of instructions, when executed by at least one processor, causing the at least one processor to perform the method of claim 1.
